SEQUENCE LISTING (8 sequences)

SEQ ID NO: 1 - DNA, 15 bp, synthetic construct
  misc_feature 1..15: note = exemplary T-oligonucleotide
  source 1..15: mol_type = other DNA
ttccacattacttct

SEQ ID NO: 2 - DNA, 15 bp, synthetic construct
  misc_feature 1..15: note = exemplary T-oligonucleotide
  source 1..15: mol_type = other DNA
atccccattacttct

SEQ ID NO: 3 - DNA, 20 bp, synthetic construct
  misc_feature 1..20: note = exemplary T-oligonucleotide
  source 1..20: mol_type = other DNA
gggtggcgtatgatagctat

SEQ ID NO: 4 - DNA, 20 bp, synthetic construct
  misc_feature 1..20: note = exemplary T-oligonucleotide
  source 1..20: mol_type = other DNA
aggttaagtatgatagttat

SEQ ID NO: 5 - DNA, 20 bp, synthetic construct
  misc_feature 1..20: note = exemplary T-oligonucleotide
  source 1..20: mol_type = other DNA
gggttaagcatggtagttat

SEQ ID NO: 6 - DNA, 20 bp, synthetic construct
  misc_feature 1..20: note = exemplary T-oligonucleotide
  source 1..20: mol_type = other DNA
aggttaagcgttatatttat

SEQ ID NO: 7 - DNA, 15 bp, synthetic construct
  misc_feature 1..15: note = exemplary FRET-oligonucleotide
  source 1..15: mol_type = other DNA
agaagtaatgtggaa

SEQ ID NO: 8 - DNA, 20 bp, synthetic construct
  misc_feature 1..20: note = exemplary FRET-oligonucleotide
  source 1..20: mol_type = other DNA
ataactatcatacttaacct

(19) United States
(12) Patent Application Publication
(10) Pub. No.: US 2024/0102078 A1
(43) Pub. Date: Mar. 28, 2024

(54) METHOD AND KITS FOR MULTIPLEXED FLUORESCENT MICROSCOPY

(71) Applicant: MICROGRAPHIA BIO LIMITED, London (GB)

(72) Inventors: Sophie ADJALLEY, London (GB); Christopher C. THOMPSON, London (GB)

(21) Appl. No.: 18/452,070

(22) Filed: Aug. 18, 2023

Related U.S. Application Data
(63) Continuation of application No. PCT/EP2022/054140, filed on Feb. 18, 2022.

(30) Foreign Application Priority Data
Feb. 19, 2021 (GB) 2102391.6

(51) Int. Cl. C12Q 1/6804; C12Q 1/6818

(52) U.S. Cl. CPC C12Q 1/6804; C12Q 1/6818

(57) ABSTRACT

Provided is a method for multiplexed fluorescence microscopy comprising contacting a fixed sample with a set of binding agent-T-oligonucleotide conjugates to allow the binding agents to bind to any binding partners present in the sample, wherein the set comprises a plurality of binding agents having different specificities and the sequence of the T-oligonucleotide is unique to the binding agent to which it is conjugated, contacting the sample and any bound binding agents resulting from step a with a FRET-oligonucleotide, illuminating the sample with a wavelength to cause excitation of the FRET-oligonucleotide's emitter molecule, and observing the fluorescent kinetic profile of the sample at the FRET-oligonucleotide emitter molecule's emission wavelength at one or more pixels over time, wherein the FRET-oligonucleotide can hybridise to multiple T-oligonucleotides in the set, to form multiple pairs, and wherein the dissociation and reassociation between each different pair generates a fluorescent kinetic profile that is unique within that set to that pair. Also provided are associated kits, sets of binding agent T-oligonucleotide conjugates and corresponding FRET-oligonucleotides, and methods of designing such sets.

This application is a bypass continuation of International Application No. PCT/EP2022/054140, filed Feb. 18, 2022, which claims priority to, and the benefit of, GB Application No. 2102391.6, filed Feb. 19, 2021, the entireties of each are incorporated herein by reference as if written in their entireties.

### TECHNICAL FIELD

This invention is in the field of multiplexed fluorescent microscopy.

### BACKGROUND ART

### Fluorescence Microscopy

Fluorescence microscopy is a staple component of life science and pharmaceutical research. Many areas of life science and pharmaceutical research rely heavily on fluorescence microscopy to observe biological samples at the sub-cellular level, allowing sub-cellular components to be imaged at high resolution. Orthogonal labelling strategies also enable different components to be imaged in the same cell, using multiple channels (e.g. based on the use of multiple fluorophores) and the composition of these multi-channel micrographs are extremely useful in a variety of downstream analyses.

The ability to image different channels from the same sample typically requires exploitation of known excitation/emission spectra from various fluorescent proteins or dyes. The number of channels that can be imaged in a standard fluorescent microscopy set-up is limited by the amount of overlap in emission/excitation distributions of available fluorophores. In fact, fluorescence is notoriously hard to multiplex; that is, to measure multiple analytes simultaneously. Theoretically, any number of different fluorophores can be used in a fluorescent microscopy experiment, however if the different fluorophores excite or emit with overlapping wavelengths of light, there is no way to deconvolute or separate the fluorescent signals into their respective channels. Standard fluorescent microscopy is therefore limited in practical terms to the use of four to five different fluorophores, each with significant distinction in excitation/emission wavelengths to allow four to five image modalities to be assessed in a single sample. In practice therefore this problem of spectral overlap limits the number of distinct components that can be visualised simultaneously.

### Recent Advances in Single-Molecule Localization Microscopy

Oligonucleotide-based fluorescent labelling strategies have been developed which improve the resolution of fluorescent microscopy since they are capable of breaking the Abbe diffraction limit. In brief, techniques such as DNA-PAINT (Point Accumulation Imaging in Nanoscale Topography) (see e.g. Schnitzbauer, J., Strauss, M. T., Schlichthaerle, T., Schueder, F., and Jungmann, R. (2017), Super-resolution microscopy with DNA-PAINT. Nat. Protocols 12, 1198-1228), an example of a Single-Molecule Localization Microscopy (SMLM) technique, use fluorescently tagged DNA probes that reversibly hybridise to complementary docking strands that are tethered to binding molecules (e.g. antibodies). When the binding molecules are stationary (e.g. because they are attached to their binding partner in a fixed cell sample), the reversible hybridisation between the labelled strand and the docking strand produces transient emission events. The transient emission events can be detected. Localisation data that is generated can be used to create an image of the sample at high resolution. The transient nature of the emission events allows the temporal separation of molecules that could not otherwise have been resolved spatially, e.g. because they are located so close to each other that the signals would be indistinguishable were they to have been imaged using standard techniques.

The ability to record movies capturing multiple stochastic binding events (e.g. binding events at different sites in the sample) means that these events can ultimately be reconstructed as a single super resolution image. Each emission profile can be fitted to a point spread function that allows precise localisation of single-molecules at resolutions below the diffraction limit.

A significant limitation of the DNA-PAINT technique is the high background associated with this technique. This results from the use of the fluorescently labelled imager probes. Fluorescence from these unbound probes contributes large amounts of background fluorescence. This high background not only gives rise to difficulties in detecting signal peaks of individual bound probe molecule but also gives rise to other practical limitations. Attempts to solve this problem of high background are themselves often a trade-off; reducing the concentration of imager probes can reduce background but this in turn limits the rate at which image acquisition can occur and thus prevents rapid image acquisition since the reduction in concentration of the imager probe lowers the rate at which complexes form (and hence the frequency of binding events). The image capture process with DNA-PAINT is therefore often slow. Furthermore SMLM is typically limited to a maximum of three laser wavelengths for excitation, and thus it is not typically possible to image more than three modalities in a single experiment.

The inventors have devised a method for multiplexed fluorescence microscopy which can use a single wavelength excitation illumination protocol. The method exploits the stochastic emission of fluorescence provided by DNA-based fluorescent strategies, and the use of Forster Resonance Energy Transfer (FRET) oligonucleotides allows enhanced spectral-independent and multiplexed modality imaging, e.g. when compared to imaging using oligonucleotide probes which are fluorescently labelled but which can emit signal at all times. As discussed above, the use of standard fluorescent oligonucleotides in techniques like DNA-PAINT is associated with high background, given the continuous fluorescence of the oligonucleotides that are used. This high background is avoided in the methods of the present invention by using FRET oligonucleotides. These FRET-oligonucleotides as used in the invention may have little or no background fluorescence when they are free in solution, but fluoresce when they are hybridised with an oligonucleotide to form a binding pair. The avoidance of high background means that higher concentrations of fluorescent oligonucleotides can be used in the methods of the invention which in turn means that the step of observation (including image or video capture) is more rapid.

The principle of FRET and the use of FRET in connection with oligonucleotides are well known in the art. In such oligonucleotides use is made of the principle of FRET whereby an emitter molecule and a quenching entity are used. On illumination with the emitter's excitation wavelength, no fluorescence at the emitter's emission wavelength is observed when the emitter molecule and quenching entity are located sufficiently close to one another (typically in the range of 1-10 nm). This is due to efficient energy transfer from the emitter molecule to the quenching entity. However, if the emitter molecule and quenching entity are sufficiently physically distant, the rate of FRET is reduced, which in turn allows the emission from the emitter molecule to be detected at the emitter's emission wavelength.

This principle is manipulated in the present invention to generate fluorescent kinetic profiles. A FRET-oligonucleotide contains an emitter molecule and a quenching entity. When the FRET-oligonucleotide is in solution, upon illumination with the emitter's excitation wavelength, no fluorescence is observed at the emitter's emission wavelength due to efficient energy transfer to the quenching entity located proximally (e.g. due to a free/flexible conformation that allow the emitter molecule and a quenching entity, which are typically located at or near the two ends of the oligonucleotide to be in proximity). The FRET-oligonucleotide has the ability to hybridise reversibly with members of a set of Transactivating oligonucleotides (T-oligonucleotides) which are conjugated to binding agents. On hybridisation with a T-oligonucleotide, the structure of the FRET-oligonucleotide becomes constrained by this hybridisation. This causes its emitter molecule and quenching entity to become separated physically and the rate of FRET is reduced, allowing the emission from the emitter molecule at the emitter's emission wavelength to be detected at the site of hybridisation, on illumination at the emitter's excitation wavelength. The T-oligonucleotides and FRET-oligonucleotides are designed to enable reversible hybridisation between the pairs that are formed. This means that for each pair a fluorescent kinetic profile is observed over time; fluorescent emission occurs when the binding pair are in their hybridised configuration but not when the pair are separated. As with DNA PAINT, discussed above, the fact that a fluorescent kinetic profile is detected means that high resolution is achieved Unlike DNA PAINT, the background fluorescence is low and the sensitivity or quality of the signal may therefore be better than in DNA PAINT, e.g. because the ratio of signal to noise is higher.

Furthermore, the method and kits of invention allow for multiplexing. Each FRET-oligonucleotide can hybridise to multiple T-oligonucleotides in the corresponding set, thus forming multiple pairs. The dissociation and reassociation between each different pair generates a fluorescent kinetic profile that is unique within that set to that pair. This means that multiple fluorescent kinetic profiles generated in a single set can be observed simultaneously in a single channel (i.e. based on the same emission spectrum) and the identity of the T-oligonucleotide in each pair can be determined by its fluorescent kinetic profile (e.g. by calculating one or more metric of the fluorescent kinetic profile which is uniquely attributable to the pair) since that fluorescent kinetic profile is unique within that set to that pair. The observation (e.g. detection or capture) of any fluorescent kinetic profiles in the sample may be by recording movies capturing fluorescent kinetic profiles at one or more, e.g. multiple locations or pixels within the sample. This information is used to calculate one or more metric of the fluorescent kinetic profile, which in turn is used to assign an identity to one or more pixels in the observed sample based on the metric calculated from the fluorescent kinetic profile of the sample at that pixel and ultimately reconstruct one or more images of the sample or a part thereof. A metric of the fluorescent kinetic profile is simply a measure or expression of one or more aspects of the fluorescent kinetic profile and can be calculated from or derived from the fluorescent kinetic profile, e.g. by mathematical analysis of one or more aspects of the fluorescent kinetic profile.

The T-oligonucleotides are conjugated to binding agents, such that the sequence of the T-oligonucleotide is unique to the binding agent to which it is conjugated. Therefore it can be determined which binding agent is present at any given location within a sample based on the fluorescent kinetic profile or metric thereof that is observed at that location. Non-limiting examples of these metrics include (i) the average period of time between each fluorescence emission (also referred to as the off-time), (ii) the average duration of the fluorescence emission (also referred to as the on-time), and (iii) the rate of occurrences of fluorescence emission. At least one, at least 2 or at least 3 such metrics can be extracted from a fluorescent kinetic profile and used to describe it. Metrics other than these exemplary metrics may also be used.

The high resolution and low background that is achieved with this technique, together with the in-built multiplexing means that the technique is particularly advantageous.

Using a single FRET-oligonucleotide and a set of T-oligonucleotides can generate a plurality of different fluorescent kinetic profiles (e.g. up to 10 or up to 25). However, multiplexing is made possible at a number of levels. At a first level, the invention uses a FRET oligonucleotide that can bind to multiple T-oligonucleotides in a set, so that a different fluorescent kinetic profile is generated by each pair that is formed within that set. Thus by observing any fluorescent kinetic profiles emitted from the sample over time (or metric thereof), the identity of the T-oligonucleotide that gives rise to any observed fluorescent kinetic profile, and hence the specific binding partner to which the T-oligonucleotide is conjugated can be determined for any location in the sample.

Further multiplexing can be achieved by using more than one FRET oligonucleotide, with each FRET-oligonucleotide having a corresponding set of T oligonucleotides. By using two or more FRET oligonucleotides, with each FRET-oligonucleotide having a corresponding set of T oligonucleotides, a greater range of different fluorescent kinetic profiles can be generated, and thus a greater number of binding agents can be used and a greater number of different target molecules can be detected. This method therefore is particularly advantageous for multiplexing.

By way of example, a first set of T-oligonucleotides and the corresponding FRET-oligonucleotide may give rise to a number of different fluorescent kinetic profiles. Any number of numerical metrics or features may be extracted from fluorescent kinetic profiles. Non-limiting examples of these metrics include (i) the average period of time between each fluorescence emission (also referred to as the off-time), (ii) the average duration of the fluorescence emission (also referred to as the on-time), and (iii) the rate of occurrences of fluorescence emission. At least one, at least 2 or at least 3 metrics can be extracted from a fluorescent kinetic profile and used to describe it, and those metrics can be the metrics recited above or alternative metrics.

Moreover, the turning off and on of a fluorescence emission, conceptualised as “blinking”, can be used to define numerical metrics that describe the hybridisation affinity between each T-oligonucleotide and the corresponding FRET-oligonucleotide. Any number of affinity metrics can be derived from blinking kinetics, including at least (i) the Kₒₙ (Association rate constant, expressed in M-1.s-1) and (ii) the K_{off} (Dissociation rate constant, expressed in s-1). As each T-oligonucleotide has been engineered to exhibit unique hybridisation, and ergo blinking, kinetic profiles, any individual or combination of metrics may be used to distinguish the identity of a T-oligonucleotide under observation. These affinity metrics can also be a way of describing the fluorescent kinetic profile.

A given set of T-oligonucleotides may, for example comprise 10 T-oligonucleotides (T-oligonucleotide 1 to 10). FRET-oligonucleotide A may for example reversibly hybridise to these 10 T-oligonucleotides and generate 10 different fluorescent kinetic profiles (profiles 1 to 10). By adding a further set of T-oligonucleotides, e.g. comprising 10 T-oligonucleotides (T-oligonucleotides 11 to 20) and the corresponding FRET-oligonucleotide (FRET-oligonucleotide B), a further 10 different fluorescent kinetic profiles (profiles 11 to 20) can be used.

A further degree of multiplexing is provided for by using FRET-oligonucleotides having different fluorophores that give rise to fluorescence emission that is detectable in different spectral channels (e.g. using different fluorophores). Collecting signals in multiple spectral channels allows a further level of multiplexing and the ability to detect even more targets of the binding agents in a single sample.

For example, a given set of T-oligonucleotides may, for example, comprise 10 T-oligonucleotides (T-oligonucleotide 1 to 10). FRET-oligonucleotide A may for example reversibly hybridise to these 10 T-oligonucleotides and generate 10 different fluorescent kinetic profiles (profiles 1 to 10). By adding a further set of T-oligonucleotides, e.g. comprising 10 T-oligonucleotides (T-oligonucleotides 21 to 30) and the corresponding FRET-oligonucleotide (FRET-oligonucleotide C), where FRET-oligonucleotide C gives rise to a fluorescent signal that is detectable in a different spectral channel to FRET-oligonucleotide A (e.g. because FRET oligonucleotide C contains a different fluorophore to FRET oligonucleotide A) a further 10 fluorescent kinetic profiles (profiles 21 to 30) can be used. Because profiles 21 to 30 are detectable in a different spectral channel than profiles 1 to 10, it would be possible for one or more of the profiles to be similar or identical to one or more of profiles 1 to 10, because they would be distinguishable on the basis that they are detected in different spectral channels (e.g. because FRET oligonucleotide C contains a different fluorophore to FRET oligonucleotide A). The T-oligonucleotide remains unique to the binding partner and each T-oligonucleotide must give rise to a fluorescent kinetic profile that is unique within its detection channel.

In this approach, the ability to multiplex is therefore not limited by the number of different spectrally-resolvable fluorophores, unlike many other approaches. Multiplexing in the present method relies on the ability to generate distinguishable fluorescent kinetic profiles, which can be achieved by using differences in the base sequences of the T-oligonucleotides. Large numbers of different T-oligonucleotides and FRET-oligonucleotide sequences can be designed and generated, meaning that the invention has built in multiplexing potential, even if only a single fluorophore is used (or if the FRET-oligonucleotides contain emitter molecules that have an emission spectrum that is overlapping), but further levels of multiplexing can be achieved with fluorophores that give rise to emissions that can be detected in different channels (e.g. using multiple fluorophores). The methods of the invention can furthermore be readily integrated with standard microscopy setups e.g. epifluorescent, confocal, total internal reflection fluorescence microscope (TIRF), Stimulated emission depletion (STED) microscope, structured illumination microscope (SIM), and other types of fluorescence microscopes in which a laser, LED, halogen or other illumination source is capable of targeted excitation of fluorophore molecules contained within the sample.

Other techniques have attempted multiplexing of DNA PAINT type probes, however in general these achieved multiplexing using sequential binding of probes, such as those described in WO2015/138653. The adaptability of the current technique to multiplexing, together with the low background fluorescence and improved imaging time are among the advantages of the present method. These advantages make the method of the present invention of particular application to high throughput assays. This utility in high throughput assays is particularly associated with the ability to obtain a high degree of multiplexing, which is desirable as it affords the experiment greater information content per experimental set up.

The methods provided herein therefore take advantage of the fact that the base sequence of an oligonucleotide is a way to identify the oligonucleotide uniquely. That base sequence also determines the nature of an oligonucleotide's hybridisation with other oligonucleotides. The base sequence of T-oligonucleotides and corresponding FRET-oligonucleotides can be programmed in order to generate pairs that have specific fluorescent kinetic profiles, such that the identification of the fluorescent kinetic profile allows identification of the specific T-oligonucleotide that is present in that pair, which in turn allows identification of the specific binding partner to which the T-oligonucleotide is conjugated.

### DISCLOSURE OF THE INVENTION

The invention is based on the inventors' discovery that the principles of FRET can be exploited in the context of methods of multiplexed fluorescent microscopy, in which oligonucleotides are used to uniquely identify a binding partner to which they are conjugated, and in which these oligonucleotides form pairs with a FRET oligonucleotide. Using FRET oligonucleotides, as discussed in more detail, has several advantages. Firstly, when FRET-oligonucleotides are in solution (FIG. 1A) no fluorescence is observed at the emitter's emission wavelength upon illumination with the emitter molecule's excitation wavelength. This is because there is efficient energy transfer from the emitter to the quenching entity allowed by the FRET-oligonucleotide adopting a free conformation in solution. This means that the background levels of fluorescence (e.g. at the emitter's emission wavelength) are low. In contrast, the FRET-oligonucleotides emit fluorescence at the emitter's emission wavelength when they are hybridised with complementary oligonucleotides that have been immobilised throughout the cell sample (FIG. 1B). The hybridisation of the FRET-oligonucleotide with the T-oligonucleotide linearises the FRET oligonucleotide, thus physically separating the quenching component from the emitting component, and reducing the rate of FRET. A fluorescent signal at the emitter's emission wavelength is thus emitted at the site of hybridisation and only when the FRET-oligonucleotide is hybridised. The sequence of the T-oligonucleotides and the FRET-oligonucleotide are designed so that the FRET-oligonucleotide can hybridise to the various T-oligonucleotides in the set. Each different pair that is formed gives rise to a unique fluorescent kinetic profile. This means that by observing the fluorescent kinetic profile that is generated and calculating a distinguishable metric of the fluorescent kinetic profile it is possible to deconvolute the pair's identity at a specific location in a sample (FIG. 4A and FIG. 4B, and FIG. 5A-FIG. 5D) and hence to determine the binding partner that has bound at that specific location in a sample.

Accordingly, the invention provides a method for multiplexed fluorescence microscopy comprising:
a. contacting a fixed sample with a set of binding agent-T-oligonucleotide conjugates to allow the binding agents to bind to any binding partners present in the sample, wherein the set comprises a plurality of binding agents having different specificities and the sequence of the T-oligonucleotide is unique to the binding agent to which it is conjugated;
b. contacting the sample and any bound binding agents resulting from step a with a FRET-oligonucleotide;
c. illuminating the sample with a wavelength to cause excitation of the FRET oligonucleotide's emitter molecule; and
d. observing the fluorescent kinetic profile at the FRET-oligonucleotide emitter molecule's emission wavelength at one or more pixels over time;
   wherein the FRET-oligonucleotide can hybridise to multiple T-oligonucleotides in the set, to form multiple pairs, and wherein the dissociation and reassociation between each different pair generates a fluorescent kinetic profile that is unique within that set to that pair.

The invention also provides a kit for multiplexed fluorescence microscopy comprising:
a. a set of binding agent-T-oligonucleotide conjugates, wherein the set comprises a plurality of binding agents having different specificities and the sequence of the T-oligonucleotide is unique to the binding agent to which it is conjugated; and
b. a FRET-oligonucleotide wherein the FRET-oligonucleotide can hybridise to multiple T-oligonucleotides in the set to form multiple pairs, and wherein the dissociation and reassociation between each different pair generates a fluorescent kinetic profile that is unique within that set to that pair.

The invention also provides a set of binding agent-T-oligonucleotide conjugates and a corresponding FRET-oligonucleotide for making a kit for multiplexed fluorescence microscopy comprising:
a. a set of T-oligonucleotides; and
b. a FRET-oligonucleotide
   wherein the FRET-oligonucleotide can hybridise to multiple T-oligonucleotide in the set to form multiple pairs, and wherein the dissociation and reassociation between each different FRET-oligonucleotide and T-oligonucleotide generates a fluorescent kinetic profile that is unique within that set to that pair.

The invention also provides a method of preparing the kit of the invention, said method comprising providing a set of T-oligonucleotides and a set of binding agents and a corresponding FRET oligonucleotide, and conjugating the set of T-oligonucleotides to the set of binding agents to form a set of binding agent-T-oligonucleotide conjugates, wherein the set of binding agent-T-oligonucleotide conjugates comprises a plurality of binding agents having different specificities and the sequence of the T-oligonucleotide is unique within that set to the binding agent to which it is conjugated.

The invention further provides a method of designing a set of T-oligonucleotides and a FRET-oligonucleotide for use in the method of the invention, said method comprising:
a. Selecting a FRET-oligonucleotide sequence of at least 12 nucleotides in length;
b. Obtaining one or more sequences of at least 8 nucleotides in length that are complementary to the FRET-oligonucleotide sequence;
c. Generating a plurality of potential T-oligonucleotide sequences each of which differs from the sequence that is complementary to the FRET-oligonucleotide by at least one nucleotide; and
d. Selecting at least two T-oligonucleotide sequences on the basis of the ability of the at least two T-oligonucleotide sequences to generate fluorescent kinetic profiles that are different to each other.

### Multiplexed Fluorescent Microscopy

### Fluorescence Microscopy

Fluorescence microscopy is based on the detection of fluorescent compounds, which in turn may be used in order to generate an image of a sample (which may be instead of, or in addition to the detection of light). Fluorophores are fluorescent chemical compounds or biological proteins that can re-emit light upon light excitation. In fluorescence microscopy fluorophores are illuminated with light of one or more specific wavelengths. This light is absorbed by the fluorophore(s), and the energy of the fluorophore is briefly raised to a higher excited state. The subsequent return to ground state results in emission of fluorescent light that can be detected and measured. The detection of the emitted light may be used to generate an image.

Fluorophores are thus molecules that emit light in response to light excitation, the re-emitted light being at a longer wavelength than the light used for excitation. Any given fluorophore will have a defined maximum excitation and emission wavelength which corresponds to the peak in the excitation and emission spectra. Examples include fluorescein (FITC), rhodamine derivatives (TRITC), coumarin derivatives and cyanine derivatives.

Fluorescent microscopy may, for example be used to detect the presence or location within a sample of one or more target molecules. Certain target molecules, such as nucleic acids, can be detected using fluorescent stains (which may be small molecules which are intrinsically fluorescent). Nucleic acid stains such as DAPI and Hoechst are suitable examples, which bind the minor groove of DNA, and are therefore routinely used to fluorescently label cell nuclei. To detect the presence or location within a sample of other target molecules, the ability of specific binding agents to bind to target molecules can be manipulated. It is well known to use antibodies as binding agents, for example Antibodies against a particular target molecule can be used in such methods. The primary antibody (which binds to the target molecule) may be labelled with a fluorescent molecule, or this fluorescent molecule may be carried on a second antibody that binds to the primary antibody. In this way, the detection of fluorescence at a particular location within the sample indicates that the target molecule is present at that location.

Fluorescence microscopy is used in the present methods as the methods involve the observation of a fluorescent kinetic signal that is generated as a consequence of dissociation and reassociation between a FRET-oligonucleotide and T-oligonucleotide as described in more detail below.

### Multiplexing

Multiplexing as used herein means that more than one binding agent is used, which in turn in general means that more than one target molecule may be detected in the same sample, if present (although multiple binding agents may in principle bind to a single target molecule, e.g. where multiple antibodies bind to a large polypeptide, with each antibody binding to a different epitope). Multiplexing may therefore allow for the observation of multiple target molecules in parallel.

These binding agents are in general contacted with the sample e.g. at the same time and may bind to their respective targets, if present, in the same sample. An example of a standard multiplexed assay would be using a first primary antibody against one target molecule and a second primary antibody against a second target molecule. The sample and any bound primary antibody would then be contacted with the corresponding secondary antibodies which were labelled with fluorophores, a first fluorophore for the first secondary antibody and a second fluorophore for the second secondary antibody. By detecting any signal from each of the two fluorophores an image can be generated of the location within the sample of the two target molecules.

A multiplexing process therefore requires the ability to detect more than one fluorescent signal from the same sample. In standard fluorescent microscopy techniques this may be carried out by using more than one fluorophore, for example fluorophores that emit light of sufficiently different wavelengths to allow them to be differentiated. Suitable examples would be methods in which a fluorescein based molecule as a first fluorophore and a rhodamine based molecule is used as a second fluorophore. As mentioned above however, it is difficult to multiplex in the context of fluorophores; the wide emission spectra of most fluorophores limits multiplexing to four or five labels in standard fluorescence microscopes. Furthermore in techniques that rely on the use of secondary antibodies one also needs to make sure that there is no cross-hybridisation between the secondary antibodies by using antibodies derived from different species. This limits the number of possible combinations, and this limitation is overcome in the present invention.

In the context of the present invention, multiplexing is achieved at least by the fact that not only is the presence or absence of a fluorescent signal at the relevant emitter molecule's emission wavelength observed, but also its temporal pattern. Rather than simply observing the presence or absence of fluorescence at the relevant emitter molecule's emission wavelength, multiple “fluorescent kinetic profiles” are observed. The use of fluorescent kinetic profiles means that more information can be obtained from each channel that is used. A plurality of binding agents having different specificities, with each binding agent being conjugated to a T-oligonucleotide is used. The sequence of the T-oligonucleotide is unique to the binding agent to which it is conjugated. For a set of T-oligonucleotides there is a corresponding FRET-oligonucleotide which can hybridise to multiple T-oligonucleotides in the set, to form multiple pairs. The sequences of the T-oligonucleotide and the FRET-oligonucleotide are such that the pairs that are formed undergo dissociation and reassociation and this dissociation and reassociation between each different pair generates a fluorescent kinetic profile that is unique within the set to that pair. Because the fluorescent kinetic profile is unique within the set to that pair, the observation of a particular fluorescent kinetic profile at one or more pixel indicates the presence of a particular T-oligonucleotide at that location in the sample (and hence the presence of the binding agent to which that T-oligonucleotide is conjugated, and ultimately the target molecule to which the binding agent binds, at that location in the sample). In other words, the observation of a particular fluorescent kinetic profile at one or more pixel indicates the presence of the corresponding target molecule at that location in the sample.

The term “fluorescent kinetic profile” as used herein describes the pattern of fluorescence emission that results from the dissociation and reassociation of any pair of a T-oligonucleotide and its corresponding FRET-oligonucleotide over time. The fluorescence from the emitter molecule at the emitter molecules emission wavelength (which is a direct result of the pair's dissociation and reassociation over time), can be expressed as fluorescence intensity over time (e.g. plotted as set out in FIG. 3A-FIG. 3D, FIG. 4A-FIG. 4B, FIG. 5A-FIG. 5D, FIG. 12A-FIG. 12B, and FIG. 13). The dissociation and reassociation of each pair is a stochastic process, however, so it will be appreciated that an individual observation of a pair's fluorescent kinetic profile will not necessarily look identical every time, and each individual observation of a given pair's fluorescent kinetic profile may look different when represented on a plot. There are, however, discernible properties or characteristics of a pair's fluorescent kinetic profile that are consistent between each individual observation of their fluorescent kinetic profile. These consistent properties or characteristics can be used to describe and/or define a pair's fluorescent kinetic profile. These properties or characteristics include the metrics that are discussed elsewhere herein.

A single observed fluorescent kinetic profile therefore can be described by reference to one or more consistent property or characteristic (e.g. one or more of the metrics referred to elsewhere herein). A pair's single observed fluorescent kinetic profile may thus not necessarily look identical each time that it is observed, but a given pair's fluorescent kinetic profile can be described as being distinct, and as being unique to a pair within each set (or uniquely discernible to a pair within each set) or unique within a group of sets (or uniquely discernible to a pair within a group of sets) because its consistent properties or characteristics allow the identification of a pair within each set.

Because a single FRET-oligonucleotide can form multiple pairs, each of which generates a distinct fluorescent kinetic profile, with each fluorescent kinetic profile being specific to and hence indicative of the T-oligonucleotide with which it has formed a pair, the current method readily accommodates, and in fact is designed for, effective multiplexing. At the observation stage, a fluorescent kinetic profile is observed, and may be recorded, at one or more pixel. Where a fluorescent kinetic profile is observed, an identity can be assigned to each pixel, based on the fluorescent kinetic profile. In general this will be achieved by calculating a distinguishable metric of the fluorescent kinetic profile and optionally assigning an identity to one or more pixels in the observed sample based on the metric of the fluorescent kinetic profile of the sample at that pixel. Since each different T-oligonucleotide-FRET-oligonucleotide pair generates its own fluorescent kinetic profile, even with a single FRET oligonucleotide, multiplexing can be achieved. In fact using a single FRET-oligonucleotide is advantageous since multiple fluorescent kinetic profiles can be observed and optionally recorded in the same spectral channel.

In the methods of the invention a set of binding agent-T-oligonucleotide conjugates will in general contain a plurality of binding agents having different specificities. There may, for example be 2 or more, 3 or more, 4 or more, 5 or more, 6 or more, 7 or more, 8 or more, 9 or more or 10 or more 11 or more, 12 or more, 13 or more, 14 or more 15 or more 16 or more 17 or more 18 or more, 19 or more, 20 or more, 21 or more, 22 or more, 23 or more 24 or more or 25 or more binding agents having different specificities in a set, with each binding agent being conjugated to a T-oligonucleotide and with the sequence of the T-oligonucleotide being unique to the binding agent to which it is conjugated. There may, for example be up to 3, up to 4, up to 5, up to 6, up to 7, up to 8, up to 9, up to 10, up to 11, up to 12, up to 13, up to 14, up to 15, up to 16, up to 17, up to 18, up to 19, up to 20, up to 21, up to 22, up to 23, up to 24, up to 25 binding agents having different specificities in a set, with each binding agent being conjugated to a T-oligonucleotide and with the sequence of the T-oligonucleotide being unique to the binding agent to which it is conjugated. Suitable ranges include 2-25, 3-24, 4-23, 5-22, 6-21, 7-20, 8-19, 9-20, 10-19, 11-18, 12-17, 13-16, 14-15 binding agents having different specificities in a set, with each binding agent being conjugated to a T-oligonucleotide and with the sequence of the T-oligonucleotide being unique to the binding agent to which it is conjugated.

A larger number of different fluorescent kinetic profiles can be achieved by increasing the number of different FRET-oligonucleotides that are used. This is because for any given FRET-oligonucleotide (i.e. a FRET-oligonucleotide having a given sequence) there may be a finite number of different fluorescent kinetic profiles that can be generated, even using a set of T-oligonucleotides having very diverse sequences. Increasing the number of different FRET-oligonucleotides that are used in the method, with each FRET-oligonucleotide having a corresponding set of T oligonucleotides, can increase the number of different fluorescent kinetic profiles that can be generated. Therefore a greater number of different binding agents can be used in the method of the invention, and a greater number of target molecules can be detected, in a single experiment.

As discussed above, the average period of time between each fluorescence emission (which can be conceptualised as “blinking”) is one component of the fluorescent kinetic profile, and is a metric that can be derived from the observed fluorescent kinetic profile. A short average period of time between each fluorescence emission at the emitter molecule's emission wavelength is seen when the T-oligonucleotide and the FRET-oligonucleotide hybridise or associate relatively quickly, i.e. when the duration of time the two components spend dissociated from each other is short (low off-time). For example the profiles of FIG. 3B and FIG. 3C represent pairs with a lower off time than the profile of FIG. 3A. A high frequency or rate of “blinking” is seen when the T-oligonucleotide and the FRET-oligonucleotide hybridise or associate relatively quickly, but also dissociate relatively quickly. It may be possible to achieve a certain range of blinking kinetics profiles using a single FRET-oligonucleotide and one set of T-oligonucleotides. Using a second FRET-oligonucleotide and corresponding set of T-oligonucleotides may allow for distinct fluorescent kinetic profiles to be generated to those that arise from the first FRET-oligonucleotide and corresponding set of T-oligonucleotides (e.g. they may give rise to a range that is overlapping or non-overlapping with the first range).

One aspect of the FRET-oligonucleotide that can be changed readily (e.g. in order to increase the number of different fluorescent kinetic profiles) is the length of the FRET oligonucleotide. Longer oligonucleotides have the potential to hybridise more strongly to their pairs than shorter oligonucleotides and thereby increase the average amount of time that it takes for the FRET-oligonucleotide and T-oligonucleotide to dissociate. This can be expressed in terms of the K_{off} of a pair (e.g. a reduced K_{off} value is seen for a pair that hybridises more strongly than for a pair that hybridises more weakly). In turn, therefore a longer FRET-oligonucleotide may contribute to pairs that generate fluorescent kinetic profiles that have a longer average duration of fluorescence emission (i.e. a higher on time) than pairs in which the FRET-oligonucleotide is shorter. For example, the profile in FIG. 3B is a representation of a fluorescent kinetic profile of a pair with a higher on time than the profiles of FIG. 3A and FIG. 3C).

Having one longer FRET-oligonucleotide and one shorter FRET-oligonucleotide is just one example of an approach to give rise to an increased range of blinking kinetic profiles. Other examples include varying the base content (e.g. a GC rich FRET oligonucleotide will tend to have the potential to hybridise more strongly to its pairs than an AT rich oligonucleotide and a GC rich FRET oligonucleotide thereby increases the average amount of time that it takes for the FRET-oligonucleotide and T-oligonucleotide to dissociate. This can be expressed in terms of the K_{off} of a pair (e.g. a reduced K_{off} value is seen for a pair that hybridises more strongly than for a pair that hybridises more weakly).

Irrespective of the precise way in which the multiple fluorescent kinetic profiles are generated, it will be understood that by producing multiple sets of T-oligonucleotides and corresponding FRET oligonucleotides, the number of different fluorescent kinetic profiles that can be generated is increased. FIG. 3A-FIG. 3C show this in a simplified format. The profiles of FIG. 3B and FIG. 3C represent pairs with a lower off time than the profile of FIG. 3A, and the profiles of FIG. 3A and FIG. 3C represent pairs with a lower on time than the profile of FIG. 3B. It will be understood that large numbers of different fluorescent kinetic profiles can be generated, having for example different on time and off time values. This in turn increases the potential for multiplexing. In other words a greater degree of multiplexing is possible by using multiple sets of T-oligonucleotides and corresponding FRET-oligonucleotide than using a single set of T-oligonucleotides and corresponding FRET oligonucleotide. As discussed in more detail below, exemplary and non-limiting metrics that can be derived or calculated from the fluorescent kinetic profile may include (i) the average period of time between each fluorescence emission (also referred to as the off-time), (ii) the average duration of the fluorescence emission (also referred to as the on-time), and (iii) the rate of occurrences of fluorescence emission.

To illustrate the above, a given set of binding agent-T-oligonucleotide conjugates may, for example comprise 10 T-oligonucleotides (T-oligonucleotides 1 to 10). FRET-oligonucleotide A may for example reversibly hybridise to these 10 T-oligonucleotides and generate 10 different fluorescent kinetic profiles (profiles 1 to 10). By adding a further set of binding agent-T-oligonucleotide conjugates, e.g. comprising 10 T-oligonucleotides (T-oligonucleotides 11 to 20) and the corresponding FRET-oligonucleotide (FRET-oligonucleotide B), a further 10 different fluorescent kinetic profiles (profiles 11 to 20) can be used. If FRET-oligonucleotide A and FRET-oligonucleotide B emit fluorescence that can be observed in the same channel (e.g. if they contain emitter molecules that have an emission spectrum that is overlapping, or if they contain the same emitter molecule), then all of the 20 different fluorescent kinetic profiles can be observed in a single spectral channel. If FRET-oligonucleotide A and FRET-oligonucleotide B emit fluorescence that requires more than one spectral channel to observe the fluorescent kinetic profiles or if they may be observed in different channels or are optimally observed in different channels (e.g. if they contain emitter molecules that have emission spectrums that are not overlapping, or if they contain emitter molecules that have emission spectrums with different emission maximum values, or different emitter molecules), then more than one spectral channel may be used to observe the fluorescent kinetic profiles.

Where more than one FRET-oligonucleotide and corresponding T-oligonucleotides are used, this is referred to herein as using a group of sets of T-oligonucleotides. Even when a group of sets of T-oligonucleotides is used, the sequence of each T-oligonucleotide that is present is unique to the binding agent to which it is conjugated. Further, the dissociation and reassociation between each different pair generates a fluorescent kinetic profile that is (i) unique within that set to that pair, and (ii) unique within the group of sets to that pair. Furthermore, in some embodiments, each T-oligonucleotide hybridises only to its corresponding FRET-oligonucleotide and does not hybridise to a FRET oligonucleotide(s) of any other set in the group of sets. Cross hybridisation should be avoided, so that the correlation between the fluorescent kinetic profile, the T-oligonucleotide and the binding agent is preserved.

A further level of multiplexing can be achieved by increasing the number of different channels in which the fluorescent kinetic signal is observed, e.g. using FRET-oligonucleotides with fluorophores having different emission spectra, to generate fluorescent kinetic profiles as fluorescence emissions that can be detected in different channels. In such cases, each fluorescent kinetic profile that is generated can be different, but using multiple channels means that a particular fluorescent kinetic profile can be duplicated between the groups of sets. In other words, two or more pairs that generate very similar or even identical fluorescent kinetic profiles can be used, if those two or more pairs that generate the similar or even identical fluorescent kinetic profiles generate fluorescent kinetic profiles as fluorescence emissions that are observed in different channels.

With this type of multiplexing, each T-oligonucleotide will still have a different sequence, that is unique to the binding agent to which it is conjugated, and each different pair in a set generates a fluorescent kinetic profile that is unique within that set to that pair, however using FRET-oligonucleotides that generate fluorescent kinetic profiles as fluorescence emissions that can be detected in different channels further improves the potential for multiplexing.

To illustrate the above, a given set of binding agent-T-oligonucleotide conjugates may, for example comprise 10 T-oligonucleotides (T-oligonucleotide 1 to 10). FRET-oligonucleotide A (e.g. which fluoresces in “red”) may for example reversibly hybridise to these 10 T-oligonucleotides and generate 10 different fluorescent kinetic profiles (profiles 1 to 10). By adding a further set of binding agent-T-oligonucleotide conjugates, e.g. comprising 10 T-oligonucleotides (T-oligonucleotides 11 to 20) and the corresponding FRET-oligonucleotide (FRET-oligonucleotide B (e.g. which also fluoresces in “red”)), a further 10 different fluorescent kinetic profiles (profiles 11 to 20) can be used. As FRET-oligonucleotide A and FRET-oligonucleotide B contain emitter molecules that have an emission spectrum that is overlapping (e.g. if they contain the same emitter molecule) their fluorescence can be observed in the same channel (referred to here as “red”), then all of the 20 different fluorescent kinetic profiles can be observed in a single channel Here, binding agent-T-oligonucleotide conjugates comprising T-oligonucleotides 1 to 10 are one set, and binding agent-T-oligonucleotide conjugates comprising T-oligonucleotides 11 to 20 are a second set, and binding agent-T-oligonucleotide conjugates comprising T-oligonucleotides 1 to 20 are therefore a “group of sets”.

A further group of sets of binding agent-T-oligonucleotide conjugates may be used, for example a further set of binding agent-T-oligonucleotide conjugates may, for example comprise 10 T-oligonucleotides (T-oligonucleotide 21 to 30). FRET-oligonucleotide C (e.g. which fluoresces in “green”) may for example reversibly hybridise to these 10 T-oligonucleotides and generate 10 different fluorescent kinetic profiles (profiles 21 to 30). A further set of binding agent-T-oligonucleotide conjugates, e.g. comprising 10 T-oligonucleotides (T-oligonucleotides 31 to 40) and the corresponding FRET-oligonucleotide (FRET-oligonucleotide D (e.g. which also fluoresces in “green”)), may generate a further 10 different fluorescent kinetic profiles (profiles 31 to 40). As FRET-oligonucleotide C and FRET-oligonucleotide D contain emitter molecules that have an emission spectrum that is overlapping (e.g. they contain the same emitter molecule) their fluorescence can be observed in the same channel (referred to here as “green”), thus all of the 20 different fluorescent kinetic profiles can be observed in a single channel. Here, binding agent-T-oligonucleotide conjugates comprising T-oligonucleotides 21 to 30 are one set, and binding agent-T-oligonucleotide conjugates comprising T-oligonucleotides 31 to 40 are a second set, and binding agent-T-oligonucleotide conjugates comprising T-oligonucleotides 21 to 40 are therefore a “group of sets”.

In this example therefore these two groups of sets (i.e. “multiple groups of sets”) can be used together in a multiplexed fluorescence microscopy method in which 40 binding agents may be used. Each binding agent-T-oligonucleotide comprises a T-oligonucleotide with a sequence that is unique to the binding agent to which it is conjugated. Further, the dissociation and reassociation between each different pair generates a fluorescent kinetic profile that is (i) unique within each set to that pair, and (ii) unique within each group of sets to that pair. The dissociation and reassociation between each different pair may generate a fluorescent kinetic profile that is also unique within the multiple groups of sets to that pair, but this is not necessary, since two similar or identical fluorescent kinetic profiles can be used, if the two fluorescent kinetic profiles are generated by FRET-oligonucleotides having different emitter molecules (e.g. emitter molecules with emission spectra that can be observed in different channels, e.g. different fluorescence emission spectra).

In the theoretical example above therefore, the oligonucleotides may be designed to generate 40 different fluorescent kinetic profiles. Oligonucleotides that generate fewer than 40 different fluorescent kinetic profiles can be used if, for example a pair using FRET-oligonucleotide A and one T-oligonucleotide and FRET-oligonucleotide C and another T-oligonucleotide both give rise to similar or identical fluorescent kinetic profiles. This is because the fluorescent kinetic profiles obtained using FRET-oligonucleotide A and those obtained using FRET-oligonucleotide C are detected in different channels.

The above therefore demonstrates how further multiplexing may be achieved using the present invention. It is not necessary to build up the multiplexing exactly as set out above. It is possible, for example to use multiple sets of binding agent-T-oligonucleotide conjugates, wherein the emission spectrum of the emitter molecules in the corresponding FRET-oligonucleotides for each set of binding agent-T-oligonucleotide can be detected in a channel that is specific to that set and the step of observing the fluorescent kinetic profile of the sample comprises observing the fluorescent kinetic profile of the sample in a channel for each set.

For example a first set of binding agent-T-oligonucleotide conjugates may, for example comprise 10 T-oligonucleotides (T-oligonucleotide 1 to 10). FRET-oligonucleotide A (red) may for example reversibly hybridise to these 10 T-oligonucleotides and generate 10 different fluorescent kinetic profiles (profiles 1 to 10). A second set of binding agent-T-oligonucleotide conjugates, e.g. comprising 10 T-oligonucleotides (T-oligonucleotides 11 to 20) and the corresponding FRET-oligonucleotide (FRET-oligonucleotide B (green)), to generate 10 different fluorescent kinetic profiles (profiles 11 to 20). Each binding agent-T-oligonucleotide comprises a T-oligonucleotide with a sequence that is unique to the binding agent to which it is conjugated. The dissociation and reassociation between each different pair generates a fluorescent kinetic profile that is unique within each set to that pair. The fluorescent kinetic profile may be, but does not have to be, unique within the multiple sets to that pair. The dissociation and reassociation between each different pair may generate a fluorescent kinetic profile that is also unique within the multiple sets to that pair, but this is not necessary, since two similar or identical fluorescent kinetic profiles can be used, if the two fluorescent kinetic profiles are generated by FRET-oligonucleotides having different emitter molecules (e.g. emitter molecules with emission spectra that can be observed in different channels, e.g. fluorescence emission spectra).

In another example, one or multiple sets of binding agent-T-oligonucleotide conjugates, wherein the emission spectrum of the emitter molecules in the corresponding FRET-oligonucleotide for each set of binding agent-T-oligonucleotide can be detected in a channel that is specific to that set is used, together with one or multiple groups of sets of binding agent-T-oligonucleotide conjugates wherein the emission spectrum of the emitter molecules in the corresponding FRET-oligonucleotides for each group of sets of binding agent-T-oligonucleotide can be detected in a channel that is specific to that group of sets. In other words, a (e.g. one) set of binding agent-T-oligonucleotide conjugates may be used with a (e.g. one) group of sets of binding agent-T-oligonucleotide conjugates (wherein the emission spectrum of the emitter molecules in the corresponding FRET-oligonucleotides for each group of sets of binding agent-T-oligonucleotide can be detected in a channel that is specific to that group). A (e.g. one) set of binding agent-T-oligonucleotide conjugates, may be used together with multiple groups of sets of binding agent-T-oligonucleotide conjugates (wherein the/emission spectrum of the emitter molecules in the corresponding FRET-oligonucleotides for each group of sets of binding agent-T-oligonucleotide can be detected in a channel that is specific to that group). Multiple sets of binding agent-T-oligonucleotide conjugates, wherein the emission spectrum of the emitter molecules in the corresponding FRET-oligonucleotide for each set of binding agent-T-oligonucleotide can be detected in a channel that is specific to that set may be used, together with a (e.g. one) group of sets of binding agent-T-oligonucleotide conjugates wherein the emission spectrum of the emitter molecules in the corresponding FRET-oligonucleotides for each group of sets of binding agent-T-oligonucleotide can be detected in a channel that is specific to that group. Multiple sets of binding agent-T-oligonucleotide conjugates, wherein the emission spectrum of the emitter molecules in the corresponding FRET-oligonucleotide for each set of binding agent-T-oligonucleotide can be detected in a channel that is specific to that set may be used, together with multiple groups of sets of binding agent-T-oligonucleotide conjugates wherein the emission spectrum of the emitter molecules in the corresponding FRET-oligonucleotides for each group of sets of binding agent-T-oligonucleotide can be detected in a channel that is specific to that group.

Certain examples of how multiplexing may be achieved according to the present invention include those summarised in Table 1:

| Multiplexing | T- | FRET | Multiplexing | How |
|---|---|---|---|---|
| level | oligonucleotide | oligonucleotide | level | observed |
| 1 | A set of T- | One | One fluorescent kinetic | single |
| | oligonucleotides | | profile per T- | channel |
| | having different | | oligonucleotide. | |
| | sequences | | | |
| Example for | T1-T10 | FRET A | 10 FKPs | |
| level 1 | | | | |
| 2 | A group of sets of | One per set of T- | One fluorescent kinetic | single |
| | T-oligonucleotides | oligonucleotides | profile per T- | channel or |
| | | | oligonucleotide. | multiple |
| | | | Multiple FRET- | channel |
| | | | oligonucleotides | |
| | | | increases the number of | |
| | | | different fluorescent | |
| | | | kinetic profiles that are | |
| | | | generated. | |
| Example for | T1-T10 | FRET A | 20 FKPs | |
| level 2 | T11-20 | FRET B | | |
| 3 | Multiple groups | One per set of T- | One fluorescent kinetic | One channel per |
| | of sets of T-oligonucleotides | oligonucleotides. | profile per T-oligonucleotide | group of sets |
| | | The FRET- | Use of multiple FRET- | |
| | | oligonucleotides | oligonucleotide | |
| | | for each group of | increases the number of | |
| | | sets have the same | different fluorescent | |
| | | “colour” | kinetic profiles that are | |
| | | | generated | |
| | | | Possible to use pairs | |
| | | | that generate similar or | |
| | | | identical profiles if the | |
| | | | FRET oligos emit in a | |
| | | | different “colour” | |
| Example for | T1-T10 | FRET A | 20 “red” FKPs- this is | |
| level 3 | | (e.g. “red”) | one “group of sets” | |
| | T11-20 | FRET B | 20 “green”FKPs - this | |
| | | (e.g. “red”) | is one “group of sets” | |
| | T21-30 | FRET C | | |
| | | (e.g. “green”) | | |
| | T31-40 | FRET D | | |
| | | (e.g. “green”) | | |
| Alternative | T1-T10 | FRET A | 10 “red” FKPs- this is | |
| example | | (e.g. “red”) | one “set” | |
| One set and | T21-30 | FRET C | 20 “green”FKPs - this | |
| one group of | | (e.g. ”green”) | is one “group of sets” | |
| sets | T31-40 | FRET D | | |
| | | (e.g. “green”) | | |
| Alternative | T1-T10 | FRET A | 10 “red” FKPs- this is | |
| example | | (e.g. “red”) | one “set” | |
| multiple sets | T21-30 | FRET C | 10 “green”FKPs - this | |
| and one | | (e.g. “green”) | is one “set” | |
| group of sets | T41-50 | FRET E | 20 “blue”FKPs - this is | |
| | | (e.g.“blue”) | one “group of sets” | |
| | T51-60 | FRET F | | |
| | | (e.g. “blue”) | | |
| Alternative | T1-T10 | FRET A | 10 “red” FKPs- this is | |
| example | | (e.g. “red”) | one “set” | |
| multiple sets | T21-30 | FRET C | 10 “green”FKPs - this | |
| and multiple | | (e.g. ”green”) | is one “set” | |
| group of sets | T41-50 | FRET E | 20 “blue”FKPs - this is | |
| | | (e.g. “blue”) | one “group of sets” | |
| | T51-60 | FRET F | | |
| | | (e.g. “blue”) | | |
| | T61-70 | FRET G | | |
| | | (e.g. “yellow”) | 20 “yellow” FKPs - | |
| | T71-80 | FRET H | this is one “group of | |
| | | (e.g. “yellow”) | sets” | |

### Sample

The sample will in general comprise one or more cells (e.g. a population of cells), or may comprise a cell lysate from a cell or a population of cells. The sample may contain prokaryotic or eukaryotic cells, e.g. animal, plant, yeast, bacterial or other cells. The cells are preferably animal cells. They may be of animal original and may in particular be of mammalian origin. They may be primary cells or may be cells that are a cell line, e.g. a mammalian cell line. The sample may contain cells that are from an animal model of a disease or a human or animal patient with a disease. The sample may be a biological sample, e.g. from an animal (preferably a mammal such as a human or an experimental animal e g a mouse or rat) and the sample may e.g. by blood, sputum, lymph, mucous, stool, urine and the like. The sample may be a tissue sample such as a tissue section. The sample may be an environmental sample such as a water sample, an air sample, a food sample, and the like.

### Fixation of the Sample

The methods of the invention are carried out on a fixed sample, which may be a chemically fixed sample. Fixation of samples for fluorescence microscopy is well known and is carried out in order to preserve the sample (e.g. to maintain cell morphology, in a state that is structurally close to its state when living). Fixation may also prevent the autolysis and necrosis of excised tissues, if used. It may also preserve antigenicity, and allow the components used in the methods to more readily access the internal portions of the cell.

The methods of the invention may further comprise one or more steps of fixing the sample, e.g. to preserve it. This may be carried out before step a) of the method as described herein. Further optional fixation steps may be carried out after contacting the fixed sample with a set of binding agent-T-oligonucleotide conjugates to allow the binding agents to bind to any binding partners present in the sample, and before contacting the sample and any bound binding agents resulting from step a) with a FRET-oligonucleotide. Carrying out a further fixation step at this stage of the method is advantageous to further immobilise the bound binding agents on the sample. The binding agents may bind to their target molecules using e.g. electrostatic bonds, but the further processing steps of the sample may be improved if fixation is performed. The improvement may arise as a result of the fact that the fixation may cause stronger bonds to be created between the bound binding agents and the target molecules in the sample, e.g. crosslinking the bound binding agents and the target molecules in the sample with covalent bonds.

Fixation, such as chemical fixation is well known in the art.

Examples of suitable ways to carry out chemical fixation include, for example using crosslinking fixatives (e.g. to create covalent chemical bonds between protein in samples), such as those that are based on aldehyde, or oxidising crosslinking fixatives. Examples of aldehyde based fixatives include formaldehyde, glutaraldehyde, glyoxal, paraformaldehyde. Oxidising crosslinking fixatives for example osmium tetroxide, chromic acid, potassium dichromate, potassium permanganate. Precipitating fixatives may alternatively be used, for example acetone, ethanol, methanol. Alternative agents include Hepes-glutamic acid buffer-mediated organic solvent protection effect (HOPE) fixative, mercurial such as B-5 and Zenker's fixative, and picrates. Preferably aldehyde crosslinking fixatives are used.

### Permeabilization of the Sample

Permeabilisation of the sample may also be carried out. In some embodiments therefore the method comprises one or more steps of permeabilising the sample, e.g. to allow molecules used in the method to access the interior of the cell. This may be carried out before step a) of the method as described herein and before, after or simultaneously with any fixing step. Suitable permeabilisation agents and protocols are known in the art and include the use of agents such as saponin, Triton-X and Tween-20. Suitable protocols are described for example in Jamur M. C., Oliver C. ((2010) Permeabilization of Cell Membranes. In: Oliver C., Jamur M. (eds) Immunocytochemical Methods and Protocols. Methods in Molecular Biology (Methods and Protocols), vol 588. Humana Press), or as described in https://www.abcam.com/ps/pdf/protocols/fixation_permeabilization.pdf

### Further Sample Processing

In step a) a fixed sample is contacted with a set of binding agent-T-oligonucleotide conjugates to allow the binding agents to bind to any binding partners present in the sample. In general the sample is fixed and is therefore attached to a solid support of any suitable time for fluorescent microscopy. The contacting step may be carried out in any appropriate manner to enable the set of binding agent-T-oligonucleotide conjugates to bind to any binding partners present in the sample. This will in general be by applying a solution containing the set of binding agent-T-oligonucleotide conjugates to the solid support to which the sample is attached. The binding agent-T-oligonucleotide conjugates may be present in the solution at a concentration of e.g. 5-50 ug/ml (or 1-500, 2-250 ug/ml).

Suitable conditions include incubation at about 4° C., or about 15-25° C., e.g. in a buffer solution containing the binding agent-T-oligonucleotide conjugates and blocking reagents, such as BSA or salmon sperm DNA.

The contacting may continue for any appropriate length of time to allow the set of binding agent-T-oligonucleotide conjugates to bind to any binding partners. Exemplary time periods include 10 minutes to 2 hours, e.g. 30 minutes to 1 hour.

An advantage of the present method as described herein is that washing steps are not required, and background fluorescence levels are generally not high, e.g. compared to DNA-PAINT methods discussed above. This means that washing steps to remove unbound components are not in general required in order to obtain good quality data. However, a step of removing unbound binding agent-T-oligonucleotide conjugates after step a may be carried out. This may be carried in order to further reduce background fluorescence levels, because the FRET-oligonucleotide may bind to unbound binding agent-T-oligonucleotide conjugates which in turn may give rise to background fluorescence.

The washing step may be carried out e.g. by removing the solution containing the binding agent-T-oligonucleotide conjugates and replacing this with one or more changes of a liquid which does not contain binding agent-T-oligonucleotide conjugates, e.g. an appropriate buffer.

The step of contacting the sample and any bound binding agents resulting from step a with a FRET-oligonucleotide may be carried out directly after step a, or after one or more washing step. The contacting step may be carried out in any appropriate manner to enable the FRET-oligonucleotide to hybridise to one or more T-oligonucleotides. In this way multiple pairs are formed. This will in general be carried out by applying a solution containing the FRET-oligonucleotide to the sample and any bound binding agents resulting from step a. The FRET-oligonucleotide may be present in the solution at a concentration of e.g. 1 pM to 1 uM (or 10 pM to 500 nM, 20 pM to 250 nM, 50 pM to 100 nM, 100 pM to 50 nM, 200 pM to 25 nM, 500 pM to 1 nM).

The steps of illuminating the sample at a wavelength to cause excitation of the emitter molecule; and observing the kinetic fluorescent profile of the sample at one or more pixels over time in general occur once the sample has been contacted with the FRET-oligonucleotide. The illumination and observation steps thus occur while the sample is in contact with the FRET-oligonucleotide and may continue as long as the sample is in contact with the FRET-oligonucleotide, or for a suitable period to capture and optionally record the fluorescent kinetic profile.

### Oligonucleotide

An oligonucleotide, such as a T-oligonucleotide or a FRET oligonucleotide, is a linear polymeric molecule comprising linked units, wherein the units comprise nucleobases or functional equivalents thereof. The nucleobases may be naturally occurring nucleobases such as adenine, thymine, guanine, cytosine, uracil, inosine, pseudouridine, dihydrouridine, 7-methylguanosine (and are preferably selected from adenine, thymine, guanine, cytosine, and uracil) and their derivatives, or non-naturally occurring nucleobases such as isocytosine, isoguanine. The units comprising the nucleobases may be nucleosides (nucleobase and a sugar group, e.g. a five carbon sugar, preferably ribose or deoxyribose) but are preferably nucleotides (nucleoside and a phosphate). Nucleotides are the monomeric units of DNA and RNA and the oligonucleotides used in the methods of the invention preferable comprise nucleotides, e.g. deoxyribonucleotides or ribonucleotides, or analogs thereof.

Preferably the oligonucleotide does not include any fluorescent nucleobases. In preferred embodiments, the oligonucleotide is or comprises DNA.

Well known examples of oligonucleotides include those that are or comprise DNA, RNA or other nucleic acid-like structures. They may have a phosphate-sugar backbone or a backbone comprising non-phosphate-sugar moieties (e.g. peptide nucleic acid (PNA) and morpholino). Phosphate-sugar backbone analogues include PNA, morpholino and locked nucleic acid (LNA), as well as glycol nucleic acid (GNA), threose nucleic acid (TNA) and hexitol nucleic acids (HNA). Each of these is distinguished from naturally occurring DNA or RNA by changes to the backbone of the molecule.

The invention capitalises on oligonucleotides' abilities as single stranded molecules to hybridise with complementary or partially complementary oligonucleotides to form double stranded pairs. Two fully complementary or partially complementary oligonucleotides can bind to each other to form a double stranded molecule, based on complementary base pairing, as is well known in the art.

In the methods of present invention, the T-oligonucleotides and their corresponding FRET-oligonucleotide are designed to have sequences that are in general only partially complementary, so that the oligonucleotide pairs that are formed between the T-oligonucleotides and their corresponding FRET-oligonucleotide are in double stranded form only transiently. Each pair undergoes a process of association and dissociation over a period of time. The transient nature of the hybridisation is reflected in a fluorescent kinetic profile because the FRET-oligonucleotide contains a fluorescence emitter molecule and a quenching entity. When the FRET-oligonucleotide is in solution, the proximity of the quenching entity to the fluorescence emitter molecule means that upon illumination with light of an appropriate wavelength the fluorescence emitted from the emitter molecule is quenched by the quenching entity and is not detected at the emitter molecule's emission wavelength. When the FRET-oligonucleotide is hybridised to a T-oligonucleotide this constrains the structure of the FRET-oligonucleotide so that the quenching entity and the fluorescence emitter molecule are physically separated so that upon illumination with light of an appropriate wavelength the fluorescence emitted from the emitter molecule is not quenched by the quenching entity and may be observed at the emitter molecule's emission wavelength. Association between the two members of each pair is therefore accompanied with fluorescence at the emitter molecule's emission wavelength and dissociation with an absence of fluorescence at the emitter molecule's emission wavelength.

In each set of T-oligonucleotides the members of the set will have different sequences and will dissociate and reassociate with the corresponding FRET-oligonucleotide in a manner that is dependent on their sequence. The hybridisation kinetics of the two oligonucleotides in each pair therefore determines the fluorescent kinetic profile that may be observed.

An oligonucleotide, when not bound to its complementary or partially complementary pair, may be single-stranded, and may be without stable secondary structure. In preferred embodiments in the single-stranded form the oligonucleotides as used herein do not form stable secondary structure, such as hairpin loops. This provides advantages in the present invention, since oligonucleotides that do not form such stable secondary structure can form pairs more readily and therefore more rapidly, making them more amenable to imaging.

### T-Oligonucleotides

A T-oligonucleotide has a sequence that is specific to the binding agent to which it is conjugated. A T-oligonucleotide is also capable of hybridising to its corresponding FRET oligonucleotide. The sequence of an oligonucleotide is determined by the nature and the order of the units that make it up. Standard terminology may be used to define its sequence (e.g. referring to the base sequence of the molecule).

A T-oligonucleotide is generally 8-35 nucleotides or equivalent units in length, with one nucleotide unit being the size of a standard DNA nucleotide unit. The T-oligonucleotide may alternatively be 9-30, 10-29, 11-28, 12-27, 13-26, 14-25, 15-24, 16-23, 17-22, 18-21, or 10-19, 11-18, 12-17, 13-18, 14-17, 15-16 nucleotide units in length (e.g. up to 35, 34, 33, 32, 31 30, 29, 28, 27, 26, 25, 24, 23, 22, 21, 20, 19, 18, 17, 16, 15, 14, 13, 12, 11, 10 or at least 8, 9, 10, 11, 12, 13, 14, 15, 16, 17, 18, 19, 20, 21, 22, 23, 24, 25, 26, 27, 28, 29, 30, 31, 32, 33, 34 nucleotide units in length).

The T-oligonucleotide is in general capable of hybridising to its corresponding FRET-oligonucleotide and does not in general hybridise to other T-oligonucleotides in the set or to any other T-oligonucleotides in the group of sets, if used. In general each T-oligonucleotide hybridises to its corresponding FRET-oligonucleotide and not to FRET-oligonucleotides that are used in the method other than its corresponding FRET-oligonucleotide.

A T-oligonucleotide and its corresponding FRET-oligonucleotide will contain nucleotides or functional equivalents thereof that are complementary. When complementary bases are present in two oligonucleotide strands, hydrogen bonding between the complementary bases can occur. Hybridization is a non-covalent, sequence-specific interaction between two oligonucleotide strands to form a single complex of two strands.

Two complementary strands with one or more mismatches may also bind to each other but will do so less readily, because the mismatches make binding between them less energetically favorable. The changes to the binding properties will depend on at least (i) the length of the oligonucleotides (e.g. over the overlapping region) (ii) the composition of the sequences, (ii) the degree of complementarity (e.g. over the overlapping region), (iii) the location of mismatches within the two strands (adjacent mismatches will in general have more of an effect on the stability of the interaction between the two strands than mismatches which are not adjacent), (iv) the nature of the complementary pairs (for example the pairs of nucleic bases A=T form less stable bonds than the GC pairs).

As discussed elsewhere herein, the T-oligonucleotides are generally 8-35 or 9-20 oligonucleotide units in length and the FRET-oligonucleotides are of a length that exceeds the Forster Radius of the FRET-oligonucleotide emitter molecule, and/or (ii) at least 12 nucleotide units in length.

In general, the strength with which two oligonucleotides hybridise will also depend on the degree of complementarity between the two strands (i.e. the number of the bases that are complementary in comparison to the total length of each oligonucleotide or in comparison to the length of overlap between the two oligonucleotides where the sequences are not the same length). Some degree of sequence complementarity is required for hybridisation to occur.

There is generally a range of between 0-40%, 10-40% or 20-40% mismatches between a T-oligonucleotide and its corresponding FRET-oligonucleotide, as compared to the maximum number of base pairs that could be formed in such a pair. The T-oligonucleotide is generally 8 to 35 nucleotide units in length and the corresponding FRET-oligonucleotide is generally of a length that exceeds the Forster Radius of the FRET-oligonucleotide emitter molecule, and/or is (ii) at least 12 nucleotide units in length. The percentage of mismatches is defined as the percentage of mismatches as compared to the maximum number of base pairs that could be formed, in view of the fact that the two oligonucleotides are not always the same length. In other words, where the T-oligonucleotide is 10 nucleotide units in length and the corresponding FRET-oligonucleotide is 15 nucleotide units in length, the maximum number of base pairs that could be formed in this pair would be 10, and therefore 0-40% mismatches equates in this pair to 0-4 mismatches in the overlapping 10 nucleotide unit stretch and 20-40% mismatches equates in this pair to 2-4 mismatches in the overlapping 10 nucleotide unit stretch. This is represented in the Table below in which exemplary numbers of mismatches for each pair to give 20-40% mismatches are shown, together with the maximum number of base pairs that could be formed for each pair, (shown in brackets). In another example when the T-oligonucleotide is 12 nucleotide units long and the FRET-oligonucleotide is also 12 nucleotide units long, the maximum number of base pairs that could be formed in this pair is 12, and the number of mismatches that represents 20-40% of this is 2-4.

The number of mismatches that equates to 20-40% for certain preferred combinations of T-oligonucleotide and FRET-oligonucleotide is set out in Table 2 below.

TABLE 2

| | | FRET-oligonucleotide length (nucleotide units) | | | | | | | | |
|---|---|---|---|---|---|---|---|---|---|---|
| | | 12 | 13 | 14 | 15 | 16 | 17 | 18 | 19 | 20 |
| T- | 8 | 1-3 | 1-3 | 1-3 | 1-3 | 1-3 | 1-3 | 1-3 | 1-3 | 1-3 |
| oligonucleotide | | (8) | (8) | (8) | (8) | (8) | (8) | (8) | (8) | (8) |
| length | 9 | 1-3 | 1-3 | 1-3 | 1-3 | 1-3 | 1-3 | 1-3 | 1-3 | 1-3 |
| (nucleotide | | (9) | (9) | (9) | (9) | (9) | (9) | (9) | (9) | (9) |
| units) | 10 | 2-4 | 2-4 | 2-4 | 2-4 | 2-4 | 2-4 | 2-4 | 2-4 | 2-4 |
| | | (10) | (10) | (10) | (10) | (10) | (10) | (10) | (10) | (10) |
| | 11 | 2-4 | 2-4 | 2-4 | 2-4 | 2-4 | 2-4 | 2-4 | 2-4 | 2-4 |
| | | (11) | (11) | (11) | (11) | (11) | (11) | (11) | (11) | (11) |
| | 12 | 2-4 | 2-4 | 2-4 | 2-4 | 2-4 | 2-4 | 2-4 | 2-4 | 2-4 |
| | | (12) | (12) | (12) | (12) | (12) | (12) | (12) | (12) | (12) |
| | 13 | 2-4 | 2-5 | 2-5 | 2-5 | 2-5 | 2-5 | 2-5 | 2-5 | 2-5 |
| | | (12) | (13) | (13) | (13) | (13) | (13) | (13) | (13) | (13) |
| | 14 | 2-4 | 2-5 | 2-5 | 2-5 | 2-5 | 2-5 | 2-5 | 2-5 | 2-5 |
| | | (12) | (13) | (14) | (14) | (14) | (14) | (14) | (14) | (14) |
| | 15 | 2-4 | 2-5 | 2-5 | 3-6 | 3-6 | 3-6 | 3-6 | 3-6 | 3-6 |
| | | (12) | (13) | (14) | (15) | (15) | (15) | (15) | (15) | (15) |
| | 16 | 2-4 | 2-5 | 2-5 | 3-6 | 3-6 | 3-6 | 3-6 | 3-6 | 3-6 |
| | | (12) | (13) | (14) | (15) | (16) | (16) | (16) | (16) | (16) |
| | 17 | 2-4 | 2-5 | 2-5 | 3-6 | 3-6 | 3-6 | 3-6 | 3-6 | 3-6 |
| | | (12) | (13) | (14) | (15) | (16) | (17) | (17) | (17) | (17) |
| | 18 | 2-4 | 2-5 | 2-5 | 3-6 | 3-6 | 3-6 | 3-7 | 3-7 | 3-7 |
| | | (12) | (13) | (14) | (15) | (16) | (17) | (18) | (18) | (18) |
| | 19 | 2-4 | 2-5 | 2-5 | 3-6 | 3-6 | 3-6 | 3-7 | 3-7 | 3-7 |
| | | (12) | (13) | (14) | (15) | (16) | (17) | (18) | (19) | (19) |
| | 20 | 2-4 | 2-5 | 2-5 | 3-6 | 3-6 | 3-6 | 3-7 | 3-7 | 4-8 |
| | | (12) | (13) | (14) | (15) | (16) | (17) | (18) | (19) | (20) |
| | 21-35 | 2-4 | 2-5 | 2-5 | 3-6 | 3-6 | 3-6 | 3-7 | 3-7 | 4-8 |
| | | (12) | (13) | (14) | (15) | (16) | (17) | (18) | (19) | (20) |

In general therefore a T-oligonucleotide will have 1-8, 2-7, 3-6, mismatches, e.g. 1-3, 2-4, 2-5, 3-6, 4-8 mismatches, with its corresponding FRET-oligonucleotide.

Where the maximum number of base pairs that could be formed in a pair is 9, (e.g. when the T-oligonucleotide is 9 nucleotide units in length and the FRET-oligonucleotide is at least 12 nucleotide units in length) there may be 1-3 mismatches.

Where the maximum number of base pairs that could be formed in a pair is 10, (e.g. when the T-oligonucleotide is 10 nucleotide units in length and the FRET-oligonucleotide is at least 12 nucleotide units in length) there may be 2-4 mismatches.

Where the maximum number of base pairs that could be formed in a pair is 11, (e.g. when the T-oligonucleotide is 11 nucleotide units in length and the FRET-oligonucleotide is at least 12 nucleotide units in length) there may be 2-4 mismatches.

Where the maximum number of base pairs that could be formed in a pair is 12, (e.g. when the T-oligonucleotide is 12 nucleotide units in length and the FRET-oligonucleotide is at least 12 nucleotide units in length, or when the T-oligonucleotide is 13-20 nucleotide units in length and the FRET-oligonucleotide is 12 nucleotide units in length) there may be 2-4 mismatches.

Where the maximum number of base pairs that could be formed in a pair is 13, (e.g. when the T-oligonucleotide is 13 nucleotide units in length and the FRET-oligonucleotide is at least 13 nucleotide units in length, or when the T-oligonucleotide is 14-20 nucleotide units in length and the FRET-oligonucleotide is 13 nucleotide units in length) there may be 2-5 mismatches.

Where the maximum number of base pairs that could be formed in a pair is 14, (e.g. when the T-oligonucleotide is 14 nucleotide units in length and the FRET-oligonucleotide is at least 14 nucleotide units in length, or when the T-oligonucleotide is 15-20 nucleotide units in length and the FRET-oligonucleotide is 14 nucleotide units in length) there may be 2-5 mismatches.

Where the maximum number of base pairs that could be formed in a pair is 15, (e.g. when the T-oligonucleotide is 15 nucleotide units in length and the FRET-oligonucleotide is at least 15 nucleotide units in length, or when the T-oligonucleotide is 16-20 nucleotide units in length and the FRET-oligonucleotide is 15 nucleotide units in length) there may be 3-6 mismatches.

Where the maximum number of base pairs that could be formed in a pair is 16, (e.g. when the T-oligonucleotide is 16 nucleotide units in length and the FRET-oligonucleotide is at least 16 nucleotide units in length, or when the T-oligonucleotide is 17-20 nucleotide units in length and the FRET-oligonucleotide is 16 nucleotide units in length) there may be 3-6 mismatches.

Where the maximum number of base pairs that could be formed in a pair is 17, (e.g. when the T-oligonucleotide is 17 nucleotide units in length and the FRET-oligonucleotide is at least 17 nucleotide units in length, or when the T-oligonucleotide is 18-20 nucleotide units in length and the FRET-oligonucleotide is 17 nucleotide units in length) there may be 3-6 mismatches.

Where the maximum number of base pairs that could be formed in a pair is 18, (e.g. when the T-oligonucleotide is 18 nucleotide units in length and the FRET-oligonucleotide is at least 18 nucleotide units in length, or when the T-oligonucleotide is 19-20 nucleotide units in length and the FRET-oligonucleotide is 18 nucleotide units in length) there may be 3-7 mismatches.

Where the maximum number of base pairs that could be formed in a pair is 19, (e.g. when the T-oligonucleotide is 19 nucleotide units in length and the FRET-oligonucleotide is at least 19 nucleotide units in length, or when the T-oligonucleotide is 20 nucleotide units in length and the FRET-oligonucleotide is 19 nucleotide units in length) there may be 3-7 mismatches.

Where the maximum number of base pairs that could be formed in a pair is 20, (e.g. when the T-oligonucleotide is 20 nucleotide units in length and the FRET-oligonucleotide is at least 20 nucleotide units in length) there may be 4-8 mismatches.

A mismatch may occur where a base pair does not form between the bases on the two strands. This may be because complementary bases are not present at the appropriate position in the T-oligonucleotide or the FRET-oligonucleotides. Non-complementary base pairs may comprise or consist of naturally occurring base (e.g. A, T, C, G, U). Examples of naturally occurring non-complementary base pairs include A-G, G-A, A-C, C-A, A-A, T-T, T-G, G-T, T-C, C-T, C-C, G-G, U-U, U-G, G-U, U-C, C-U pairs may be present. The presence of other bases may also give rise to mismatches (e.g. non-naturally occurring or modified bases).

The location (or relative location if there are multiple mismatches) also contributes to the stability of the oligonucleotide pair. The presence of two or more adjacent mismatches will tend to impact hybridisation to a greater degree than two mismatches that are not adjacent (e.g. separated by at least 1, 2, 3, 4, 5, 6, 7, 8, 9 complementary base pairs). T-oligonucleotides for use in accordance with the invention may therefore have a sequence that gives rise to two or more adjacent mismatches, e.g. 3 or more or 4 or more or 5 or more adjacent mismatches. Other T-oligonucleotides for use in accordance with the invention may therefore have a sequence that gives rise to two mismatches that are not adjacent (e.g. separated by at least 1, 2, 3, 4, 5, 6, 7, 8, 9 complementary base pairs), or at least three mismatches that are non-adjacent (e.g. every pair of mismatches is separated by at least 1, 2, 3, 4, 5, 6, 7, 8, 9 complementary base pairs). Further T-oligonucleotides for use in accordance with the invention may have a sequence that gives rise to a mixture of adjacent and non-adjacent mismatches.

The inclusion of G or C bases in mismatches toward the end of the T-oligonucleotide, e.g. within 5, 4, 3, 2 nucleotide units of the end of the T-oligonucleotide, further disrupts hybridisation. T-oligonucleotides for use in accordance with the invention may therefore have a sequence that gives rise to one or more mismatches located toward the end of the T-oligonucleotide, e.g. within 5, 4, 3, 2 nucleotide units of the end of the T-oligonucleotide. Preferably one of the mismatched bases in each mismatched pair is G or C or a functional equivalent thereof (e.g. G or C may be present in the T-oligonucleotide or the corresponding FRET-oligonucleotide at this position such at a mismatch is present).

The inclusion of mismatches that create flexible extrusions or generate new structural conformations further disrupts hybridisation. The introduction of mismatches may also generate mononucleotide repeats. Mononucleotide repeats are repeats of a single nucleotide, so for example one or more mismatches in which at least one nucleotide in the mismatch is part of a mononucleotide repeat (e.g. where the repeat is of at least 2, 3, 4, 5, 6 nucleotides) may be used. The introduction of mismatches may also generate secondary structures that may impact hybridisation between the FRET-oligonucleotide and T-oligonucleotide (e.g. the formation of hairpin structures in the T-oligonucleotide, e.g. of at least 2, 3, 4, 5 nucleotide units in length).

In view of the above, any individual T-oligonucleotide for use in accordance with the invention may have one or more of the above properties:
(i) 0-40%, 10-40% or 20-40% mismatches with its corresponding FRET-oligonucleotide, as compared to the maximum number of base pairs that could be formed in such a pair;
(ii) two or more adjacent mismatches (e.g. 3 or more or 4 or more or 5 or more adjacent mismatches);
(iii) two or more non-adjacent mismatches (e.g. separated by at least 1, 2, 3, 4, 5, 6, 7, 8, 9 complementary base pairs, or where there are more than two non-adjacent mismatches each pair of mismatches is separated by at least 1, 2, 3, 4, 5, 6, 7, 8, 9 complementary base pairs);
(iv) one or more mismatches located within 5, 4, 3, 2 nucleotide units of the end of the T-oligonucleotide, optionally wherein one of the mismatched bases in each mismatched pair is G or C or a functional equivalent thereof (e.g. G or C may be present in the T-oligonucleotide or the corresponding FRET-oligonucleotide at this position such at a mismatch is present);
(v) one or more mismatches that create flexible extrusions;
(vi) one or more mismatches that generate new structural conformations
(vii) one or more mismatches in which at least one nucleotide in the mismatch is part of a mononucleotide repeat (e.g. where the repeat is of at least 2, 3, 4, 5, 6 nucleotides);
(viii) one or more mismatches in which at least one nucleotide in the mismatches forms part of a secondary structure that may impact hybridisation between the FRET-oligonucleotide and T-oligonucleotide (e.g. the formation of hairpin structures in the T-oligonucleotide, e.g. of at least 2, 3, 4, 5 nucleotide units in length.

### Set of Binding Agent-T-Oligonucleotide Conjugates

A set of binding agent-T-oligonucleotide conjugates is used in methods according to the invention. In a set, a plurality of binding agent-T-oligonucleotide conjugates is used. Since the method is predicated on the ability to generate different fluorescent kinetic profiles, with each fluorescent kinetic profile being attributable to the presence of a specific T oligonucleotide, the selection of the different T-oligonucleotides that make up each set must be carried out in such a way as to generate multiple fluorescent kinetic profiles.

For each set of T-oligonucleotides there is a corresponding FRET oligonucleotide. The T-oligonucleotides in each set are selected so that different fluorescent kinetic profiles arise as a result of the association and dissociation of each T-oligonucleotide and its corresponding FRET oligonucleotide. Each set of binding agent T-oligonucleotide conjugates will contain two or more different T-oligonucleotides and the structural characteristics (e.g. sequence) of the T-oligonucleotide determines the fluorescent kinetic profile for its pair. A set will therefore comprise T-oligonucleotides that give rise to different fluorescent kinetic profiles as a result of association and dissociation with the corresponding FRET oligonucleotide.

For example, for a given FRET-oligonucleotide one member of the set of T-oligonucleotides may give rise to a pair that associates and dissociates relatively quickly, e.g. with a frequency of 15 blinking events per second, and another member of the set of T-oligonucleotides may give rise to a pair that associates and dissociates more slowly, e.g. with a frequency of 5 blinking events per second. In general oligonucleotide pairs with a greater number of mismatches will have a higher frequency of blinking events than pairs with a lower number of mismatches. In general, when comparing two pairs having fully complementary overlapping regions, the pair having a longer overlapping region will have a higher off time and a higher on time than a pair having a shorter overlapping region.

As referred to elsewhere herein. a set as defined herein may contain 2, 3, 4, 5, 6, 7, 8, 9, 10, 11, 12, 13, 14, 15, 16, 17, 18, 19, 20, 21, 22, 23, 24, 25 or at least 2, 3, 4, 5, 6, 7, 8, 9, 10, 11, 12, 13, 14, 15, 16, 17, 18, 19, 20, 21, 22, 23, 24, 25, or up to 2, 3, 4, 5, 6, 7, 8, 9, 10, 11, 12, 13, 14, 15, 16, 17, 18, 19, 20, 21, 22, 23, 24, 25 e.g. 1-25, 2-24, 3-23, 4-22, 5-21, 6-20, 7-19, 8-18, 9-17, 10-16, 11-15, 12-14, binding agent-T-oligonucleotide conjugates.

A T-oligonucleotide will in general hybridise only to its corresponding FRET-oligonucleotides, and not to other FRET-oligonucleotides or to other T-oligonucleotides.

Preferred sets of T-oligonucleotides and corresponding FRET-oligonucleotides include:
a set comprising (optionally consisting of):

one or both of the T-oligonucleotide(s) of
sequences
SEQ ID NO: 1
(5′-TTCCACATTACTTCT-3′)
and
SEQ ID NO: 2
(5′-ATCCCCATTACTTCT-3′),
and
the corresponding FRET-oligonucleotide of
sequence
SEQ ID NO: 7
(5′-AGAAGTAATGTGGAA-3′)

a set comprising (optionally consisting of):

at least one, of the T-oligonucleotide(s) of
sequences
SEQ ID NO: 3
(5′-GGGTGGCGTATGATAGCTAT-3′),
SEQ ID NO: 4
(5′-AGGTTAAGTATGATAGTTAT-3′),
SEQ ID NO: 5
(5′-GGGTTAAGCATGGTAGTTAT-3′)
and
SEQ ID NO: 6
(5′-AGGTTAAGCGTTATATTTAT-3′)
(for example at least 2, 3 or all 4 of the
recited T-oligonucleotides)
and
the corresponding FRET-oligonucleotide of
sequence
SEQ ID NO: 8
(5′-ATAACTATCATACTTAACCT-3′).

The two sets above may be used together as a group of sets, e.g. where the two FRET oligonucleotides contain emitter molecules which have overlapping emission spectrums, optionally which have the same emitter molecule.

Binding Agent and Target Molecule

The binding agent is any agent that can bind to a binding partner or target molecule of interest, e.g. which binds directly or indirectly to the target molecule of interest. The binding agent will in general be specific to the target molecule of interest, e.g. it may bind to the target molecule of interest with a higher affinity and/or specificity than to other components in the sample. The binding agent will in general bind to the target molecule of interest directly, e.g. via electrostatic bonds.

A target molecule may be any molecule or entity of interest for which detection is required. For example, a target molecule may include a peptide or protein, nucleic acid (RNA, DNA or any modification thereof), lipid, carbohydrate, small molecule. A target molecule may be present in or on a cell. Since the methods of the invention allow for multiple target molecules to be studied, it is not necessary for all target molecules to be of the same type. The method may therefore give rise to the observation of one or more of peptide or protein, nucleic acid (RNA, DNA or any modification thereof), lipid, carbohydrate, or small molecule or a combination of some or all of these.

The binding agent may be or may comprise a protein (e.g. a polypeptide) or may be or may comprise a non-protein molecule. Examples of a binding agent that is or comprises a protein include antibodies, antibody fragments, receptors (e.g. soluble receptors), ligands, lectins. Examples of a binding agent that is or comprises a non-protein molecule include carbohydrates and nucleic acid molecules or their derivatives, and aptamers.

The binding agent may be selected to have a high binding affinity for its target, for example a binding affinity with a dissociation constant in the micromolar or nanomolar range (e.g. lower than about 10⁻⁴ M, 10⁻⁵ M, 10⁻⁶ M, 10⁻⁷ M, 10⁻⁸ M or 10⁻⁹ M, or in the range of 10⁻⁴ M to 10⁻⁶ M, or 10⁻⁷ M to 10⁻⁹ M). Affinity is the strength of binding of a molecule (such as an antibody) to its ligand (such as an antigen). It is typically measured and reported by the equilibrium dissociation constant (KD). KD is the ratio of the antibody dissociation rate (“off rate” or Koff), how quickly it dissociates from its antigen, to the antibody association rate (“on rate” or Kon) of the antibody, how quickly it binds to its antigen. For example, an antibody with an affinity of ≤1 μM has a KD value that is 1 μM or lower (i.e., 1 μM or higher affinity) determined by a suitable in vitro binding assay. Suitable in vitro assays, such as Biolayer Interferometry (e.g., Octet) or surface plasmon resonance (e.g., Biacore System) can be used to assess affinities, as measured by KD values based on well-known methods. By selecting binding agents which have a high binding affinity for the target molecule, the selectivity and sensitivity of the method of the present invention may be increased. The term “antibody” is used to mean an immunoglobulin protein containing at least one V H and/or V L domain and that binds to a target molecule. Many antibody structures are known, e.g. full length antibodies including at least two heavy (H) chains and two light (L) chains inter-connected by disulfide bonds, heavy chain only antibodies such as camelid and shark HCAbs. Antibody fragments may include antigen binding portions of antibodies or thereof. Antibodies may be polyclonal or monoclonal; naturally occurring or synthetic, or modified forms thereof (e.g., humanized, chimeric).

The antigen-binding function of an antibody can be performed by fragments of a full-length antibody. As used herein, “antigen-binding portion” of an antibody, e.g. of a monoclonal antibody, refers to one or more fragments of an antibody that retain the ability to specifically bind to an antigen. Examples of binding fragments encompassed within the term “antigen-binding portion” of an antibody include (i) a Fab fragment (a monovalent fragment which contains the VH, VL, CL and CH1 domains); (ii) a F(ab′)2 fragment (a bivalent fragment comprising two Fab fragments linked by a disulfide bridge at the hinge region); (iii) a Fd fragment consisting of the VH and CH1 domains; (iv) a Fv fragment (VH and VL domains of one arm of an antibody (including scFv fragments, e.g. fragment in which a VH and VL portion are joined by a synthetic linker and multimers thereof)), (v) a dAb fragment (single VH domain); and (vi) an isolated complementarity determining region (CDR) or combination of at least two CDRs, which may e.g. be joined by a synthetic linker.

These antibody fragments are obtained using conventional techniques known to those with skill in the art, and the fragments are screened for utility in the same manner as are intact antibodies.

Example of binding agents that are or comprise non-protein molecules include binding agents comprising oligonucleotides (which may comprise deoxyribonucleotides, ribonucleotides and/or synthetic nucleotide residues, e.g. which may be a DNA, RNA or synthetic oligonucleotide).

### Conjugation of Binding Agent and the T-Oligonucleotide

The binding agent and the T-oligonucleotide are conjugated. By this it is meant that these two components of the conjugate are linked in a covalent or non-covalent manner This can be achieved by any suitable means. The components of the conjugate may be linked directly, or indirectly, e.g. via a linker.

In embodiments in which the binding agent is a nucleic acid or an aptamer, the coupling may be via a covalent phosphodiester bond, e.g. between the 5′ phosphate group of one nucleotide or functional equivalent thereof and the 3′OH group of another nucleotide or functional equivalent thereof. In such embodiments the binding agent component and the T-oligonucleotide component may be congruous or may be separated by one or more oligonucleotide units. In such a case the oligonucleotide units that do not participate in binding with the binding target or with the FRET-oligonucleotide may be described as linking oligonucleotide units. There may be e.g. 2-30, 3-25, 4-20, 5-15, 6-10 linking oligonucleotide units, or up to 2, 3, 4, 5, 6, 7, 8, 9, 10 linking oligonucleotide units.

In embodiments in which the binding agent is a protein, e.g. an antibody, the conjugation between the binding agent and the T-oligonucleotide may be via any appropriate covalent or non-covalent linkage. Suitable means known in the art include chemical crosslinking of the two components of the conjugate, e.g. via a linker, e.g. a crosslinker. Crosslinking reagents contain reactive ends to specific functional groups (primary amines, sulfhydryls, etc.) on proteins or other molecules. The availability of several chemical groups in proteins and peptides make them targets for conjugation using crosslinking methods. Crosslinkers contain at least two reactive groups, which target common functional groups found in biomolecules such as proteins and nucleic acids. The functional groups that are commonly targeted for bioconjugation include primary amines, sulfhydryls, carbonyls, carbohydrates and carboxylic acids. Other examples include click chemistry using modified T-oligos (e.g. with maleimide, NHS or azide groups). The two reactive groups of a crosslinker are separated by a spacer, which may e.g. comprise PEG. The spacer is in general 3 to 50 angstroms in length,

The T-oligo is preferably conjugated at a location on the binding agent that does not interfere with the ability of the binding partner to bind to its binding partner. For example, for binding agent that is an antibody or antibody fragment, the T-oligo is preferably conjugated at a constant region, and/or in a region that does not interfere with antibody binding to its target, e.g. away from the antigen binding site.

Each molecule of binding agent may be conjugated to a single T-oligonucleotide molecule, or to multiple T-oligonucleotide molecules. Where multiple T-oligonucleotide molecules are attached per molecule of binding agent, each of those T-oligonucleotide molecules has the same oligonucleotide sequence (since the sequence of the T-oligonucleotide is unique to the binding agent to which it is conjugated).

By way of example 1-10, 2-9, 3-8, 4-7, or 5-6 T-oligonucleotide molecules are conjugated to each molecule of binding agent. Each T-oligonucleotide molecule may be attached at a different location on the binding agent. The number of T-oligonucleotide molecules attached to each binding agent molecule will depend on the nature of the conjugation that is used. This number of molecules of T oligonucleotides per molecule of binding agent may be absolute, e.g. if each molecule of binding agent has the same number of molecules of T oligonucleotide. Alternatively, the number of molecules of T oligonucleotides per molecule of binding agent may represent an average number of T oligonucleotides per molecule of binding agent.

### FRET Oligo

The FRET-oligonucleotide is an oligonucleotide as defined elsewhere herein. The oligonucleotide has attached, incorporated, and/or conjugated to it a fluorescence emitter molecule (emitter molecule) and a quenching entity. The fluorescence emitter molecule may be a Forster Resonance Energy Transfer (FRET) donor, and the quenching entity may be a corresponding FRET acceptor; upon illumination at a wavelength to cause excitation of the emitter molecule, no fluorescence is observed at the emitter molecule's emission wavelength due to efficient energy transfer to the quenching entity when the FRET-oligonucleotide is in solution. However, upon FRET-oligonucleotide hybridisation with a T oligonucleotide, the emitter and quenching entity are separated physically and the rate of energy transfer between the emitter and the quencher is e.g. FRET is reduced, so that fluorescence may be observed at the emitter molecule's emission wavelength.

The emitter molecule and the quenching entity are thus located on the oligonucleotide at positions such that when the FRET-oligonucleotide is fully linear (e.g. when it is hybridised to a T-oligonucleotide or an oligonucleotide that is fully complementary in sequence to the FRET oligonucleotide) the emitter molecule and quenching entity are spatially separated such that the transfer of energy between the two molecules does not occur and fluorescence may be observed at the emitter molecule's emission wavelength.

In contrast, when the FRET-oligonucleotide is in solution its configuration is disordered and the fluorescence emitter molecule and quenching entity are not spatially separated. As such, transfer of energy between the emitter molecule and quenching entity does not occur and fluorescence may not be or is not observed at the emitter molecule's emission wavelength. Depending on the nature of the quenching entity the result of that energy transfer may be that all fluorescence is quenched or suppressed (e.g. if a dark quencher or a black hole quencher is used, since a dark quencher in general absorbs excitation energy from an emitter molecule and dissipates the energy as heat, i.e. no fluorescence is detected at any). Alternatively, the quencher molecule itself may emit fluorescence, but this is emitted from the quencher molecule at a wavelength that is distinct from the emitter molecule's emission wavelength. This means that irrespective of the nature of the quencher or the mechanism of quenching, quenching of the emitter molecule's emission results in a reduction of or an absence of fluorescence emission at the emitter molecule's emission wavelength. A reduction may be a reduction to less than 60, 50, 40, 30, 20, 10% of the fluorescence emission that is observed at the emitter molecule's emission wavelength in the absence of the quencher entity.

In view of the above, fluorescent emission is in general terms detectable from the fluorescence emitter molecule in the FRET-oligonucleotide at the emitter molecule's emission wavelength when the FRET-oligonucleotide is hybridised to a T-oligonucleotide but not (or at a reduced level) when the FRET-oligonucleotide is free in solution. The association between the FRET-oligonucleotide and T-oligonucleotide turns the fluorescence emission at the emitter molecule's emission wavelength “on” and the dissociation turns the fluorescence emission at the emitter molecule's emission wavelength “off”. The FRET-oligonucleotide and T-oligonucleotide are designed to hybridise imperfectly so that they readily dissociate and reassociate, giving rise to a distinct fluorescent kinetic profile that is characteristic of each pair. The fluorescent kinetic profile is made up of both the frequency of blinking and the duration of the “on” signal, as discussed in more detail below.

The fluorescence emitter molecule and a quenching entity are therefore attached, incorporated, and/or conjugated to the oligonucleotide at a distance that precludes energy transfer (e.g. FRET) between the fluorescence emitter molecule and the quenching entity when the oligonucleotide is in the bound conformation. Alternatively stated the distance between the two components in the oligonucleotide exceeds the Forster Radius of the FRET-oligonucleotide emitter molecule. Expressed in terms of nucleotide units the emitter molecule and a quenching entity are therefore attached, incorporated, and/or conjugated to the oligonucleotide at a distance of at least 12 nucleotide units in length. The distance corresponds to a distance of at least 5, 6, or 7 nm for most FRET pairs, preferably at least 9 nm for most FRET pairs. These distances are measured or expressed in terms of the FRET-oligonucleotide in its linear, fully extended form, or in the bound conformation.

For example for a FRET oligo using Atto425/Atto488 the Forster radius is 5.2 nm. For a FRET oligo using Atto488/Atto550, the Forster radius is 6.3 nm. For a FRET oligo using Atto550/Atto647 the Forster radius is 6.5 nm. For a FRET-oligo with Atto647/BHQ3 (dark quencher, the Forster radius is 7.0 nm).

This may be achieved by locating the fluorescence emitter molecule and a quenching entity at positions within the FRET-oligonucleotide that are separated by at least 12 nucleotide units. For example if the FRET-oligonucleotide is 12 oligonucleotides in length or longer the two molecules may be located so that the fluorescence emitter molecule is located on the 5′ most oligonucleotide unit of the FRET-oligonucleotide and the quenching entity is located on the 3′ most oligonucleotide unit of the FRET-oligonucleotide or vice versa. Alternatively the two molecules may be located on a FRET-oligonucleotide such that they are located on nucleotide units that are at least 11 oligonucleotide units apart (e.g. at least 11, 12 13, 14, 15, 16, 17, 18, 19, 20 oligonucleotide units apart). For example two molecules are 11 oligonucleotide units apart when they are on nucleotide unit no 1 and no 12, 1 and 13, 1 and 14. 1 and 15, 1 and 16, 1 and 17, 1 and 18, 1 and 19, 1 and 20, 2 and 13, 2 and 14, 2 and 15, 2 and 16 etc.).

In certain embodiments at least one of and preferably both of the fluorescence emitter molecule and the quenching entity are located on a terminal oligonucleotide units. For example the fluorescence emitter molecule may be located on the 5′ most oligonucleotide unit of the FRET-oligonucleotide and the quenching entity may be located on the 3′ most oligonucleotide unit of the FRET-oligonucleotide, or vice versa.

A FRET-oligonucleotide preferably has a sequence that does not form secondary structure, such as hairpins or hairpin loops. This is because it would be energetically favourable for a FRET-oligonucleotide having such secondary structure to form this secondary structure in solution. The existence of the secondary structure would decrease the rate at which dimers could be formed with the T-oligonucleotide. A FRET-oligo preferably has a sequence that does not allow self-ligation, or cross-dimerisation with other FRET-oligonucleotides that may be used in the methods of the invention. Self-ligation, or cross-dimerisation with other FRET-oligonucleotides that may be used in the methods of the invention would generate fluorescence in the absence of hybridisation to a T-oligonucleotide. A FRET-oligonucleotide should also not be complementary to a DNA sequence present in the sample.

The fluorescence emitter molecule and a quenching entity are in certain embodiments located on the 5′ most and 3′ most oligonucleotide unit of the FRET-oligonucleotide. The fluorescence emitter molecule and a quenching entity may be located so that the fluorescence emitter molecule is located on the 5′ most oligonucleotide unit of the FRET-oligonucleotide and the quenching entity is located on the 3′ most oligonucleotide unit of the FRET-oligonucleotide or vice versa.

### Fluorescence Emitter Molecules and Quenching Entities

A fluorescent emitter molecule is a molecule that generates a predetermined fluorescent wavelength emission on illumination with an appropriate spectral emission.

A quenching entity as referred to herein is a molecule to which the predetermined fluorescent wavelength emission emitted by the fluorescent emitter molecule is transferred, e.g. by Förster resonance energy transfer (FRET). When a quencher is present, e.g. in an appropriate location, the excited fluorescent emitter molecule can return to the ground state by transferring its energy to the quencher, without the emission of light, while the quencher is promoted to its excited state. Energy is transferred from the emitter (or donor) molecule while the emitter is in the excited state. FRET is based on classical dipole-dipole interactions between the transition dipoles of the donor and acceptor and is extremely dependent on the donor-acceptor distance, R, falling off at a rate of 1/R 6.

When a fluorescent emitter molecule is excited with illumination with an appropriate spectral emission (e.g. at a particular wavelength), it is promoted to an excited state. In the absence of a quencher, the excited molecule emits light at its emission wavelength in returning to the ground state. In the presence of a quencher at the appropriate location, transfer of the energy to the quencher results in a reduction in or an absence of fluorescence emission at the emitter molecule's emission wavelength. Depending on the nature of the quencher, the result of the energy transfer may be that no fluorescence is emitted (e.g. if a dark quencher or black hole quencher is used). Alternatively a quencher that gives rise to emission of light at a different wavelength to the emission wavelength may be used, in which case there will still be a reduction in or an absence of fluorescence emission at the emitter molecule's emission wavelength.

For there to be energy transfer between the two molecules (e.g. by FRET) there must be an appropriate donor-acceptor spectral overlap; the emission spectrum of the emitter molecule must overlap with the absorption spectrum of the quencher entity. So only certain pairs of fluorescence emitter molecules and quenching entities can be used. Optimal fret pairs can be designed at any wavelength so long as the emission spectrum of the donor overlaps with the excitation spectrum of the acceptor. The optimal excitation wavelength of the illumination source should be based on the fluorescence emission of the FRET-oligonucleotides during hybridisation.

Appropriate pairs of fluorescence emitter molecules and quenching entities can be selected. Examples of suitable pairs include Atto425/Atto488 (e.g. excite with a uv illumination source and detect blinking at the Atto425 emission wavelength), Atto488/Atto550 (e.g. excite with a blue illumination source and detect blinking at the atto488 emission wavelength), Atto550/Atto647 (e.g. excite with a green illumination source and detect blinking at the Atto550 emission wavelength), Atto647/BHQ3 (dark quencher) (excite with a red illumination source and detect blinking at the Atto647 emission wavelength). Cy5 fluorophore and a BHQ-3 dark quencher (e.g. excite with a 639 nm laser and detect blinking at the Cy 5 emission wavelength).

BHQ-1, BHQ-2 and BHQ-3 are widely used Black Hole quenchers that are well known in the art, see for example https://www sigmaaldrich.com/technical-documents/articles/biology/black-hole-quenchers.html. Other dark quenchers include Dabsyl (dimethylaminoazobenzenesulfonic acid) which absorbs in the green spectrum and is often used with fluorescein, Qxl quenchers (which span the full visible spectrum), Iowa black FQ (which absorbs in the green-yellow part of the spectrum.), Iowa black RQ (which blocks in the orange-red part of the spectrum), IRDye QC-1 (which quenches dyes from the visible to the near-infrared range (500-900 nm)).

Other examples of pairs of FRET probes are set out in Yoshiyuki Arai and Takeharu Nagai (2013) Extensive use of FRET in biological imaging, Microscopy 0(0): 1-10 (2013), for example the following emitter molecules and quenching entities are suitable examples:

| Emitter molecule | Quencher entity |
|---|---|
| Sirius | mseCFP |
| BFP | GFP (C-S65T) |
| T-Sapphire | mOrange |
| mAmetrine | tdTomato |
| T-Sapphire | PSmOrange2 |
| mCellurian | mCitrine |
| mTurquoise | eYFP(cpVenus) |
| eCFP | tdTomato |
| eCFP | mDsRed |
| eCFP | YPet |
| CyPet | YPet |
| LSSmOrange | LSSmKate2 |
| CFP | YFP |
| mseCFP | PA-GFP |
| mTFP1 | mCitrine |
| GFP | TMR 470 |
| GFP | Alexa546 |
| Alexa488 | Alexa594 |
| Alexa488 | TMR |
| eGFP | mDsRed |
| eGFP | mRFP |
| SYFP | mRFP |
| TagGFP | TagRFP |
| Clover | mRuby2 |
| Cy3 | Atto647N |
| TMR | Atto647 |
| TMR | Cy5 |
| TMR | IC5 |
| Cy3 | Cy5 |
| mKO | mCherry |
| tagRFP | mPlum |
| mOrange2 | mKate2 |
| mOrange | mCherry |
| Qdot510, Qdot530, Qdot555, QDot525 | Cy3 |
| Cybate | Cypate |
| MMPSense 750 | FAST MMPSense |
| mTFP1 | eYFP 770 |
| eGFP | mStrawberry |
| RLuc, RLuc8 | EYFP |
| RLucX | Venus |

Reference is made herein to an emitter molecule's emission wavelength. It is well known that emission from an emitter molecule can be represented as a spectrum, with emission occurring at multiple wavelengths around a peak wavelength, or emission maximum. The precise shape of this spectrum as well as the emission maximum can be determined readily for each emitter molecule. Where reference is made to an emitter molecule's emission wavelength it is generally meant one or more wavelengths, which are at or close to the emitter molecule's emission maximum (e.g. the emission maximum of the emitter molecule, or a window of up to 1 nm, up to 2 nm, up to 3 nm, up to 4 nm, up to 5 nm, up to 10 nm, up to 15 nm up to 20 nm, up to 25 nm, up to 30 nm, up to 40 nm, up to 50 nm on either side of the emission maximum of any given emitter molecule).

In practical terms, for any given emitter molecule its fluorescence will be observed in a channel, which is a predefined window of wavelengths which is selected based on the emission spectrum of the emitter molecule. The channel may be e.g. up to 1 nm, up to 2 nm, up to 3 nm, up to 4 nm, up to 5 nm, up to 10 nm, up to 15 nm up to 20 nm, up to 25 nm, up to 30 nm, up to 40 nm, up to 50 nm on either side of the emission maximum of any given emitter molecule. So when reference is made e.g. to observing or detecting or to the presence or to the absence or a reduction in the kinetic fluorescent profile in an emitter's emission wavelength, this may refer to the fluorescence at the emitter molecule's emission maximum or to the fluorescence in a predetermined window that surrounds the emitter molecule's emission maximum.

The skilled person is able to select one or more appropriate channels for observing and emission of any given emitter molecule or molecules. The size of the channel can be determined readily and adapted to the particular experimental parameters. In general where a given set up is used in which a single channel is to be used to observe fluorescence emission, this will be a bigger window than the window for an experimental set up in which multiple channels are being used. The skilled person also knows to use appropriate parameters to avoid overlap of absorption and emission spectra, for example. Any appropriate channel may be selected using routine methods to observe the required fluorescence kinetic profile, depending on the nature of the one or more emitter molecules that are being used. For example where a single emitter molecule is being used, a single channel may be used and in this case the window can be relatively broad (e.g. up to 30 nm 40 nm, 50, 50 nm, 70 nm, 80 nm, 90 nm, 100 nm). If fluorescent kinetic profiles from multiple emitter molecules are being used each window would in general be narrower, e.g in order to avoid overlap between the various spectra, e.g. up to 1 nm, up to 2 nm, up to 3 nm, up to 4 nm, up to 5 nm, up to 10 nm, up to 15 nm up to 20 nm, up to 25 nm, up to 30 nm. The fluorescent kinetic profile may be observed or detected at any single or group of wavelengths within the excitation spectrum of the emitter molecule.

### Illumination

The method includes the step of illumination in which the fluorescent emitter molecules are illuminated using an appropriate spectral emission, thereby causing the fluorescent emitter molecules to generate its predetermined fluorescent wavelength emission. This may also be referred to as “excitation” and an appropriate wavelength may be selected for this purpose based on the known properties of the fluorescent emitter molecule or combinations thereof that are being used. A single source may be used to excite multiple fluorescent emitter molecules, if used. Alternatively a distinct source may be used for each fluorescent emitter molecule that is to be used, or a combination thereof may be used. Fluorescent emitter molecules have well characterised excitation or absorption spectra and selecting a suitable wavelength or combinations thereof for excitation of all of the fluorescent emitter molecules will be a matter of routine.

### Transient Hybridisation and the Fluorescent Kinetic Profile

As discussed above the T-oligonucleotide and FRET-oligonucleotide pairs are not fully complementary and hybridise transiently, i.e. there is with dissociation and reassociation between the two components of the pair. This dissociation and reassociation between the pair generates a fluorescent kinetic profile. As a result of the sequence differences between the T-oligonucleotides in the set, the fluorescent kinetic profile is unique within that set to that pair.

There are multiple components of the fluorescent kinetic profile. Dissociation of the pair turns the fluorescent signal in the FRET-oligonucleotide emitter molecule's emission wavelength “off” and reassociation between the pair turns the signal in the FRET-oligonucleotide emitter molecule's emission wavelength “on”, because only in the hybridised state are the fluorescence emitter molecule and a quenching entity sufficiently separated to prevent quenching of the emission in the FRET-oligonucleotide emitter molecule's emission wavelength. The fluorescent kinetic profile may be characterised in a number of different ways. Any number of numerical metrics or features including spatio-temporal information may be extracted from fluorescent kinetic profiles. Non-limiting examples of these metrics include (i) the average period of time between each fluorescence emission (also referred to as the off-time), (ii) the average duration of the fluorescence emission (also referred to as the on-time), and (iii) the rate of occurrences of fluorescence emission. At least one, at least 2 or at least 3 such metrics can be extracted from a fluorescent kinetic profile and used to describe it. Metrics other than these exemplary metrics may also be used.

Moreover, the turning off and on of a fluorescence emission, conceptualised as “blinking”, can be used to define numerical metrics that describe the hybridisation affinity between each T-oligonucleotide and the corresponding FRET-oligonucleotide. Any number of metrics can be derived from blinking kinetics, including at least (i) the Kon (Association rate constant, expressed in M-1.s-1) and (ii) the Koff (Dissociation rate constant, expressed in s-1). As each T-oligonucleotide has been engineered to exhibit unique hybridisation, and ergo blinking, kinetic profiles, any individual or combination of metrics may be used to distinguish the identity of a T-oligonucleotide under observation. These affinity metrics can also be a way of describing the fluorescent kinetic profile.

The number of occurrences of fluorescence emission in the FRET-oligonucleotide emitter molecule's emission wavelength in a given time frame means the number of times that the fluorescence emission is switched on in the FRET-oligonucleotide emitter molecule's emission wavelength in a given time frame. The transient nature of the hybridisation between the two components of the pair means that the fluorescence emission in the FRET-oligonucleotide emitter molecule's emission wavelength will appear and disappear with time, with the emission appearing in the FRET-oligonucleotide emitter molecule's emission wavelength when the pair is in hybridised form. The number of occurrences of fluorescence emission, or the number of peaks of fluorescence intensity can be expressed as a number per time unit, e.g. per S. Assuming a standard concentration of FRET-oligonucleotide, higher rate of blinking typically indicates a low-affinity hybridisation between the FRET-oligonucleotide and the T-oligonucleotide, e.g. when a T-oligonucleotide contains a high number of mismatches. Conversely, assuming a standard concentration of FRET-oligonucleotide, a low number of blinks indicates a high-affinity hybridisation.

The various metrics, including the non-limiting examples (i) the average period of time between each fluorescence emission (also referred to as the off-time), (ii) the average duration of the fluorescence emission (also referred to as the on-time), and (iii) the rate of occurrences of fluorescence emission allow the profile to be described in different ways. The on-time is the duration of the fluorescence emission, and can also be described as the bound state, or the bright time. This parameter is intrinsic to the nature of the hybridisation between the FRET- and T-oligonucleotides. This is because the ability of the two components of the pair to remain hybridised once they have formed a pair depends on the sequences of the overlapping regions. In contrast the off-time, the duration of the absence of fluorescence emission (which can also be described as the unbound state/dark time) depends on the sequences of the overlapping regions, but also depends on the concentration of the FRET-oligonucleotide that is added to immobilised T-oligonucleotides. This is because the likelihood of two members of a pair hybridising depends not only on the strength of their interaction but also on the chances of the two members encountering each other.

As noted above, the kinetics of the FRET- and T-oligonucleotides can also be described in terms of their dissociation (unbinding) and association (binding) rate constants, K_{off} and Kₒₙ, respectively. In that case, the Kₒₙ is influenced by the FRET-oligo concentration and its sequence (e.g. the presence of a hairpin will lower the Kₒₙ) while the K_{off} is influenced by the stability of the hybridisation. So the longer the overlapping region for instance, the smaller the K_{off} (the unbinding occurs slowly); the introduction of mismatches destabilises the hybridisation and therefore increases the K_{off} (the dissociation occurs rapidly).

Certain of the metrics that can be used to describe a fluorescent kinetic profile, such as the number of occurrences (peaks of fluorescence intensity) within a designated time frame, on time and off time for each oligonucleotide pair at a given concentration of T-oligonucleotides and FRET-oligonucleotide can be measured and/or calculated experimentally, by immobilising the T-oligonucleotides and contacting the immobilised T-oligonucleotides with a FRET-oligonucleotide and observing the fluorescence kinetic profile for each immobilised T-oligonucleotide. Information about at least one metric for each given pair, forms part of the process of assigning an identity to one or more pixels in the observed sample. The assignment can be based on at least one metric of the fluorescent kinetic profile of the sample at that pixel that is observed and/or calculated.

It is possible to carry out this deconvolution on the basis of one metric. In certain embodiments at least one, at least two at least three, at least four metrics of the fluorescent kinetic profile are used. In general, the method can be rendered more efficient by using multiple metrics, however as long as the metrics used are distinguishable (e.g. in that an identity can be assigned to the T oligonucleotide on the basis of that metric) it can be used alone. In certain embodiments it may be necessary to rely on more than one metric, and the metrics in combination may be distinguishable from the combination of metrics for another given fluorescent kinetic profile.

Certain components of the fluorescent kinetic profile can also be explored, e.g. during the process of designing oligonucleotide sets, by determining fluorogenic profiles in solution, e.g. as shown in Example 2, where increasing concentrations of T-oligonucleotides are incubated with a FRET-oligonucleotide. In solution, the hybridisation of the FRET-oligonucleotide to each T-oligonucleotide generates distinct fluorogenic profiles with the more disruptive mismatches leading to a seemingly reduced fluorogenicity.

In practice, when the methods of the invention are carried out the T-oligonucleotides are immobilised on the sample by virtue of the binding agent with which the T-oligonucleotide is conjugated. The fact that the binding agent-T-oligonucleotide conjugates are immobilised means that the fluorescent kinetic profile at any one or more pixels can be observed. It is therefore possible to observe the fluorescent kinetic profile in the FRET-oligonucleotide emitter molecule's emission wavelength of a particular FRET-oligonucleotide T-oligonucleotide pair in advance of using such a pair in the methods of the invention by immobilising T-oligonucleotides and adding FRET-oligonucleotides to the immobilised T-oligonucleotides. In this way the fluorescent kinetic profile of each pair can be observed and it can be verified that a set of T-oligonucleotides generates appropriate fluorescent kinetic profiles to be useful in the methods of the invention, and in particular that a fluorescent kinetic profile that is unique within that set to that pair is present. Further the nature of the profile (including for example observing or calculating one or more metric of the fluorescent kinetic profile) for each pair can be determined.

### Observation

Standard fluorescent microscopy optical systems may be used with this technique, which is compatible with standard microscope bases and illumination systems. In order to observe the fluorescence profile emanating from each pixel in an observed sample, image acquisition must be performed over time. For this task, a high-frame rate (>100 frames per second) Complementary Metal-Oxide-Semiconductor (CMOS) camera may be used; since the high frame rate allows for increased accuracy in profiling of fluorescent kinetic profiles, although any type of imaging device capable of detecting single molecule would be suitable.

For each sample images may be observed and/or recorded over time, e.g. with a total acquisition time of up to 1, 2, 3, 4, 5, 10, 20, 30, 40, 50, 60 seconds, e.g. 10-120, 20-110, 30-100, 40-80, 50-70 seconds, preferably 0-2, 2-5, 2-10 seconds. Time traces of fluorescence intensity can be generated to show the kinetic profile of specific regions of interest in the image stack.

The image acquisition is in general performed in a channel. As used herein a channel refers to a predefined window of wavelengths including the FRET-oligonucleotide emitter molecule's emission wavelength or wavelengths and which is selected based on the emission spectrum of the emitter molecule. The channel may be e.g. up to 1 nm, up to 2 nm, up to 3 nm, up to 4 nm, up to 5 nm, up to 10 nm, up to 15 nm up to 20 nm, up to 25 nm, up to 30 nm, up to 40 nm, up to 50 nm on either side of the emission maximum of any given emitter molecule. As also noted above, the size of the channel will depend on the emitter molecule or combination of emitter molecules that are being used. When reference is made e.g. to observing or detecting or to the presence or to the absence or a reduction in the kinetic fluorescent profile in an emitter's emission wavelength, this may refer to the fluorescence at the emitter molecule's emission maximum or to the fluorescence in a predetermined window that surrounds the emitter molecule's emission maxim. For example when the emitter molecule that is used is Atto550 the emission channel may represent the spectral bandwidth for red emission, when the emitter molecule that is used is Atto647 the emission channel may represent the spectral bandwidth for near infra-red emission, when the emitter molecule that is used is Atto488 the emission channel may correspond to the spectral bandwidth for green emission.

In addition the fluorescent kinetic profile may be recorded, e.g. for future analysis. This may be carried out using any appropriate techniques that are known in the art. In addition the fluorescent kinetic profile may be analysed. For example, at least one metric as discussed elsewhere may be derived from or calculated from the observed and/or recorded fluorescent kinetic profile.

### Image Processing

A longitudinal series of images is the set of consecutive, ordered images acquired. For each foci, e.g. in a longitudinal series of images, a fluorescent kinetic profile is observed. This may then be analysed, e.g. to derive or calculate at least one metric as discussed elsewhere. Any metric, e.g. any distinguishable metric of the fluorescent kinetic profile (including but not limited to one or more of the average number of occurrences (peaks of fluorescence intensity) within a designated time frame, the average on time and average off time can be measured or determined) can be used to assign an identity to one or more pixels in the observed sample based on the metric calculated from the fluorescent kinetic profile of the sample at that pixel.

A foci is the region in which the blinking emanating from a single molecule is observed. Typically it is about 3-5 pixels in diameter, however this may vary depending on the magnification and optics being used.

For pixels or foci where no binding agent-T-oligonucleotide conjugate has been immobilised (i.e. background pixels), stochastic fluorescence may be observed but the fluorescent kinetic profile for this background “noise” is highly distinguishable from targeted FRET-oligonucleotide T-oligonucleotides fluorescent kinetic profile, as shown for example in Example 2. Examples of these representative basic profiles for three distinct pairs are demonstrated in FIG. 4A (bottom profile) and FIG. 5A (bottom left profile). FIG. 13 also contains examples of representative profiles.

The fluorescent kinetic profile at each pixel (e.g. as defined with respect to at least one metric thereof) can be assigned an identity (e.g. to the specific T-oligonucleotide that has generated it) since each different FRET-oligonucleotide T-oligonucleotide pair generates a fluorescent kinetic profile that is unique within that set to that pair. In this way an identity is assigned to the pixel based on the fluorescent kinetic profile (e.g. as defined with respect to at least one metric thereof) of the sample at that pixel. This is shown, for example in FIG. 5A-FIG. 5D. A cell sample which has been contacted with antibodies recognising distinct molecular epitopes, and where each antibody species is conjugated to a distinct T-oligo has been contacted with a FRET-oligo corresponding to the conjugated T-oligos. Longitudinal image acquisition allows each pixel to be measured for fluorescence, and a profile to be observed or created, as shown in FIG. 5B. As each FRET-/T-oligo pair has a unique kinetic binding profile, this profile (e.g. as defined with respect to at least one metric thereof) can be used to link a pixel's fluorescence profile to its T-oligo identity, therefore cellular target. (FIG. 5C) Each pixel is assigned to a particular T-oligo identity, or to background and in (FIG. 5D) from this assignment, separate channel images for each imaging modality can be generated, despite the experiment using a single laser excitation wavelength. This means that it is possible to assign an identity to one or more pixels in the observed sample based on the fluorescent kinetic profile of the sample (e.g. as defined with respect to at least one metric thereof) at that pixel.

The fluorescent kinetic profiles (e.g. as defined with respect to at least one metric thereof) of the various pairs of FRET-oligonucleotide T-oligonucleotides can be determined experimentally e.g. as described elsewhere herein, so that when any fluorescent kinetic profile is observed in the sample, it is possible to compare the observed fluorescent kinetic profiles (e.g. as defined with respect to at least one metric thereof) with the known or predetermined fluorescent kinetic profiles of the various pairs (e.g. as defined with respect to at least one metric thereof) to determine which pair is present at each location (and hence which T-oligonucleotide is present in the pair and hence which binding agent has bound at that location).

This comparison and assignment can be performed by any suitable means. For example at least one metric of the fluorescent kinetic profile is calculated from the fluorescent kinetic profile. This metric or metrics may be compared to known or predetermined metric(s) of the fluorescent kinetic profile for each of the pairs of FRET-oligonucleotide T-oligonucleotides that has been used. This comparison allows an identity to be assigned to one or more pixels in the observed sample based on the metric calculated from the fluorescent kinetic profile of the sample at that pixel.

The identity of the pixels may then be used to generate one or more images of the sample, e.g. in which the location within the sample of one or more binding agents and/or target molecules is shown. Pixels assigned to each T-oligonucleotide identity can be separated into individual grayscale channels. Individual gray scale channel images can be composed into a pseudo-colored micrograph image, in which each T-oligonucleotide channel is shown as a different color.

Multiplexing with Groups of Sets

In certain embodiments step a comprises contacting the sample with a group of sets of binding agent-T-oligonucleotide conjugates, said group of sets comprising a first set of binding agent-T-oligonucleotide conjugates and one or more subsequent sets of binding agent-T-oligonucleotide conjugates; and step b comprises contacting the sample and any bound binding agents resulting from step a with a first FRET-oligonucleotide and one or more subsequent FRET-oligonucleotides, wherein said first FRET-oligonucleotide can hybridise to multiple T-oligonucleotides in the first set, to form multiple pairs, and wherein said each subsequent FRET-oligonucleotide can hybridise to multiple T-oligonucleotides in its corresponding set, to form multiple pairs, and wherein the dissociation and reassociation between each different pair generates a fluorescent kinetic profile that is (i) unique within each set to that pair, and (ii) unique within the group of sets to that pair.

In embodiments such as this a group of sets comprising a first set of binding agent-T-oligonucleotide conjugates and one or more subsequent sets of binding agent-T-oligonucleotide conjugates is used. A FRET oligonucleotide for each set of binding agent-T-oligonucleotide conjugates is therefore used. As discussed above, the use of multiple FRET oligonucleotides means that a wider range of fluorescent kinetic profiles can be generated.

A group of sets may contain 2, 3, 4, 5, 6, 7, 8, 9, 10, 11, 12, 13, 14, 15, 16, 17, 18, 19, 20, 21, 22, 23, 24, 25 or at least 2, 3, 4, 5, 6, 7, 8, 9, 10, 11, 12, 13, 14, 15, 16, 17, 18, 19, 20, 21, 22, 23, 24, 25, or up to 2, 3, 4, 5, 6, 7, 8, 9, 10, 11, 12, 13, 14, 15, 16, 17, 18, 19, 20, 21, 22, 23, 24, 25 e.g. 1-25, 2-24, 3-23, 4-22, 5-21, 6-20, 7-19, 8-18, 9-17, 10-16, 11-15, 12-14, sets of binding agent-T-oligonucleotide conjugates.

When groups of sets of binding agent-T-oligonucleotide conjugates are used, each corresponding FRET oligonucleotide may contain emitter molecules which have an emission spectrum that is overlapping (such that the emitted fluorescence can be detected in a single channel, e.g. they may contain emitter molecules which emit fluorescence of the same wavelength, or they may contain the same emitter molecule). This can be advantageous since it allows the observation step to be carried out for all oligonucleotide pairs simultaneously. By way of example when groups of set of binding agent-T-oligonucleotide conjugates are used, each different FRET oligonucleotide may contain an emitter molecule that is Atto550. In an alternative embodiment each different FRET oligonucleotide may contain an emitter molecule that is Atto647. In a further alternative embodiment each different FRET oligonucleotide may contain an emitter molecule that is Atto488.

Alternatively, when groups of sets of binding agent-T-oligonucleotide conjugates are used, the corresponding FRET oligonucleotides may contain emitter molecules which emit fluorescence that can be detected in multiple channels, e.g. they may contain emitter molecules which emit fluorescence of different wavelengths, or they may contain different emitter molecules. By way of example when groups of set of binding agent-T-oligonucleotide conjugates are used:

one or more FRET oligonucleotides may contain an emitter molecule that is Atto550, and one or more FRET oligonucleotides may contain an emitter molecule that is Atto647, or
one or more FRET oligonucleotides may contain an emitter molecule that is Atto550, and one or more FRET oligonucleotides may contain an emitter molecule that is Atto488, or
or one or more FRET oligonucleotides may contain an emitter molecule that is Atto647, and one or more FRET oligonucleotides may contain an emitter molecule that is Atto488, or
one or more FRET oligonucleotides may contain an emitter molecule that is Atto550, and one or more FRET oligonucleotides may contain an emitter molecule that is Atto488 and one or more FRET oligonucleotides may contain an emitter molecule that is Atto647.

Irrespective of the precise way in which the multiple fluorescent kinetic profiles are generated, it will be understood that by producing a group of sets of T-oligonucleotides and corresponding FRET oligonucleotides, the number of different fluorescent kinetic profiles that can be generated is increased. A greater degree of multiplexing is possible by using groups of sets of T-oligonucleotides and corresponding FRET-oligonucleotides than using a single set of T-oligonucleotides and a single corresponding FRET oligonucleotide.

Even when a group of sets of T-oligonucleotides is used, the sequence of each T-oligonucleotide that is present is unique to the binding agent to which it is conjugated. Further, the dissociation and reassociation between each different pair generates a fluorescent kinetic profile that is (i) unique within that set to that pair, and (ii) unique within the group of sets to that pair. Furthermore, in some embodiments, each T-oligonucleotide hybridises only to its corresponding FRET-oligonucleotide and does not hybridise to a FRET oligonucleotide(s) of any other set in the group of sets. Cross hybridisation should be avoided, so that the correlation between the fluorescent kinetic profile, the T-oligonucleotide and the binding agent is preserved.

Multiplexing with Multiple Groups of Sets

As discussed above, the method may comprise embodiments in which wherein step a comprises contacting the sample with multiple groups of sets of binding agent-T-oligonucleotide conjugates, each group of sets comprising a first set of binding agent-T-oligonucleotide conjugates and one or more subsequent sets of binding agent-T-oligonucleotide conjugates; and wherein the emission spectrum of the corresponding FRET oligonucleotides for each group of sets of binding agent-T-oligonucleotide can be detected in a channel that is specific to that group of sets and the step of observing the kinetic fluorescent profile of the sample comprises observing the kinetic fluorescent profile in a channel for each group of sets.

This is useful to achieve a further level of multiplexing, as compared to a single set or a group of sets. In this way the number of different channels in which the fluorescent kinetic signal is observed is increased, e.g. using FRET-oligonucleotides with fluorophores having different emission spectra, to generate fluorescent kinetic profiles that can be detected in different channels.

In such cases, each fluorescent kinetic profile that is generated can be different, however using multiple channels (i.e. emitter molecules which emit fluorescence of different wavelengths, or they may contain different emitter molecules) means that a given fluorescent kinetic profile can be duplicated between the groups of sets. In other words, two or more pairs that generate very similar or even identical fluorescent kinetic profiles can be used, if those two or more pairs that generate the similar or even identical fluorescent kinetic profiles generate fluorescent kinetic profiles as fluorescence emissions that are observed in different channels.

With this type of multiplexing, each T-oligonucleotide will still have a different sequence, that is unique to the binding agent to which it is conjugated, and each different pair in a set generates a fluorescent kinetic profile that is unique within that set to that pair. Since multiple groups of sets are being used in which the FRET-oligonucleotides from different groups of sets generate fluorescent kinetic profiles as fluorescence emissions that can be detected in different channels, this means that if a pair from one set (or one group of sets) has a similar or identical fluorescence kinetic profile to a pair from a different set (or different group of sets), both pairs can be used as long as the emitter molecules on the FRET oligonucleotides give rise to emission that can be distinguished.

Illuminating the sample at one or more wavelengths is required in order to cause excitation of each FRET-oligonucleotide's emitter molecule. Depending on the particular emitter molecule or combinations thereof that are used, it may be necessary to excite the emitter molecules using different sources of illumination e.g. different wavelengths, or a single source may be sufficient to excite all of the emitter molecules. By way of example UV illumination can be used to cause excitation of each of Atto 594, Atto 700 and Atto 465.

By way of example the FRET oligonucleotides for one group of sets of binding agent-T-oligonucleotide conjugates may contain an emitter molecule that is Atto550, and the FRET oligonucleotides for a second group of sets of binding agent-T-oligonucleotide conjugates may contain an emitter molecule that is Atto647, or

the FRET oligonucleotides for one group of sets of binding agent-T-oligonucleotide conjugates may contain an emitter molecule that is Atto550, and the FRET oligonucleotides for a second group of sets of binding agent-T-oligonucleotide conjugates may contain an emitter molecule that is Atto488, or
the FRET oligonucleotides for one group of sets of binding agent-T-oligonucleotide conjugates may contain an emitter molecule that is Atto647, and the FRET oligonucleotides for a second group of sets of binding agent-T-oligonucleotide conjugates may contain an emitter molecule that is Atto488, or
the FRET oligonucleotides for one group of sets of binding agent-T-oligonucleotide conjugates may contain an emitter molecule that is Atto550, and the FRET oligonucleotides for a second group of sets of binding agent-T-oligonucleotide conjugates may contain an emitter molecule that is Atto488 and
the FRET oligonucleotides for a third group of sets of binding agent-T-oligonucleotide conjugates may contain an emitter molecule that is Atto647.
Alternative Multiplexing with Multiple Sets

In other embodiments of the method, step a comprises contacting the sample with multiple sets of binding agent-T-oligonucleotide conjugates; and wherein the emission spectrum of the corresponding FRET oligonucleotides for each set of binding agent-T-oligonucleotide can be detected in a channel that is specific to that set. In such embodiments, more than one set of binding agent-T-oligonucleotide conjugates is used and each set of binding agent-T-oligonucleotide conjugates has a corresponding FRET oligonucleotide which gives rise to a fluorescence emission that is detectable in a single channel, e.g. each FRET oligonucleotide has an emitter molecule which emits fluorescence of a different wavelength to each other FRET oligonucleotide e.g. each FRET oligonucleotide has a different emitter molecule.

By way of example the FRET oligonucleotides for one set of binding agent-T-oligonucleotide conjugates may contain an emitter molecule that is Atto550, and the FRET oligonucleotides for a second set of binding agent-T-oligonucleotide conjugates may contain an emitter molecule that is Atto647, or

the FRET oligonucleotides for one set of binding agent-T-oligonucleotide conjugates may contain an emitter molecule that is Atto550, and the FRET oligonucleotides for a second set of binding agent-T-oligonucleotide conjugates may contain an emitter molecule that is Atto488, or
the FRET oligonucleotides for one set of binding agent-T-oligonucleotide conjugates may contain an emitter molecule that is Atto647, and the FRET oligonucleotides for a second set of binding agent-T-oligonucleotide conjugates may contain an emitter molecule that is Atto488, or
the FRET oligonucleotides for one set of binding agent-T-oligonucleotide conjugates may contain an emitter molecule that is Atto550, and the FRET oligonucleotides for a second set of binding agent-T-oligonucleotide conjugates may contain an emitter molecule that is Atto488 and the FRET oligonucleotides for a third set of binding agent-T-oligonucleotide conjugates may contain an emitter molecule that is Atto647.

### Multiplexing Using a Mixture of Sets and Groups of Sets

In other embodiments of the method a mixture of sets and groups of sets may be used. For example,

step a may comprise contacting the sample with
i. one or multiple sets of binding agent-T-oligonucleotide conjugates, wherein the emission spectrum of the emitter molecules in the corresponding FRET oligonucleotide for each set of binding agent-T-oligonucleotide can be detected in a channel that is specific to that set and
ii. one or multiple groups of sets of binding agent-T-oligonucleotide conjugates wherein the emission spectrum of the emitter molecules in the corresponding FRET oligonucleotides for each group of sets of binding agent-T-oligonucleotide can be detected in a channel that is specific to that group of sets; and
   the step of observing the kinetic fluorescent profile of the sample comprises observing the kinetic fluorescent profile of the sample in a channel for each excitation/emission spectrum.

### Kits

A kit for multiplexed fluorescence microscopy comprising: a set of binding agent-T-oligonucleotide conjugates, wherein the set comprises a plurality of binding agents having different specificities and the sequence of the T-oligonucleotide is unique to the binding agent to which it is conjugated; and a FRET-oligonucleotide, wherein the FRET-oligonucleotide can hybridise to multiple T-oligonucleotides in the set to form multiple pairs, and wherein the dissociation and reassociation between each different pair generates a fluorescent kinetic profile that is unique within that set to that pair is also provided. The components of this kit can be used to perform the method of the invention. The kit may further comprise appropriate buffers and solutions for carrying out the method steps, e.g. a fixing reagent as defined herein and/or a permeabilisation reagent as defined herein, and/or suitable solutions and liquids for washing steps.

The kit may further comprise one or more additional sets of binding agent-T-oligonucleotide conjugates, and one or more additional FRET oligonucleotide, wherein said each further FRET-oligonucleotide can hybridise to multiple T-oligonucleotides in its corresponding set, to form multiple pairs and wherein the dissociation and reassociation between each different pair generates a fluorescent kinetic profile that is unique within each set to that pair.

The kit may thus comprise one or multiple sets, or one or multiple groups of sets of binding agent-T-oligonucleotide conjugates, and a corresponding number of FRET oligonucleotides, e.g. as defined elsewhere herein in the context of the method. Each FRET-oligonucleotide can hybridise to multiple T-oligonucleotides in its corresponding set, to form multiple pairs and wherein the dissociation and reassociation between each different pair generates a fluorescent kinetic profile that is unique within each set to that pair. The emission spectrum of the FRET oligonucleotide may be specific to that set or to that group of sets and may differ between sets and groups of sets.

The invention also provides one or more sets of binding agent-T-oligonucleotide conjugates and corresponding FRET oligonucleotides for making a kit for multiplexed fluorescence microscopy comprising: one or more sets of T-oligonucleotides; and one or more FRET-oligonucleotides, wherein the sets of T-oligonucleotides; and the one or more FRET-oligonucleotides are as defined elsewhere herein.

Preferred sets of T-oligonucleotides and corresponding FRET-oligonucleotides include:
a set comprising (optionally consisting of):

one or both of the T-oligonucleotide(s) of
sequences
SEQ ID NO: 1
(5′-TTCCACATTACTTCT-3′)
and
SEQ ID NO: 2
(5′-ATCCCCATTACTTCT-3′),
and
the corresponding FRET-oligonucleotide of
sequence
SEQ ID NO: 7
(5′-AGAAGTAATGTGGAA-3′)

a set comprising (optionally consisting of):

at least one of the T-oligonucleotide(s) of
sequences
SEQ ID NO: 3
(5′-GGGTGGCGTATGATAGCTAT-3′),
SEQ ID NO: 4
(5′ -AGGTTAAGTATGATAGTTAT-3′),
SEQ ID NO: 5
(5′-GGGTTAAGCATGGTAGTTAT-3′)
and
SEQ ID NO: 6
(5′-AGGTTAAGCGTTATATTTAT-3′)
(for example at least 2, 3 or all 4 of the
recited T-oligonucleotides)
and
the corresponding FRET-oligonucleotide of sequence
SEQ ID NO: 8
(5′-ATAACTATCATACTTAACCT -3′)

The two sets above may be used together as a group of sets, e.g. where the two corresponding FRET oligonucleotides contain emitter molecules which have overlapping emission spectrums, optionally which have the same emitter molecule.

### Method of Preparing Kits

The invention also provides a method of preparing the kit referred to above, in which each set of binding agent-T-oligonucleotide conjugates and a corresponding FRET oligonucleotide is provided. The method further comprises conjugating the set of T-oligonucleotides to a set of binding agents to form a set of binding agent-T-oligonucleotide conjugates, wherein the set of binding agent-T-oligonucleotide conjugates comprises a plurality of binding agents having different specificities and the sequence of the T-oligonucleotide is unique within that set to the binding agent to which it is conjugated. The conjugation step can be performed by any suitable means known in the art, such as those described above.

### Methods of Designing a Set of T-Oligonucleotides and a FRET-Oligonucleotide for Use in the Method of the Invention

### Generation of the FRET-Oligonucleotide:

In general the FRET-oligonucleotide will be generated first, and the T oligonucleotide sequences generated subsequently. The FRET oligonucleotide will in general have a nucleotide sequence that is not complementary to any sequence that is found in the sample to which the method is to be applied. This is to avoid hybridisation of the FRET-oligonucleotide with nucleic acid material in the sample, which would give rise to fluorescence emission in the absence of binding agent-T-oligonucleotide conjugates binding to target molecules in the sample.

A suitable process for generating a FRET-oligonucleotide is set out below. An initial sequence is selected from randomly generated sequences with simple to more complex combinations of nucleotides and that are pre-filtered to not match any known genomic sequence. A suitable sequence will provide enough flexibility in the FRET-oligonucleotide's unbound state for the terminals to be in close proximity, for example in order to participate in electron transfer or electron exchange.

Because there is a need to have variety in the fluorescent kinetic profiles of pairs used in the method, the length of the FRET oligonucleotide can be selected on the basis of the desired length of time that it will stay in a bound state (i.e. associated with a T-oligonucleotide); extending the FRET-oligonucleotide's length will strengthen base-pairing and dissociation will therefore take longer or require more energy. Increasing its GC content will strengthen base-pairing and dissociation will therefore take longer or require more energy.

Once a suitable sequence has been generated for the FRET-oligonucleotide in silico, the sequence is tested to confirm the absence of secondary structure, and the lack of possible self-ligation, and/or cross-dimerisation with other FRET-oligos that are to be used in the method. This can be carried out by methods known in the art, such as the standard software tools provided by most oligonucleotide vendors. Examples of commercially available tools include e.g. Sigma-Aldrich ‘Oligo Analyzer Tool’, Thermofisher ‘Multiple Primer Analyzer’, IDT ‘OligoAnalyzer™ Tool’.

### Generation of the T-Oligonucleotides:

Once a FRET-oligonucleotide sequence has been generated, suitable T-oligonucleotides can then be generated. In general this is carried out by starting with a sequence that is fully complementary to all or a portion of the FRET-oligonucleotide.

Possible T-oligonucleotides are generated in silico by introducing mismatches into the fully complementary sequence. For example the mismatches may be introduced at 20 to 40% of the possible base pairs. Mismatches may be introduced in different patterns as discussed above.

Possible fluorescent profile kinetics for a given pair can be inferred from the number and location of mismatches (e.g. a pair in which there is a run of mismatches will have a different profile to a pair having the same number of mismatches but equally distributed throughout the sequences). Sequences that are likely to give rise to a required fluorescence kinetic profile are synthesised and tested for their blinking profiles.

This is shown, for example in FIG. 2A and FIG. 2B. Fluorogenic profiles of FRET/T-oligo pairs can be assessed in solution. A FRET-oligonucleotide is contacted with the different T-oligonucleotides in solution and the fluorescence emission is measured at different concentrations of the T-oligonucleotides, and optionally compared to the fully matched sequence. As can be seen in FIG. 2A a fully matched oligonucleotide generates a different profile to the mismatched T-oligonucleotides, and the different mismatched pairs have different fluorogenic profiles in solution. These distinct kinetics profiles are generated when the FRET-oligo is in the presence of different T-oligonucleotides that contain mismatches to interfere with the proper base pairing between the FRET-oligo and the T-oligonucleotide. The hybridisation of the FRET-oligonucleotide to each T-oligonucleotide generates a distinct fluorogenic profile with the more disruptive mismatches leading to a seemingly reduced fluorogenicity.

By testing in solution, only the overall amount of fluorescence can be measured. Further information on the profile can be generated by adding the FRET-oligonucleotide to the same T-oligonucleotides—this time immobilised on a surface (mimicking what will occur in the method of the invention when the T oligonucleotide is conjugated to a binding agent which in turn will bind to the sample so that the T oligonucleotide is effectively immobilised on the sample). The FRET-oligo in this configuration will emit intermittent fluorescence. FIG. 2B shows representative outcomes for tested oligonucleotide pairs, with the number of blinking events per second being shown per pair, (e.g. the number of occurrences of fluorescence emission within a designated time frame of second). As discussed above, the number of occurrences of fluorescence emission within a designated time frame is one component of the fluorescent kinetic profile. The blinking kinetics are influenced by factors including the nature of the mismatches introduced in the T-oligonucleotides such as disruptive mismatches that reduce the fluorogenic profile of the FRET-oligonucleotide. As shown in FIG. 2B the different pairs have different numbers of occurrences of fluorescence emission within a designated time frame.

The ability to observe the fluorescent kinetic profile of any given pair in this test format means that when designing the pairs, appropriate sequences can be chosen to provide the range of profiles that is required for a given application.

The invention therefore provides a method of designing a set of T-oligonucleotides and a FRET-oligonucleotide for use in the method of the invention, said method comprising:
a. selecting a FRET-oligonucleotide sequence of at least 12 nucleotides in length;
b. Obtaining one or more sequences of at least 8 nucleotides in length that are complementary to the FRET-oligonucleotide sequence;
c. Generating a plurality of potential T-oligonucleotide sequences each of which differs from the sequence that is complementary to the FRET-oligonucleotide by at least one nucleotide;
d. Selecting at least two T-oligonucleotide sequences on the basis of the ability of the at least two T-oligonucleotide sequences to generate fluorescent kinetic profiles that are different to each other.

Step a, selecting a FRET-oligonucleotide sequence of at least 12 nucleotides in length, may be carried out as described above, and may include one or more of (i) determining whether the potential FRET oligonucleotide has a nucleotide sequence that is not complementary to any sequence that is found in the sample to which the method is to be applied, (ii) determining whether said sequence has enough flexibility in the FRET-oligonucleotide's unbound state for the terminals to be in close proximity, for example in order to participate in electron transfer or electron exchange, (ii) determining whether the potential FRET oligonucleotide has a nucleotide sequence that is not complementary to any sequence of another FRET oligonucleotide that is to be used in the method of the invention.

Step b, obtaining one or more sequences of at least 8 nucleotides in length that are complementary to the FRET-oligonucleotide sequence is simply carried out by producing the complementary pair. Since a FRET oligonucleotide is in general at least 12 nucleotide units in length and the T oligonucleotide may be shorter, this step may entail making multiple possible complementary sequences of different lengths.

Step c, generating a plurality of potential T-oligonucleotide sequences each of which differs from the sequence that is complementary to the FRET-oligonucleotide by at least one nucleotide, like step b, can be carried out in silico or manually.

Step d, selecting at least two T-oligonucleotide sequences on the basis of the ability of the at least two T-oligonucleotide sequences to generate fluorescent kinetic profiles that are different to each other, may be carried out by inferring these properties from the sequences, e.g. the nature, number and location of any mismatches, or by synthesising the molecules and testing them. The step of testing them may involve contacting the members of each pair in solution, and observing the fluorescence emission, e.g. determining the amount of emission. This may be carried out at more than one concentration of potential T-oligonucleotide, e.g. to generate a fluorogenic profile as shown in FIG. 2A. In addition, the potential T oligonucleotides may be immobilised on a solid surface and contacted with the FRET oligonucleotide, and the fluorescent kinetic profile of each pair observed.

At least two T oligonucleotides are then selected to form a set. These T oligonucleotides give rise to a fluorescent kinetic profile that is unique within the set.

The same principles can be applied with designing FRET-oligonucleotides to be included in a second or subsequent set or for use in a group of sets. Additional considerations for a second or subsequent set include (i) the need for the FRET-oligonucleotide(s) to not hybridise to any T-oligonucleotides in other sets and (ii) to not hybridise to other FRET-oligonucleotides.

Preferred sets of T-oligonucleotides and corresponding FRET-oligonucleotides include:
a set comprising (optionally consisting of):

one or both of the T-oligonucleotide(s) of
sequences
SEQ ID NO: 1
(5′-TTCCACATTACTTCT-3′)
and
SEQ ID NO: 2
(5′-ATCCCCATTACTTCT-3′),
and
the corresponding FRET-oligonucleotide of
sequence
SEQ ID NO: 7
(5′-AGAAGTAATGTGGAA-3′)

a set comprising (optionally consisting of):

one or more T-oligonucleotide(s) of sequences
SEQ ID NO: 3
(5′-GGGTGGCGTATGATAGCTAT-3′),
SEQ ID NO: 4
(5′-AGGTTAAGTATGATAGTTAT-3′),
SEQ ID NO: 5
(5′-GGGTTAAGCATGGTAGTTAT-3′)
and
SEQ ID NO: 6
(5′-AGGTTAAGCGTTATATTTAT-3′)
and
the corresponding FRET-oligonucleotide of
sequence
SEQ ID NO: 8
(5′-ATAACTATCATACTTAACCT-3′)

### General

The term “comprising” encompasses “including” as well as “consisting” e.g. a composition “comprising” X may consist exclusively of X or may include something additional e.g. X+Y.

The term “about” in relation to a numerical value x means, for example, x±10%.

The word “substantially” does not exclude “completely” e.g. a composition which is “substantially free” from Y may be completely free from Y. Where necessary, the word “substantially” may be omitted from the definition of the invention.

Where the invention provides a process involving multiple sequential steps, the steps are carried out in the indicated order, i.e. in numerical or alphabetical order. However, the skilled person will understand that the order of steps may be altered while still achieving useful results. The invention can also provide a process involving less than the total number of steps.

### BRIEF DESCRIPTION OF DRAWINGS

FIG. 1A and FIG. 1B show—DNA-based stochastic fluorescence using Forster Resonance Energy Transfer oligonucleotide (FRET oligonucleotide)-T-oligonucleotide pairs. (FIG. 1A) When the FRET-oligonucleotide is in solution, upon illumination with the emitter's excitation wavelength, no fluorescence is observed due to efficient energy transfer to the quenching entity. (FIG. 1B) However, upon FRET-oligonucleotide hybridisation with an immobilised T oligonucleotide, the emitter and quenching entity are separated physically and the rate of FRET is reduced, allowing the emission from the emitter fluorophore to be detected at the site of hybridisation.

FIG. 2A and FIG. 2B show Fluorogenic and blinking profiles of FRET/T-oligo pairs. (FIG. 2A) Fluorogenic profiles of FRET/T-oligo pairs are assessed in solution—Distinct fluorogenic profiles are generated when the FRET-oligo is in the presence of different T-oligonucleotides that contain mismatches to interfere with the proper base pairing between the FRET-oligo and the T-oligonucleotide. The hybridisation of the FRET-oligonucleotide to each T-oligonucleotide generates a distinct fluorogenic profile with the more disruptive mismatches leading to a seemingly reduced fluorogenicity. (FIG. 2B) When the FRET-oligonucleotide is added to the same T-oligonucleotides—this time immobilised on a surface or in a cell via antibody-conjugation-intermittent fluorescence of the FRET-oligonucleotide can be observed directly. The blinking kinetics are influenced by factors including the nature of the mismatches introduced in the T-oligonucleotides such that disruptive mismatches that reduce the fluorogenic profile of the FRET-oligonucleotide (FIG. 2A) increase its blinking rate (FIG. 2B).

FIG. 3A-FIG. 3D show examples of different fluorescent kinetic profiles.

FIG. 4A and FIG. 4B show a representation of emission signals from immobilised T-oligonucleotide+FRET-oligonucleotide binding in a cellular sample. (FIG. 4A) Primary antibodies that recognize specific cellular components structures are covalently attached to distinct T-oligonucleotides and used to immunolabel a fixed, permeabilized cell. The transient hybridization of a FRET-oligonucleotide results in intermittent fluorescence at a given foci. (FIG. 4B) By distinguishing the kinetics of binding hybridisation (and thus fluorescence), the identity of each foci can be assigned to one of dozens of FRET-oligo/T-oligonucleotide pairs, or to background.

FIG. 5A-FIG. 5D show Channel image construction given T-oligonucleotide deconvolution. (FIG. 5A) A cell sample is labeled with antibodies recognising distinct molecular epitopes. Each antibody species is conjugated to a distinct T-oligonucleotide. Next, the FRET-oligonucleotide corresponding to the conjugated T-oligonucleotides are added to the sample. Each FRET-oligonucleotide pair employs the same emitter fluorophore and fluorescence quencher. When unbound from the T-oligonucleotides, the FRET-oligonucleotide fails to fluoresce due to FRET-based quenching. When hybridised with its corresponding T-oligonucleotides, fluorescence occurs at the site of hybridisation. Longitudinal image acquisition allows each pixel to be measured for fluorescence, and a profile to be created. (FIG. 5B) As each FRET-/T-oligonucleotide pair has a unique kinetic binding profile, this can be used to link a pixel's fluorescence profile to its T-oligonucleotide identity, therefore cellular target. (FIG. 5C) Each pixel is assigned to a particular T-oligonucleotide identity, or to background. (FIG. 5D) From this assignment, separate channel images for each imaging modality can be generated, despite the experiment using a single laser excitation wavelength.

FIG. 6 shows dose-response of a Cy5-BHQ-3 FRET-oligonucleotides in the presence of different T-oligonucleotides. Fluorescence intensity was averaged across 3 replicate using either the total or average number of raw pixels computed across a stack of 50×10 ms images captured ˜10 μm above the bottom of the well. Values were normalised to the average fluorescence values obtained for the Cy5-labeled control oligo (i.e. unquenched emitter). For the FRET-oligo/fully matched T-oligo pair (0 mismatch), mean values were computed across all samples plates. Shown here are the plots aggregated by number of mismatches with each shaded area encompassing the 95% CI for the different profiles obtained within each group.

FIG. 7A and FIG. 7B show a broad range of fluorogenic profiles is obtained for the FRET-oligo in presence of T-oligos with 3 mismatches. This suggests that mismatches are not equal with some being more costly than others. For each hybridisation, position and base substitution are indicated.

FIG. 8A and FIG. 8B show introduction of 4 mismatches in the T-oligo has generally a higher cost and dramatically impact the FRET-oligo's fluorogenic profile (dose response curves which remain below 0.1 normalised fluorescence). However, the FRET-oligo seems to hybridise better to a few selected T-oligos (curves reaching a normalised fluorescence of 0.1 to 0.2). Comparatively, a fast-blinking pair with a T-oligo containing 4-mismatches (dotted line) lies among the poorly fluorogenic ones.

FIG. 9A and FIG. 9B show introduction of a single mismatch in the T-oligo affects the fluorescence profile in solution of the FRET-oligo differently depending on the position of the mismatch and its identity. For each hybridisation, position and base substitution are indicated. The fluorescence profile obtained when the FRET-oligo is incubated with increasing concentrations of a fully complementary T-oligo is included. Hybridisation partners are shown with distinct mismatches albeit at the same position. Comparison of substitutions at position 14 for instance shows that substituting T for A has virtually no impact on the FRET-oligo's fluorogenicity while a T to C substitution leads to a drop in fluorogenicity.

FIG. 10 shows Maximum intensity Z projections for complementary ‘matched’ and mismatched T-oligos showing the cumulative number and distribution of blinking events over the total acquisition period of 40 s. Increased number of spots indicates increased number of blinking events. The mismatched T-oligonucleotide (right) produces more blinks over the course of exposure than the matched T-oligonucleotide (left).

FIG. 11 shows workflow describing the fluorescence kinetic profiling of the FRET-oligo paired with distinct T-oligos. For each experimental group, 2000 images were acquired at a frame rate of 20 ms, giving a total acquisition time of 40 seconds. Time traces of fluorescence intensity (boxed traces) show the kinetic profile of specific regions of interest in the image stack (to which they are linked). The histogram plot on the right shows the cumulative frequency of blinking events/frame over the total acquisition time for two distinct T-oligo designs.

FIG. 12A and FIG. 12B show focal kinetic profiles obtained upon addition of the FRET-oligo to immobilised T-oligos on biotin/streptavidin-coated glass coverslips. For each measurement, individual foci were recorded across 1000×20 ms exposures. Sample fluorescent kinetic profiles are shown for the fully matched T-oligonucleotide (FIG. 12A) and the mismatched T-oligonucleotide (FIG. 12B). The average number of blinking events during the total duration of exposure is lower for the matched T-oligonucleotide (1.8) compared to the mismatched T-oligonucleotide (4.6). Moreover the average on-time for a given blinking event is greater (95 ms) for the matched T-oligonucleotide versus the mismatched (30 ms). These are two exemplary metrics that could be used to deconvolute the T-oligonucleotide identity.

FIG. 13 shows Spectral Independent Multiplexed Imaging SIMI of mismatched and complementary ‘matched’ T-oligos upon addition of FRET-oligo. T-oligos-coated beads in the presence of 100 nM of FRET-oligo. Image represents a maximum intensity Z projection of 1000 10 ms exposures. Circles indicate beads with complementary (A) and mismatched (B) T-oligos and their corresponding kinetic profile (right panel+). Circle C indicates an equal area region used to measure background. The average signal measured within the circled areas are plotted with respect to time in the exposure. The beads coated with the mismatched T-oligonucleotide exhibit less overall fluorescence than the beads coated with the matched T-oligonucleotide. As this is the same sample, using a single FRET-oligonucleotide, this difference is attributable to the shorter on-time of blinking in the mismatched T-oligonucleotide.

FIG. 14A-FIG. 14D show Spectral Independent Multiplexed Imaging (SIMI) of 6 cellular targets on fixed cells. Targets were imaged by immobilising 6 distinct antibodies on fixed cells, each conjugated to a different T-oligo derived from two different sets of T-oligos. Two FRET-oligos, each associated to one of the set of T-oligos were added simultaneously and diluted into imaging buffer at 5 nM. 10 000 frames of 10 ms exposure each were acquired on a Nikon 60×1.49NA TIRF objective, filtered through a Cy5 filter set and imaged using a prime bsi express CMOS camera.

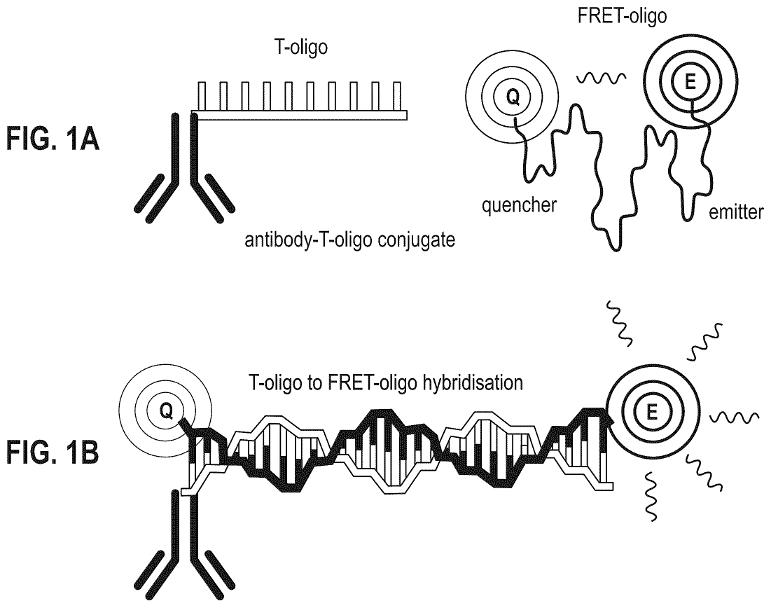

Super-resolution images were reconstructed from the cumulated frames using Picasso and QC-STORM softwares. Demultiplexing of the super-resolved images was performed using metrics derived from each FRET-oligo/T-oligo associated fluorescent kinetic profile.

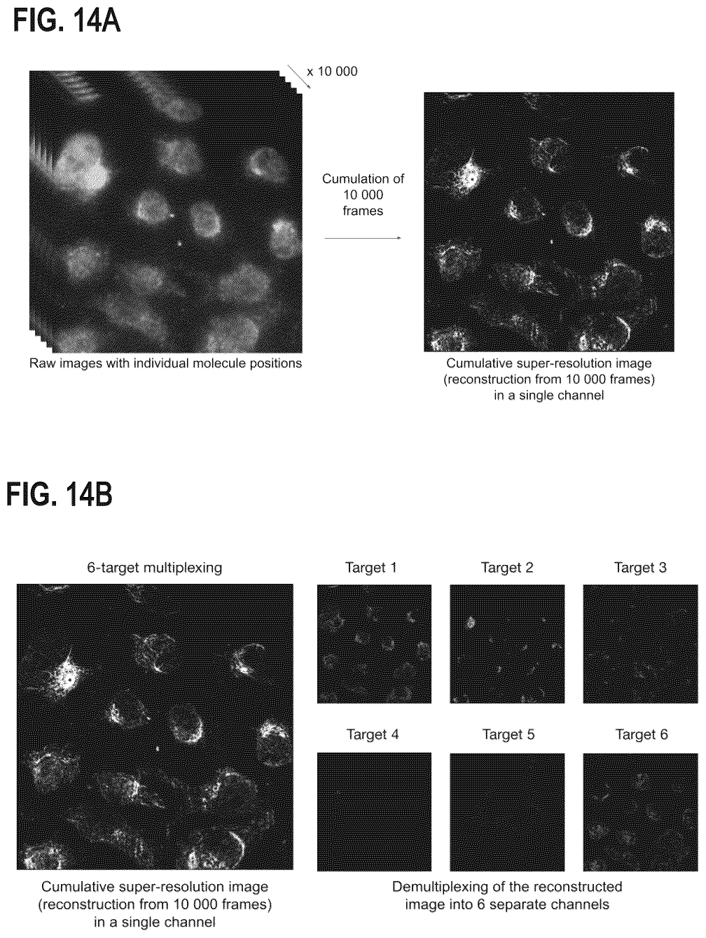

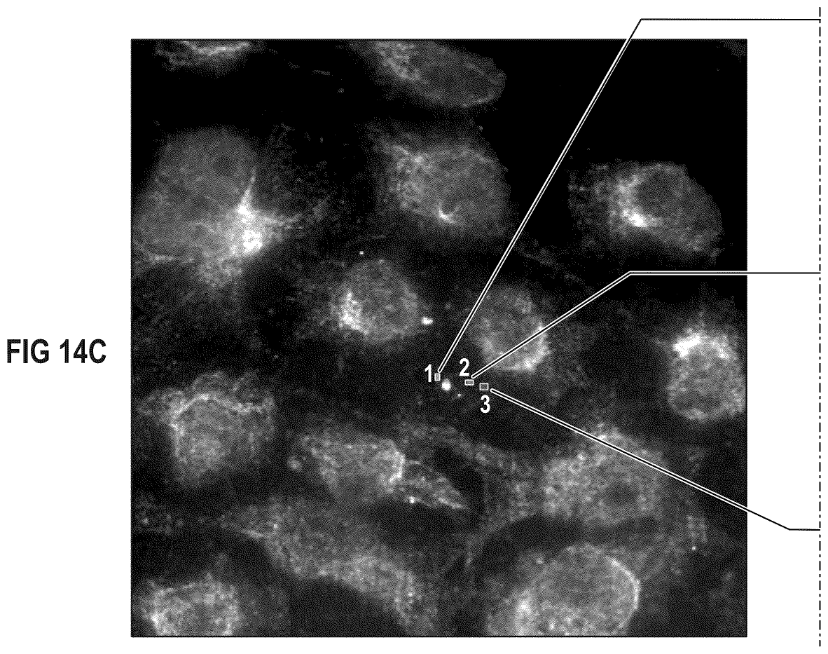

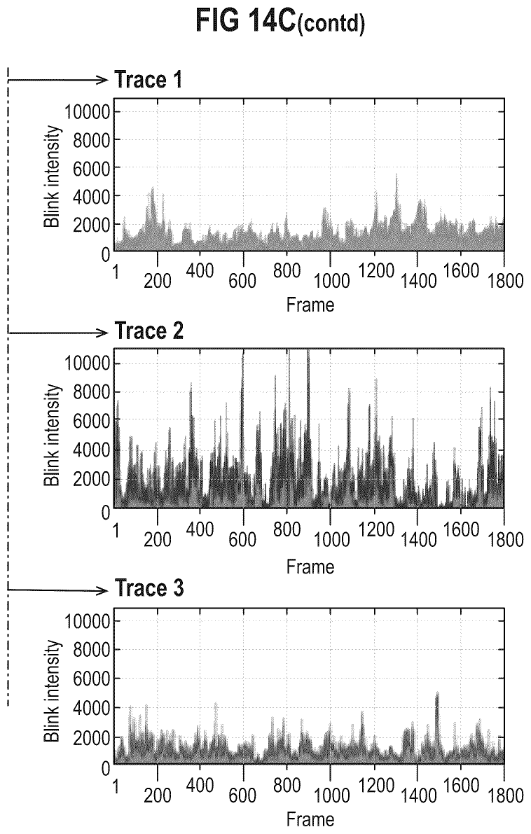

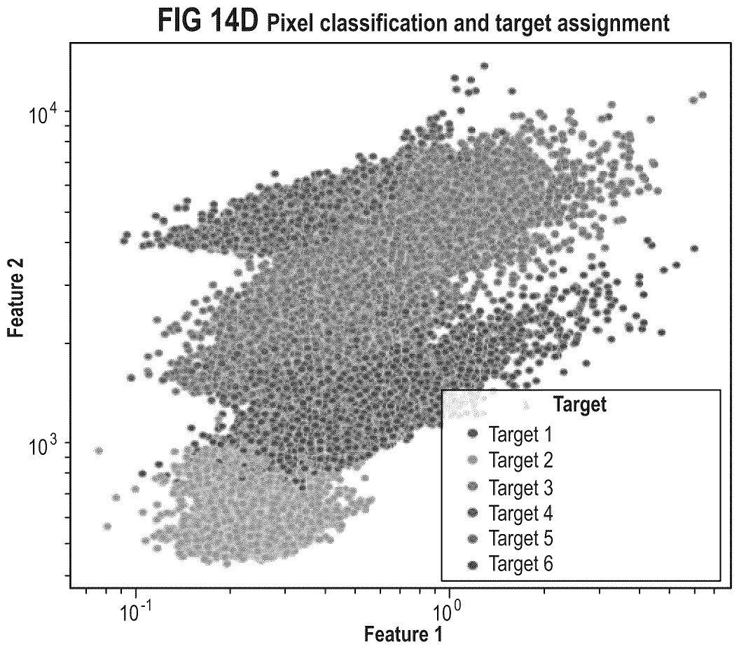

(FIG. 14A) A super-resolution image with the spatial positions of individual molecules is reconstructed from thousands of raw images (forming a longitudinal series of images). (FIG. 14B) Demultiplexing of the reconstructed image enables individual cellular targets to be assigned into separate channels. The 6 individual gray scale channel images are shown and localise the 6 different cellular targets on the sample. (FIG. 14C) Pixels are assigned to a FRET-oligo/T-oligo pair (and hence to a cellular target) using pixel-wise relevant metrics that are computed across a defined number of frames. Some of these metrics may be computed from temporal traces (fluorescent kinetic profiles) as shown here for 3 exemplary pixels. (FIG. 14D) Features including spatial-temporal information that are extracted from each pixel can be plotted to classify pixels and perform target assignment (demultiplexing).

### MODES FOR CARRYING OUT THE INVENTION

### Example 1

T-oligos are covalently attached to various antibodies that recognize different epitopes within the cell. Cells are fixed and permeabilised with standard cell biology protocols. Primary antibody-T-oligo conjugates are contacted with the fixed, permeabilized sample bind to respective epitope sites. Cells are washed of unbound antibody-T-oligo conjugates. A FRET-oligo is then incubated with the sample, under illumination at the emitter fluorophore's excitation wavelength. When the FRET-oligo is not hybridised with its respective T-oligos, the quenching entity suppresses the fluorescence emission from the emitter fluorophore. Upon hybridisation, this quenching is released, allowing detection of fluorescence at the site of hybridisation.

A high-speed CMOS sensor acquires images on the sample over a matter of seconds to minutes. Each foci is monitored for fluorescence over time, and a kinetic fluorescence profile is generated. The fluorescence profiles (or metrics thereof) at a specific foci are used to determine that foci's fluorescent kinetic profile.

Each pixel's identity is assigned to one of the FRET-/T-oligo pairs, or to background.

Pixels for each FRET-/T-oligo pair are separated into individual channels, and represent dozens of different imaging modalities captured under the same illumination wavelength of light.

### Example 2

### Example 2—Generation of FRET-Oligonucleotide to Transactivating-Oligonucleotide Pairs with Distinct Focal Kinetic Profiles for Use in Spectral Independent Multiplexed Imaging (SIMI)

### Introduction

The aim is to develop a platform for multiplex imaging by super-resolution microscopy using probes termed FRET-oligonucleotides or FRET-oligos. These FRET-oligos are designed to enable transient binding to designated DNA targets and consequential switching between visible and invisible states (i.e. blinking). The design leverages FRET (Förster resonance energy transfer)-mediated quenching, with the ends of the FRET-oligo conjugated with a quencher such as a non-fluorescent quencher acceptor and an emitter (e.g. fluorophore donor). The nucleotide sequence of the probe provides high structural flexibility allowing the emitter and quencher to be in close proximity leading to strong quenching and suppression of background fluorescence. Binding of the FRET-oligo to a Transactivating oligonucleotide or T-oligo causes a change in conformation that prevents energy transfer from the donor to the acceptor and results in the detection of donor fluorescence.

Spectrally independent multiplexed imaging (SIMI) can, in principle, image an unlimited number of sub cellular targets at nanometer length scales. SIMI using FRET-oligo/T-oligo pairs aims to kinetic profile the blinking rates associated with different oligonucleotide sequence designs. This could enable multiple target markers to be identified within a single super-resolution image acquisition, leading to a massive upscale in the phenomic profiling capacity of FRET-oligo/T-oligo-based SIMI. To realise this goal, the kinetic profiles of the different FRET-oligo/T-oligo pairs need to be sufficiently distinct from one another to be accurately identified in the same field of view.

### Material and Methods

### Oligos

The exemplary FRET-oligo is a 15-nucleotide long sequence (5′ AGAAGTAATGTGGAA (SEQ ID NO:7)) that we derived from Chung et al. BioRxiv 2020 (Chung, K. KH, Zhang, Z., Kidd, P., Zhang, Y., Williams, ND., Rollins, B., Yang, Y., Lin, C., Baddeley, D. and Bewersdorf, B. (2020). Fluorogenic probe for fast 3D whole-cell DNA-PAINT. BioRxiv), that is conjugated at the 5′ and 3′ end with a Cy5 fluorophore and a BHQ-3 dark quencher, respectively. We generated the T-oligos by introducing mismatches at random positions using a Python script. All oligos (i.e. FRET-oligo and T-oligos) were purchased from IDT and resuspended in 100 μM TE buffer (10 mM Tris-HCl and 1 mM EDTA, pH 8.0). Aliquots were prepared to limit the number of freeze-thaw cycles and stored at −20° C.

### Fluorogenic Profiling

T-oligos at 100 μM, 10 μM or 1 μM were prepared in TE buffer and diluted to 5, 50, 500 or 5000 nM in imaging buffer (1×PBS with 500 mM NaCl). The FRET-oligo was used at a final concentration of 50 nM in a 50 μL total volume.

Buffer alone, Cy5- or BHQ-3 labeled oligonucleotide alone or FRET-oligo alone were used as controls. Furthermore, samples with the FRET-oligo incubated with increasing concentrations as defined above of its fully complementary T-oligo were used as additional controls and to define the FRET-oligo's intrinsic fluorogenic profile.

Fluorescence emission from the probe was measured using an Olympus BX50 inverted microscope. Samples were illuminated using a mercury arc lamp filtered through a 530-550 nm bandpass filter. The fluorescence emission from the sample was passed through a 590 nm longpass filter and imaged onto a sCMOS camera chip. 50 images of 10 ms exposures were acquired (500 ms second integration time) using a 40×0.6NA objective for each well, and pixel intensities were averaged or summed across the stacked images using image J. Each sample was loaded in triplicate in 96-well plates.

Background fluorescence from imaging wells with buffer or BHQ-3-labeled oligo only (the values were equivalent) was subtracted from the fluorescence intensity values obtained for each sample. Reported values were computed as an average across the 3 replicates and normalised to the fluorescence intensity values obtained with the Cy5-labeled oligo (i.e. unquenched emitter).

### SIMI

### T-Oligo Blinking Profiling

For each T-oligo, 1 μM of biotinylated T-oligo was added to BSA-biotin-streptavidin coated glass coverslips mounted on 18 well Ibidi sticky slides and incubated either for 1 hr at 37° C. or 4° C. overnight. Washed coverslips were then immersed in 50 μL of imaging buffer (TE buffer with 500 mM NaCl) and placed under the microscope. 10 nM of the Cy5/BHQ-3 FRET-oligo Fret probe was then added to the sample. Following excitation with a 639 nm laser running a full power (150 mW), fluorescence was collected using a Nikon 60×1.49NA TIRF objective, filtered through a Cy5 filter set and imaged using a prime bsi express CMOS camera. Image analysis and post-processing of the acquired images was performed in ImageJ and single particle detection using the open source plugin trackmate.

### T-Oligo Focal Kinetic Profiling

5 μL of streptavidin-coated polystyrene beads (0=3 μm) were resuspended in 50 μL of binding/wash Buffer (20 mM Tris, pH 7.5; 1 M NaCl, 1 mM EDTA). Washed beads were then mixed with 1 μM of biotinylated T-oligos, and incubated overnight at 4° C.

1 μL of a mixture of mismatched and fully-complementary T-oligo-conjugated beads was immobilised on BSA/biotin-coated glass coverslips in a 18-well Ibidi sticky slide and imaged with the same microscope setup as above in presence of 100 nM of FRET-oligo.

Stacks of 1000 10 ms exposures were used to generate blinking kinetic profiles for fast and matched T-oligos.

### Results

We designed our FRET-oligo using the oligonucleotide sequence published by Chung et al. BiorXiv 2019 and by labeling the 5′ and 3′ ends with Cy5 and BHQ-3, respectively, which is a dye/quencher pair commonly used in FRET experiments.

We measured the fluorescence in solution of the Cy5/BHQ-3 FRET-oligo in presence of increasing concentration of devolved T-oligos. To generate these, we designed a Python script that modified 1-6 nucleotides at random positions in the fully complementary T-oligo. 15 T-oligos for each category of 1-6 mismatches were tested for emission of fluorescence upon binding to the FRET-oligo.

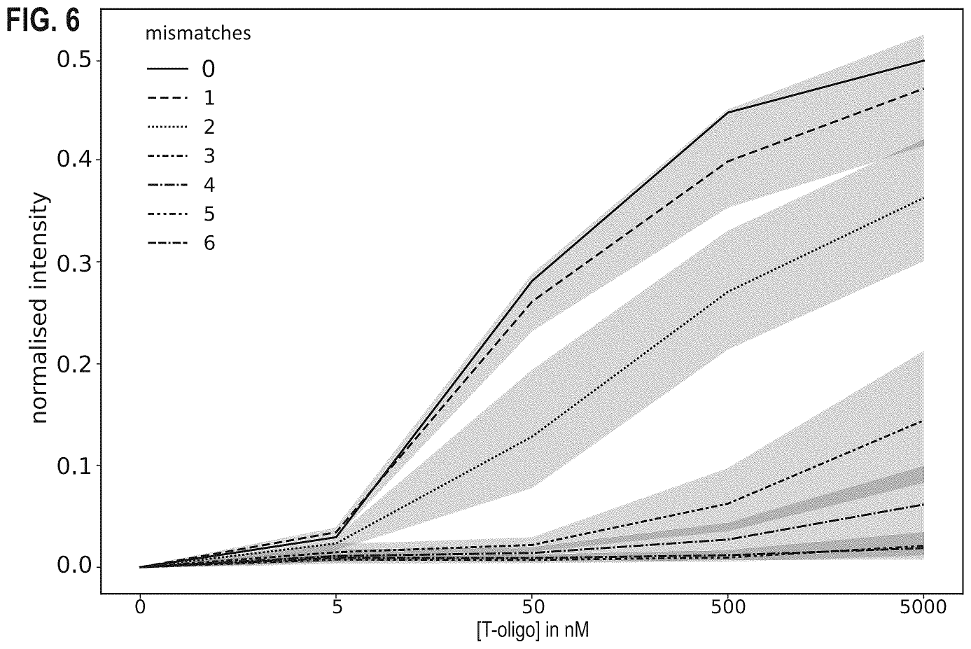

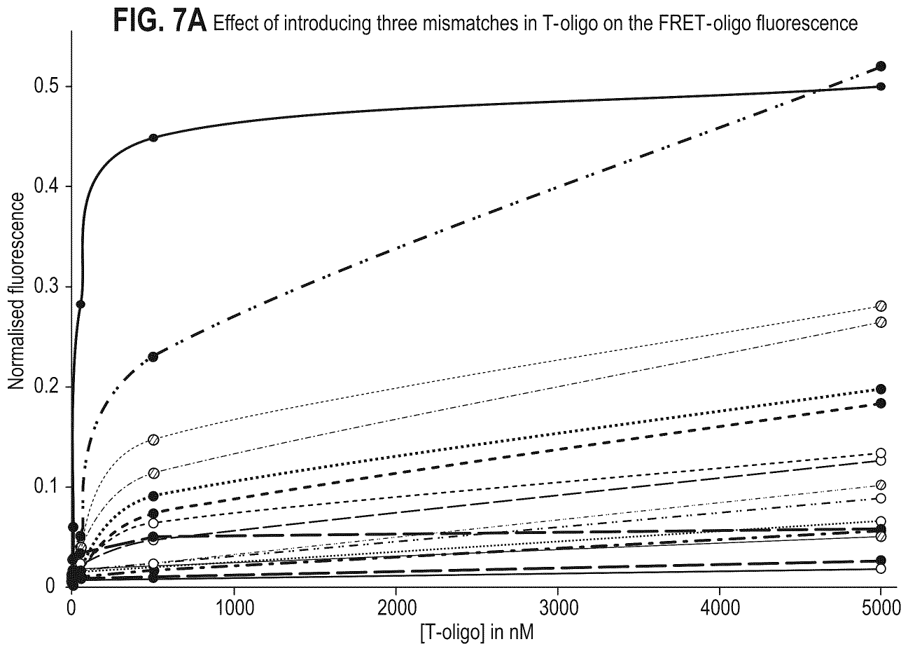

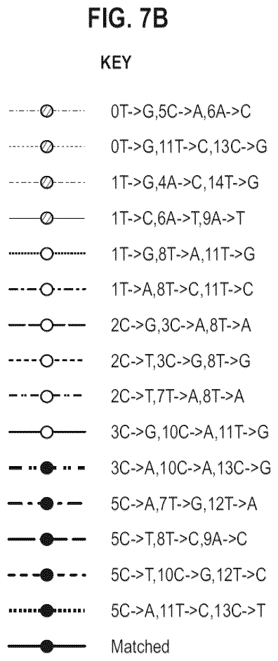

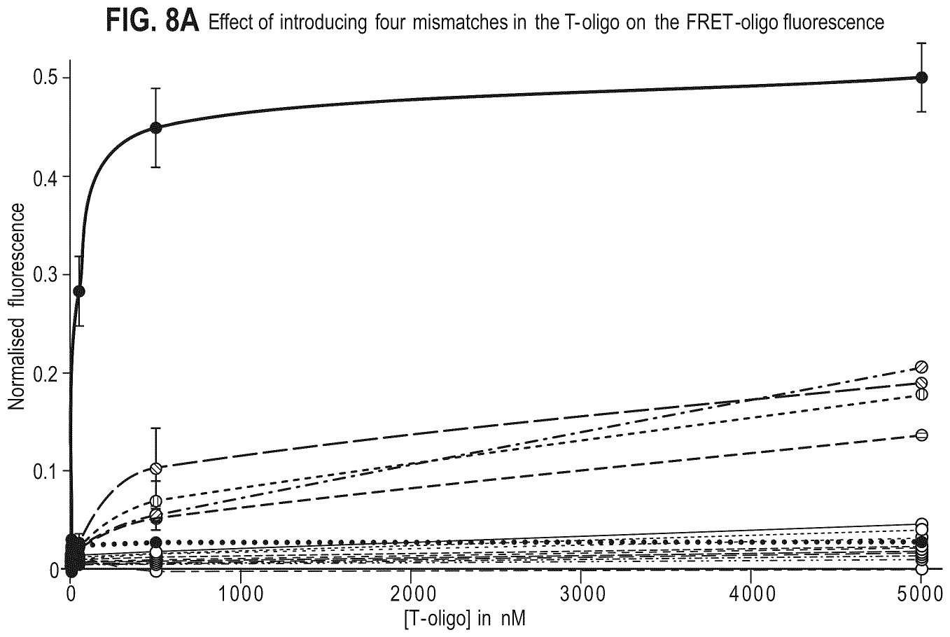

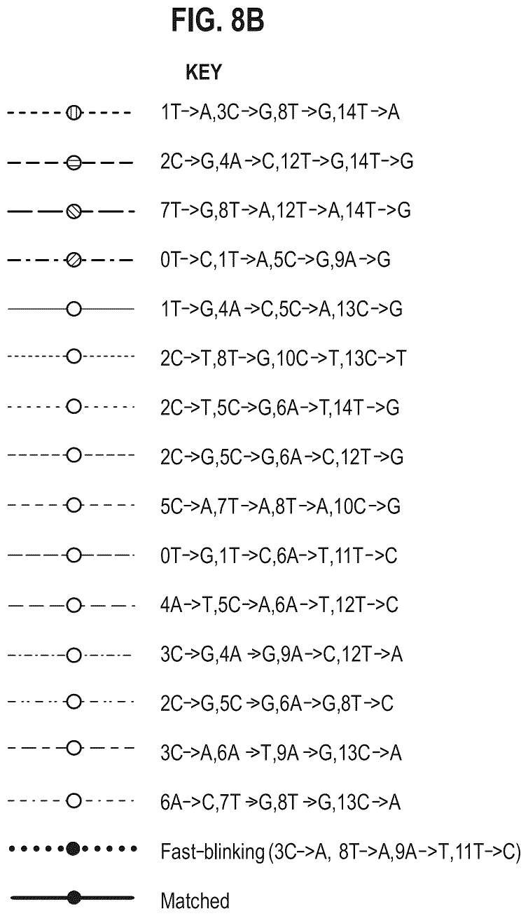

We observed that there was a general correlation between the number of mismatches introduced in the hybridisation partner and the dose-response curve obtained for each probe/partner pair (FIG. 6), such that the more mismatches introduced the lower the probe fluorogenicity and Saturated Fluorescence Level (SFL) value. Furthermore, with a few notable exceptions, T-oligos with 2 or more mismatches displayed an activation concentration 50 (AC50) 3 to 40-fold higher than that of the fully complementary T-oligo, suggesting that higher concentrations of partially complementary hybridisation partners were required to activate the FRET-oligo. In general, introduction of 5 or more mismatches drastically decreased the FRET-oligo probe's fluorogenicity, possibly abrogating it entirely in some cases (FIG. 6). The broadest range of fluorogenic profiles was obtained with the introduction of 3 mismatches (FIG. 7A and FIG. 7B), while a few T-oligos with 4 mismatches seemed to perform better than the majority (FIG. 8A and FIG. 8B).

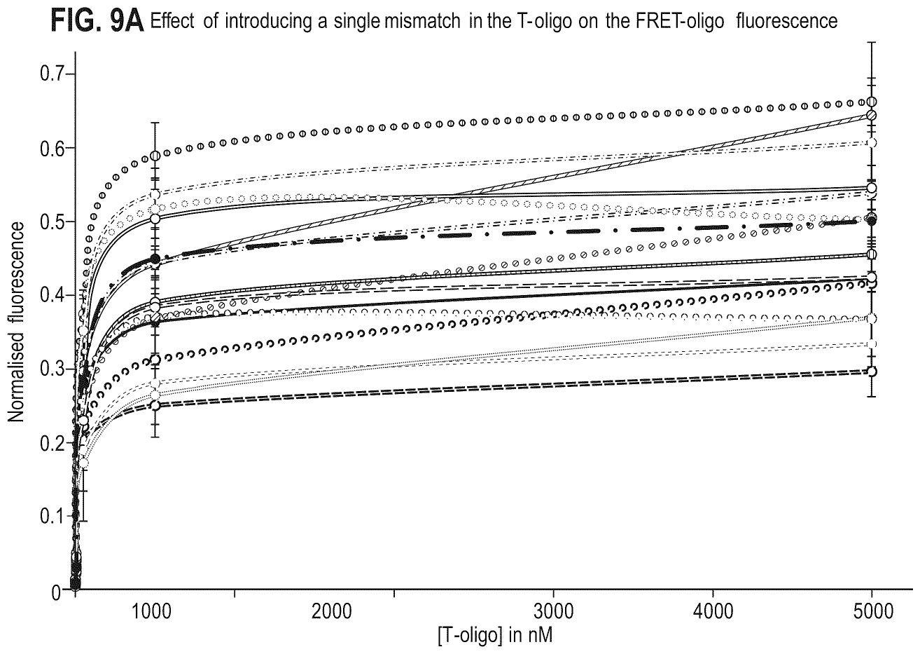

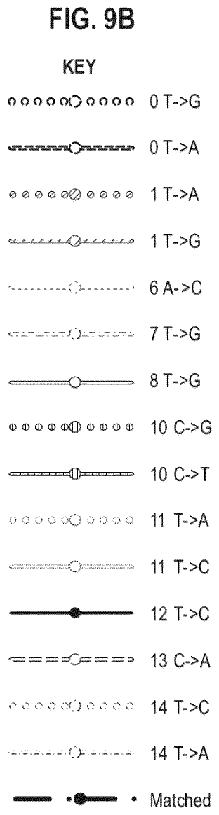

To further investigate whether specific mismatches could explain the observed differences in fluorogenic profiles, especially for T-oligos with ≤4 mismatches, we analysed the position and identity of the substitutions. Analysis of the T-oligos with a single mismatch with the FRET-oligo probe showed that certain positions affected the dose-response and SFL more than others (FIG. 9A and FIG. 9B). In general, the first residue and residues centrally located in the sequence seemed to be important for binding of the FRET-oligo to the T-oligo, while downstream positions to a lesser extent. Comparison of mismatches at similar position but with distinct substitutions suggested that some residues might be more detrimental to the FRET-oligo/T-oligo hybridisation and emission of fluorescence than others.

In the next step, we selected a set of T-oligos with distinct fluorogenic profiles in order to validate the hypothesis that a correlation exists between the fluorogenicity of a FRET-oligo/T-oligo pair and its blinking characteristics.

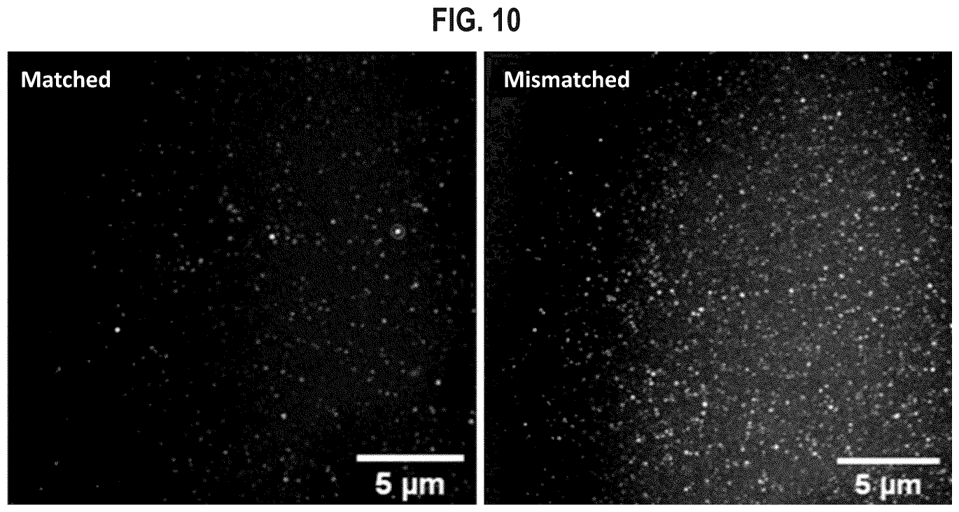

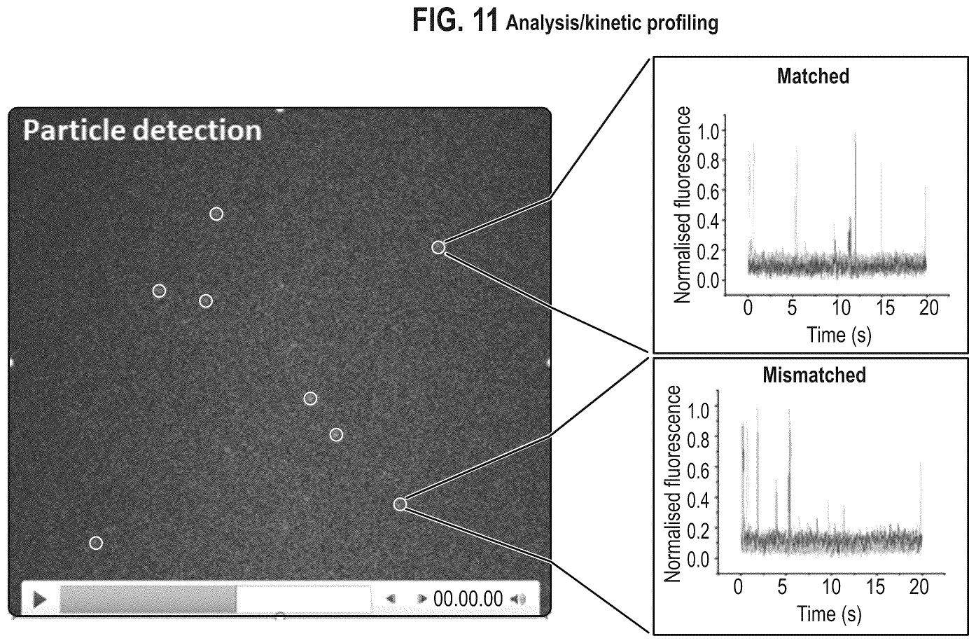

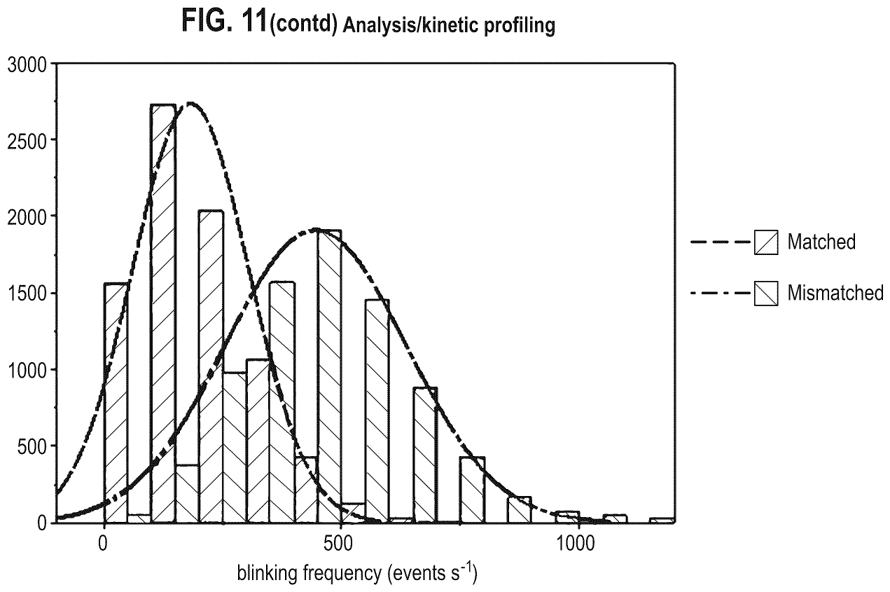

To establish a robust measurement protocol for recording blinking events, we initially studied two T-oligo designs: a fully complementary T-oligo denoted ‘matched’ and a T-oligo with 4 nucleotide mismatches denoted ‘mismatched’. Image stacks consisting of 2000×20 ms exposures were acquired in three different regions of each well containing surface-immobilised T-oligos. Analysis of the maximum intensity z-projection of 2000×20 ms camera exposures for the two T-oligos revealed a qualitative change in density of events with a significant increase in the number of events in the 4 mismatched T-oligo when compared with the fully complementary T-oligo (FIG. 10). By selecting a region of interest and measuring the fluorescence intensity across the image stacks (FIG. 11) we visualised individual time traces for regions of interest where blinking occurs (green and purple line plots). Analysis of the cumulative distribution of the total number of events detected per frame demonstrated that as the frequency of blinks/frame increases the distribution shifts to the right, allowing distinct kinetic profiles to be identified for the two T-oligos.

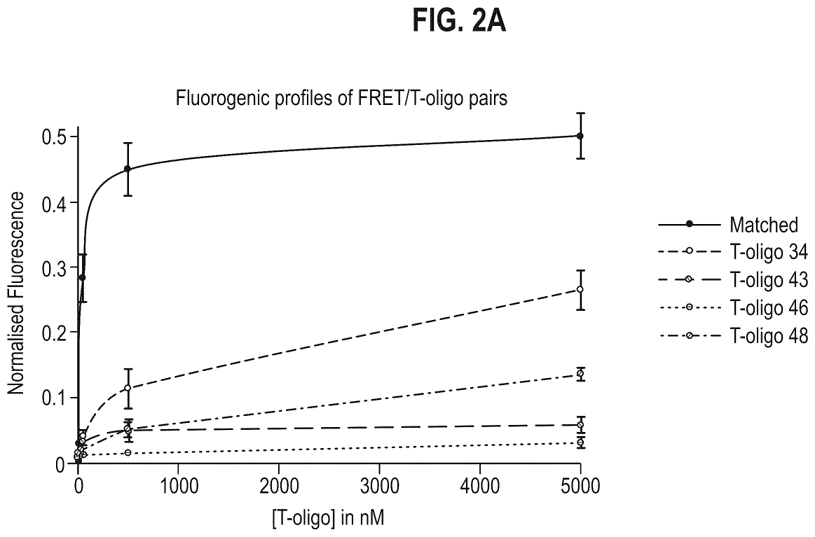

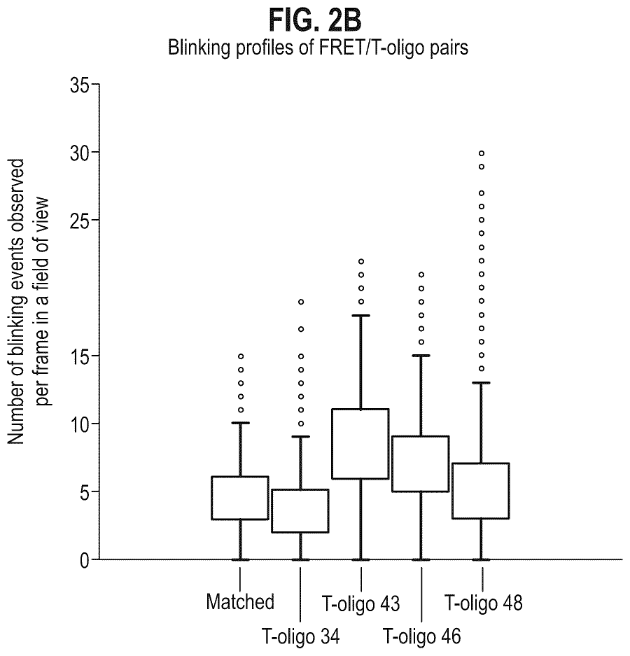

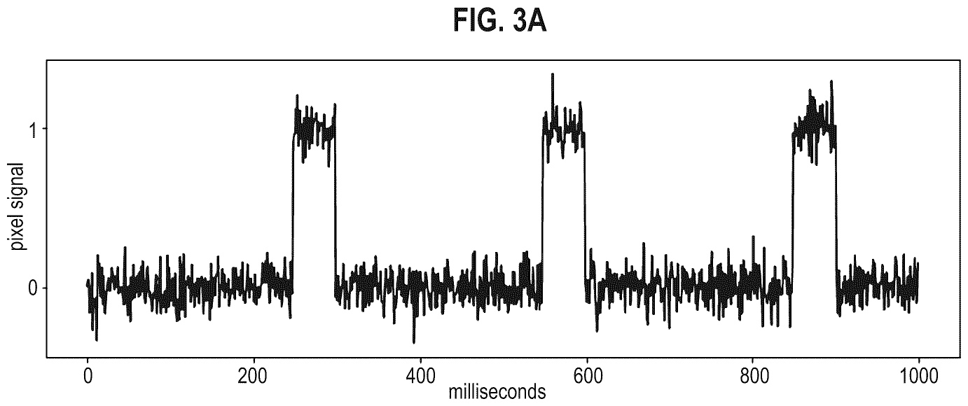

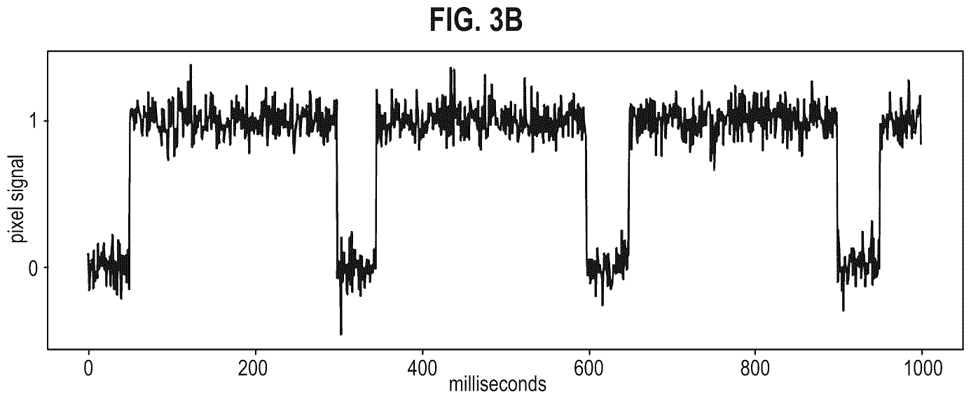

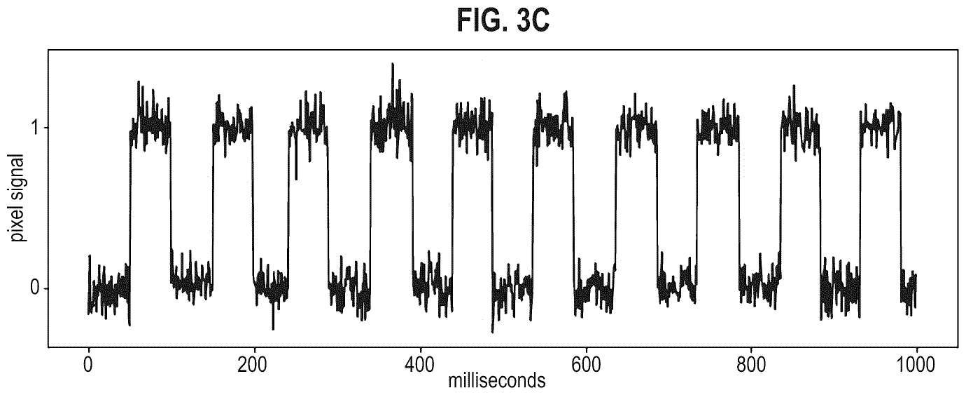

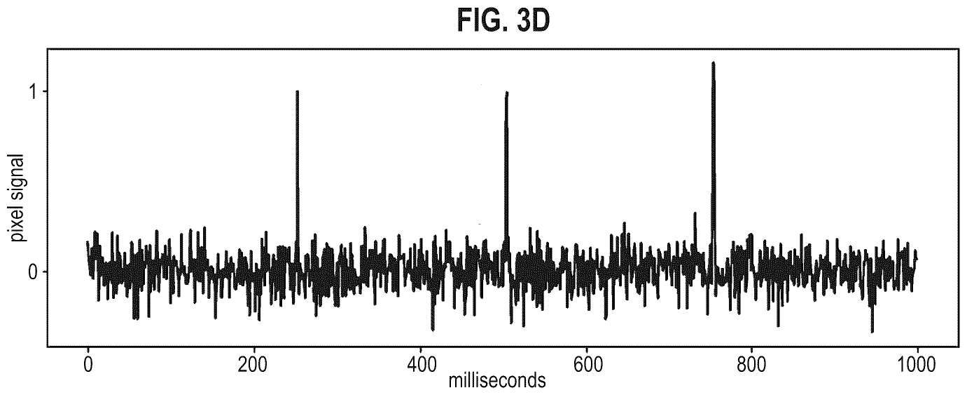

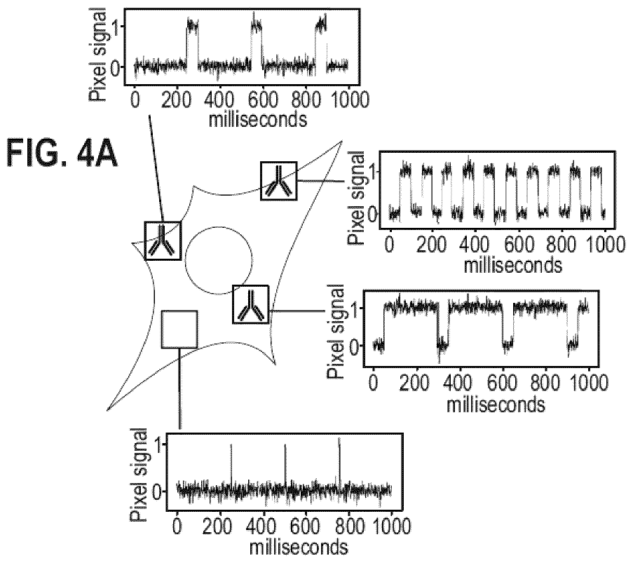

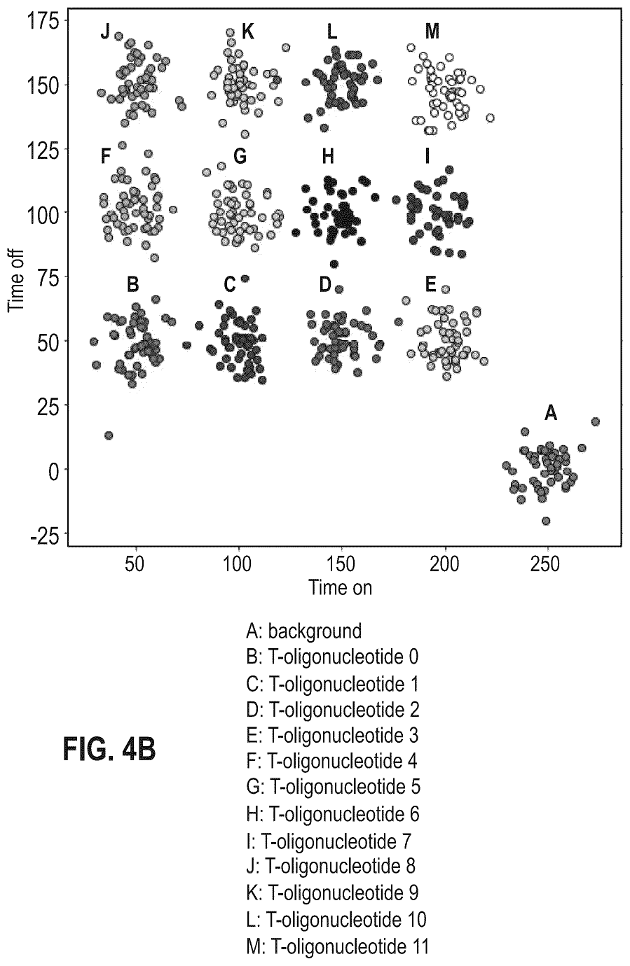

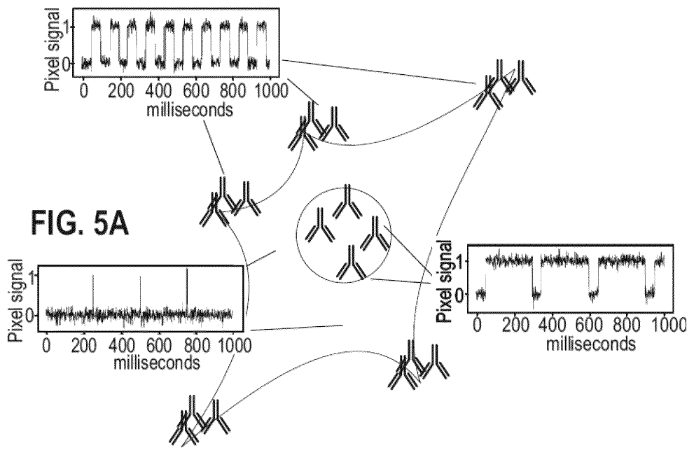

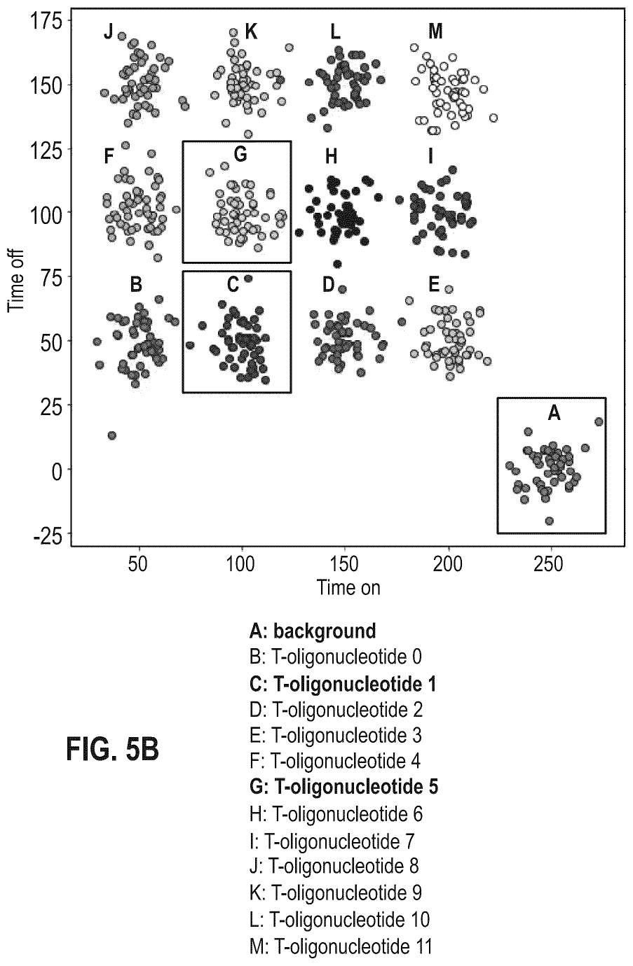

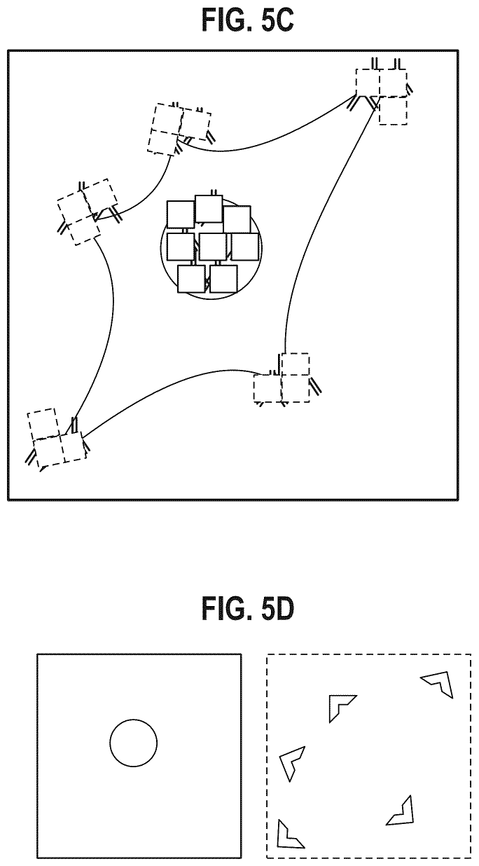

We used this experimental platform and analysis workflow to detect blinking events generated by Cy5-BHQ3/Fret probes bound to the set of T-oligos with different sequence designs and fluorogenic profiles. We observed that the change in the kinetic blinking profile between the different T-oligos corresponded to the inverse fluorogenic profile of the corresponding T-oligo in solution, i.e. T-oligos with fast-blinking kinetics had a low fluorogenic profile in solution, and vice versa (FIG. 2A and FIG. 2B).

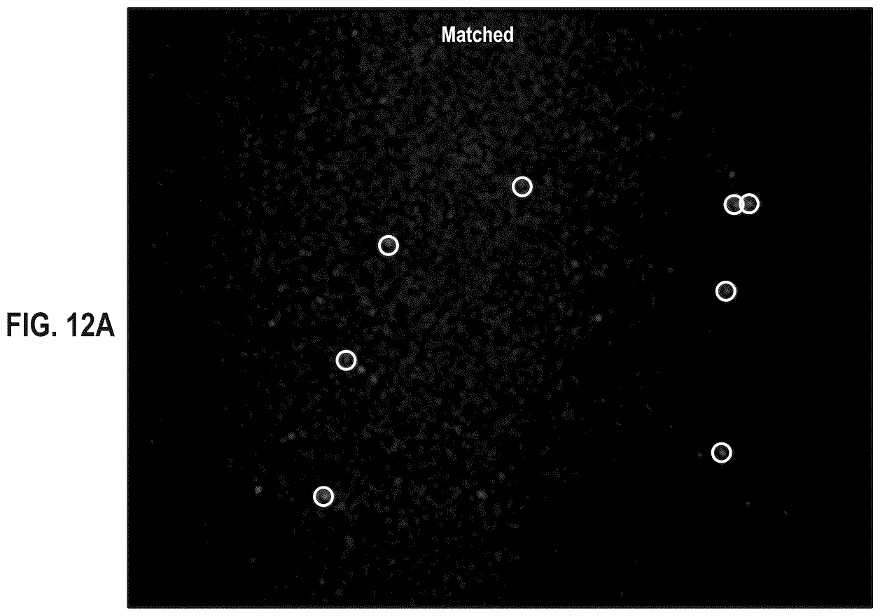

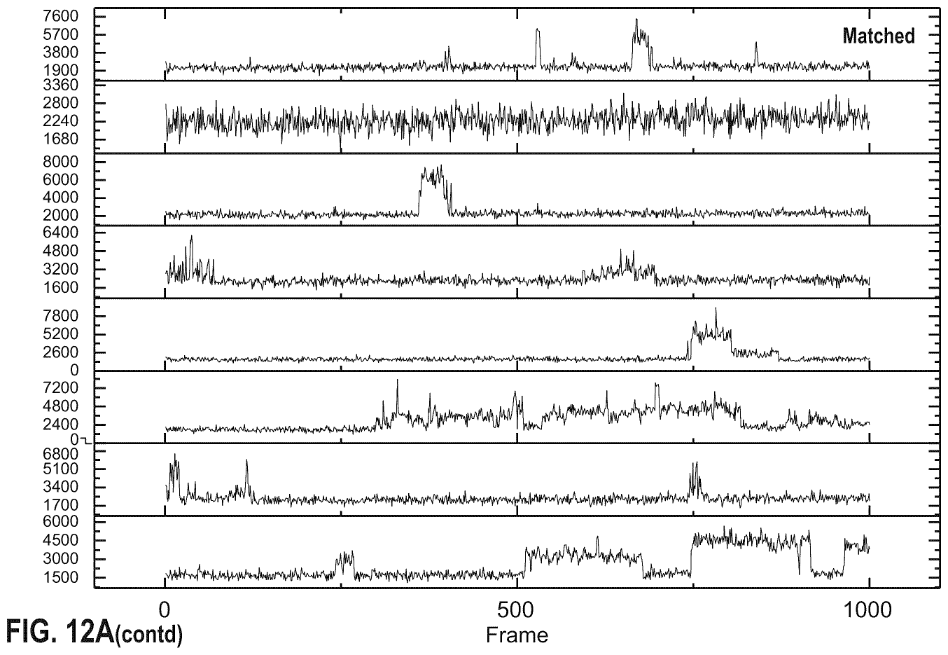

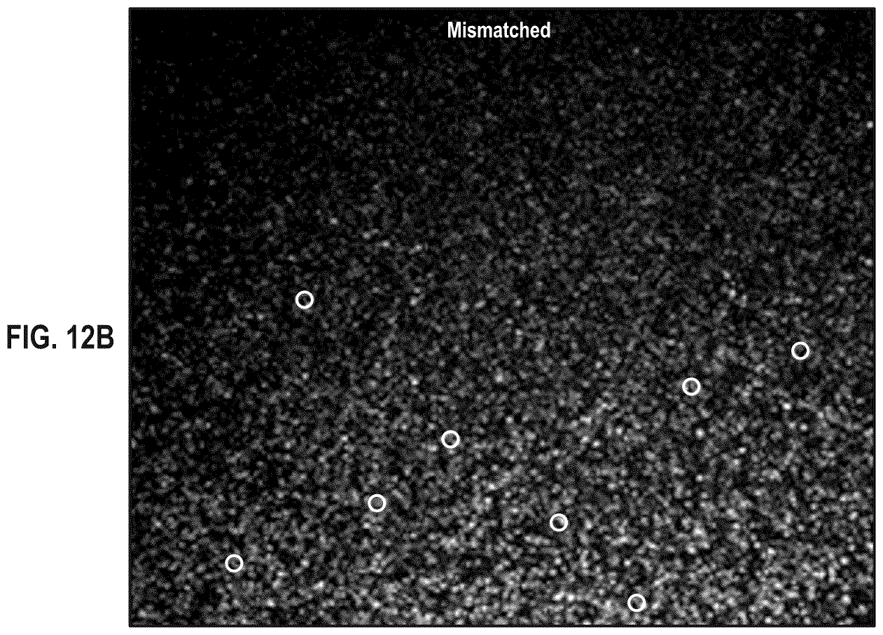

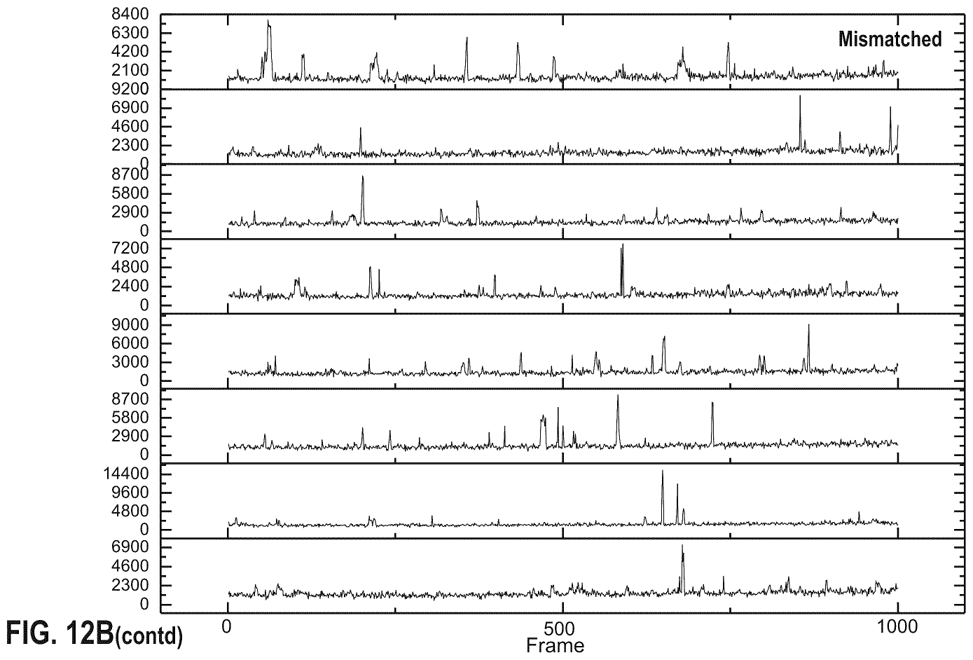

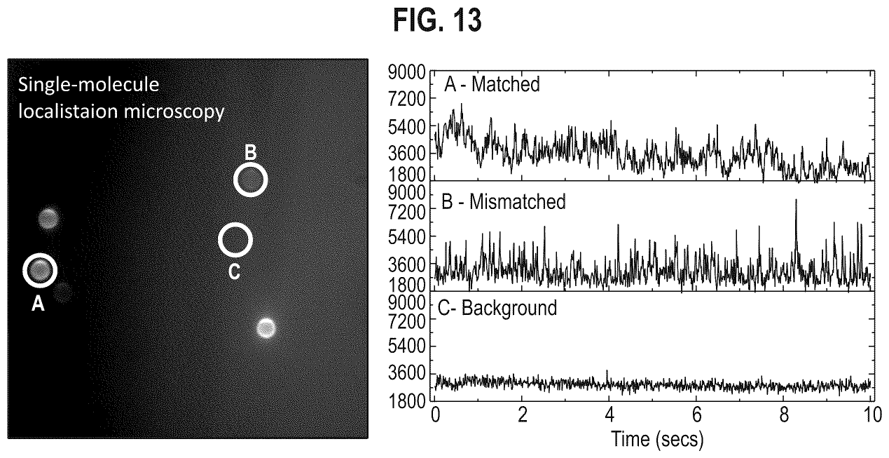

We then analysed the focal kinetic profiles for 8 individual foci for complementary and mismatched T-oligos immobilised on biotin-streptavidin-coated coverslips, allowing multiple blinking events to be captured from individual foci across the acquisition period (1000 frames, 20 ms/frame) without disassociation of the T-oligo from the coverslip surface. Foci were measured with on-time τon ranging from tens of ms up to seconds in blinking complementary T-oligos compared with mismatched T-oligos, whose τon rarely exceeded 100 ms (FIG. 12A and FIG. 12B). The average number of blinking events during the total duration of exposure is also lower for the matched T-oligonucleotide (1.8) compared to the mismatched T-oligonucleotide (4.6). This demonstrates two exemplary metrics that could be used to deconvolute the T-oligonucleotide identity.

Finally, a mixture of streptavidin-coated polystyrene beads conjugated with mismatched and complementary T-oligos were immobilised in a single well on BSA/biotin coated glass and imaged in TIRF mode in the presence of 100 nM FRET-oligo probes. For each measurement, fluorescence from single foci were recorded across a thousand 20 ms exposures. As seen previously with the T-oligos immobilised on glass coverslips, foci were identified with multiple blinking events during the acquisition, representing immobilised T-oligos on the bead surface. Foci with long τon could be identified on beads coated with complementary T-oligos, compared with shorter τon detected on beads coated with mismatched T-oligos (FIG. 9A and FIG. 9B).

### Conclusions

The systematic fluorogenic profiling of 90 FRET-oligo/T-oligo pairs in solution generated by introducing 1-6 mismatches at random positions in the fully complementary T-oligo showed that the number of mismatches correlate with the fluorogenicity of the pair: the more mismatches the T-oligos contain, the less fluorogenic the FRET-oligo becomes upon hybridisation to the T-oligo, presumably by destabilising the binding between the FRET-oligo and the T-oligo. We also found that the position of the mismatches influenced the extent to which the fluorogenic profile of the FRET-oligo was affected. Mismatches in the central part of the T-oligo sequence seemed to have a greater impact on the FRET-oligo's fluorogenicity. Furthermore, adjacent mismatches had a stronger effect, possibly by creating larger bulges and thus further destabilising the hybridisation pairs.

We then assessed whether it was possible to establish a correlation between the fluorogenic profile of a FRET-oligo/T-oligo pair and the rate of blinking, such that the on/off rate of a designated pair could be predicted simply by profiling its fluorescence in solution. We analysed the blinking profiles generated upon hybridisation of the FRET-oligo to immobilised T-oligos. We selected 6 different T-oligos whose pairing to the FRET-oligo lead to distinct fluorogenic profiles. We observed that the T-oligos generating low fluorogenic profiles in solution upon pairing with the FRET-oligo showed high fast-blinking rates when surface-immobilised, while pairs with high fluorogenicity displayed a low blinking rate. This suggested an inverse correlation between fluorogenic profiles and blinking kinetics profiles. This was further confirmed by analysing the focal kinetic profiles of the FRET-oligo when added to a fully complementary T-oligo or to a mismatched T-oligo this time immobilised on beads to circumvent potential nonspecific blinking. Two distinct types of foci were identified: foci with long τon and foci with short τon, corresponding to hybridisation of the FRET-oligo to fully complementary and mismatched immobilised T-oligos, respectively. These results successfully demonstrated that focal kinetic profiles can be established for different FRET-oligo/T-oligo pairs imaged simultaneously.

### Example 3

### Example 3—Spectral Independent Multiplexed Imaging (SIMI) of 6 Cellular Targets on Fixed Cells

T-oligos (1-6, see Table 3) were covalently attached to 6 primary antibodies recognising 6 different epitopes within the cell, forming 6 primary antibody-T-oligo conjugates.

TABLE 3

| | Cellular | | | | | |
|---|---|---|---|---|---|---|
| | Target | | Corresponding T-oligo | | Corresponsing FRET-oligo | |
| Set | Nr | Name | Name | Sequence | Name | Sequence |
| 1 | 1 | α-tubulin | a | TTCCACATTACTTCT | A | 5′ AGAAGTAATGTGGAA |
| | | | | (SEQ ID NO: 1) | | (SEQ ID NO: 7) |
| | 4 | Ki-67 | b | ATCCCCATTACTTCT | | |
| | | | | (SEQ ID NO: 2) | | |
| 2 | 2 | calnexin | C | GGGTGGCGTATGATAGCTAT | B | 5′ ATAACTATCATACTTAACCT |
| | | | | (SEQ ID NO: 3) | | (SEQ ID NO: 8) |
| | 3 | Golgin-97 | d | AGGTTAAGTATGATAGTTAT | | |
| | | | | (SEQ ID NO: 4) | | |
| | 5 | Myosin IIB | e | GGGTTAAGCATGGTAGTTAT | | |
| | | | | (SEQ ID NO: 5) | | |
| | 6 | Tomm-20 | f | AGGTTAAGCGTTATATTTAT | | |
| | | | | (SEQ ID NO: 6) | | |

Commercial human cancer cells were fixed and permeabilised (see Materials and Methods). Primary antibody-T-oligo conjugates were then contacted with the fixed, permeabilized sample. Cells were washed to remove unbound antibody-T-oligo conjugates. Two FRET-oligos (A and B, see Table 3), both conjugated with a Cy5 fluorophore at the 5′end and a BHQ-3 dark quencher at the 3′end, were then incubated with the sample.

The two FRET-oligos can each reversibly hybridize with antibody-T-oligonucleotide conjugates within their set, i.e. T-oligos a-b can hybridize with FRET-oligo A within set 1 and T-oligos c-f can hybridize with FRET-oligo B within set 2.

The sample was illuminated at Cy5's excitation wavelength. For each FRET-oligo, when it is not hybridised with one of its respective T-oligos, the quenching entity BHQ-3 suppresses the fluorescence emission from the emitter fluorophore, Cy5. Upon hybridisation, this quenching is released, allowing detection of fluorescence at the site of hybridisation.

A high-speed CMOS sensor acquires images of the sample over a matter of seconds to minutes in a longitudinal series of images (FIG. 14A). Each of the foci is monitored for fluorescence over time, and a kinetic fluorescence profile is generated. The fluorescence profiles (or one or more metrics thereof) at a specific foci are used to determine that foci's fluorescent kinetic profile. In this example, a foci is 2-3 pixels in diameter. Each pixel's identity is assigned to one of the FRET-/T-oligo pairs, or to background.

The fluorescent kinetic profile at each pixel (example traces are shown in FIG. 14C) can be assigned an identity (e.g. to the specific T-oligonucleotide that has generated it), since each different FRET-oligonucleotide T-oligonucleotide pair generates a fluorescent kinetic profile that is unique to that pair. In this way an identity is assigned to the pixel based on the fluorescent kinetic profile (e.g. as defined with respect to at least one metric thereof) of the sample at that pixel.

Super-resolution images were reconstructed from the cumulated frames using standard localisation algorithms such as those found in ThunderSTORM (Ovesny et al. Bioinformatics 2014)), QC-STORM (Li et al, Opt. Express 2019) and Picasso (Schnitzbauer et al, Nature Protocols 2017) softwares (FIG. 14A). In this example, demultiplexing was performed on the super-resolved images (FIG. 14B); however, demultiplexing is possible with or without super-resolution reconstruction. Target assignment was conducted using standard cluster-based mathematical approaches upon spatial-temporal metrics derived from each FRET-oligo/T-oligo associated blinking profiles. FIG. 14D shows a scatter plot of two extracted features (e.g. blink duration time, and off-time) upon which demultiplexing can be performed (FIG. 14D).

In this example, only one type of fluorophore (Cy5) is used to label FRET-oligos, and therefore the sets of pairs of FRET-oligos and antibody-T-oligonucleotide conjugates shown in Table 3 form one group of sets of pairs (group 1) with the same “colour” (i.e., these are observed via a single channel). In other examples, further groups of sets could be added (e.g. groups 2, 3 . . . ) in which pairs that generate similar or identical profiles to those found in group 1, if the FRET oligos of the additional group emit in a different “colour”, e.g. by using Cy3 as the fluorophore to label the FRET-oligos. This additional level of multiplexing would then be deconvoluted by acquiring images using multiple different imaging channels.

### Materials and Methods

### Oligos

Two FRET-oligos were used, of 15-nucleotide and 20-nucleotide length, respectively, both conjugated with a Cy5 fluorophore at the 5′end and a BHQ-3 dark quencher at the 3′end. T-oligos were generated by introducing mismatches to otherwise fully complementary sequences at random positions using a Python script. The sequences of the T-oligos are given in Table 3. T-oligos and FRET-oligos were purchased from IDT, resuspended in 100 μM TE buffer (1M Tris-HCl, pH 8.0, 0.5M EDTA pH8.0) and stored as aliquots at −20° C.

### Primary Antibody Conjugation

For each target, 15 μg of commercial primary antibodies against 1) α-tubulin, 2) calnexin, 3) Golgin-97, 4) Ki-67, 5) Myosin IIB and 6) Tomm-20 were used for conjugation to a distinct T-oligo, with 800 pmol of oligos supplemented per conjugation. Antibody conjugates were then purified on 50 kDa Amicon Ultra-0.5 columns and then stored at 4° C.

### Immuno-Labeling

Cells were seeded in 18-well glass-bottom μslides (Ibidi) and were fixed with 4% PFA when ˜80% confluent. Cells were incubated in blocking buffer (1×PBS, 1% BSA, 0.1% Triton-X, 0.2 mg/ml sheared salmon sperm DNA) for 60 min at room temperature and then incubated overnight at 4° C. with T-oligo-antibody mix, each T-oligo-antibody conjugate diluted 1/100-1/200 in 1×PBS, 1% BSA, 0.1% Triton-X, 0.1 mg/ml sheared salmon sperm DNA, 150 mM NaCl, 5 mM EDTA, 0.05% dextran sulfate.

### Imaging

Immuno-labeled cells were incubated with 5 nM of FRET-oligo diluted in imaging buffer (1×PBS, 10 mM Tris-HCl pH 8, 500 mM NaCl, 1 mg/ml glucose oxidase, 5 mg/ml glucose, 0.04 mg/ml catalase, 1 mM Trolox). 10 000 images at 10 ms/frame, 16 bit, 2-CMS gain mode were acquired on a Nikon 60×1.49NA TIRF objective, filtered through a Cy5 filter set, equipped with a Prime BSI Express sCMOS Camera. Final super-resolution images were reconstructed using QC-STORM or Picasso softwares. Demultiplexing to obtain identity of individual targets from each channel was then performed using standard cluster-based mathematical approaches upon spatial-temporal metrics derived from each FRET-oligo/T-oligo associated blinking profiles.

1. A method for multiplexed fluorescence microscopy comprising:
a. contacting a fixed sample with a set of binding agent-T-oligonucleotide conjugates to allow the binding agents to bind to any binding partners present in the sample, wherein the set comprises a plurality of binding agents having different specificities and the sequence of the T-oligonucleotide is unique to the binding agent to which it is conjugated;
b. contacting the sample and any bound binding agents resulting from step a with a FRET-oligonucleotide;
c. illuminating the sample with a wavelength to cause excitation of the FRET-oligonucleotide's emitter molecule; and
d. observing the fluorescent kinetic profile of the sample at the FRET-oligonucleotide emitter molecule's emission wavelength at one or more pixels over time;
wherein the FRET-oligonucleotide can hybridise to multiple T-oligonucleotides in the set, to form multiple pairs, and wherein the dissociation and reassociation between each different pair generates a fluorescent kinetic profile that is unique within that set to that pair.

2. The method of claim 1 further comprising the calculation of a distinguishable metric from the fluorescent kinetic profile and optionally assigning an identity to one or more pixels in the observed sample based on the metric calculated from the fluorescent kinetic profile of the sample at that pixel.

3. The method of claim 1, wherein unbound binding agent-T-oligonucleotide conjugates are removed after step a; or
wherein the sample is re-fixed after step a to further immobilise any bound binding agents.

4. (canceled)

5. The method of claim 1, wherein:
a. each T-oligonucleotide in the set is 8 to 35 nucleotide units in length; and/or
b. the FRET oligonucleotide is (i) of a length that exceeds the Forster Radius of the FRET-oligonucleotide emitter molecule, and/or is (ii) at least 12 nucleotide units in length; or
wherein each T-oligonucleotide can hybridise to the FRET oligonucleotide and each T-oligonucleotide has 0 to 40% of base pair mismatches with the FRET oligonucleotide.

6. (canceled)

7. The method of claim 1 wherein each of the binding agents is a proteinaceous molecule, optionally an antibody or fragment thereof.

8. The method of claim 2, wherein at least two metrics are calculated from the fluorescent kinetic profile and used to assign an identity to one or more pixels in the observed sample, or
wherein at least one metric calculated or derived from the fluorescent kinetic profile of each different FRET-oligonucleotide and T-oligonucleotide pair is selected from:
(i) the average period of time between each fluorescence emission;
(ii) the average duration of the fluorescence emission;
(iii) the rate of occurrences of fluorescence emission;
(iv) the dissociation constant (Koff) of the FRET-oligonucleotide to T-oligonucleotide binding as inferred from the fluorescent kinetic profile; and
(v) the association constant (Kon) of the FRET-oligonucleotide to T-oligonucleotide binding as inferred from the fluorescent kinetic profile and component concentrations.

9. (canceled)

10. The method of claim 1, wherein the fluorescent kinetic profile is observed using a high-frame rate imaging device capable of detecting single molecule blinking events.

11. The method of claim 1, wherein:
(i) step a comprises contacting the sample with a group of sets of binding agent-T-oligonucleotide conjugates, said group of sets comprising a first set of binding agent-T-oligonucleotide conjugates and one or more subsequent sets of binding agent-T-oligonucleotide conjugates; and
(ii) step b comprises contacting the sample and any bound binding agents resulting from step a with a first FRET-oligonucleotide and one or more subsequent FRET-oligonucleotides,
wherein said first FRET-oligonucleotide can hybridise to multiple T-oligonucleotides in the first set, to form multiple pairs, and wherein said each subsequent FRET-oligonucleotide can hybridise to multiple T-oligonucleotides in its corresponding set, to form multiple pairs, and wherein the dissociation and reassociation between each different pair generates a fluorescent kinetic profile that is (i) unique within each set to that pair, and (ii) unique within the group of sets to that pair.

12. The method of claim 11, wherein:
the first and the subsequent FRET oligonucleotides contain emitter molecules which have overlapping emission spectrums, optionally which have the same emitter molecule; or wherein:
(i) step a comprises contacting the sample with multiple sets of binding agent-T-oligonucleotide conjugates; and wherein the emission spectrum of the emitter molecules in the corresponding FRET oligonucleotides for each set of binding agent-T-oligonucleotide can be detected in a channel that is specific to that set; or
(ii) step a comprises contacting the sample with
(I) one or multiple sets of binding agent-T-oligonucleotide conjugates, wherein the emission spectrum of the emitter molecules in the corresponding FRET oligonucleotide for each set of binding agent-T-oligonucleotide can be detected in a channel that is specific to that set and
(II) one or multiple groups of sets of binding agent-T-oligonucleotide conjugates wherein the emission spectrum of the emitter molecules in the corresponding FRET oligonucleotides for each group of sets of binding agent-T-oligonucleotide can be detected in a channel that is specific to that group of sets; and
the step of observing the fluorescent kinetic profile of the sample comprises observing the fluorescent kinetic profile of the sample in a channel for each set or for each group of sets.

13. The method of claim 11 or 12, wherein step a comprises contacting the sample with multiple groups of sets of binding agent-T-oligonucleotide conjugates, each group of sets comprising a first set of binding agent-T-oligonucleotide conjugates and one or more subsequent sets of binding agent-T-oligonucleotide conjugates; and wherein the emission spectrum of the emitter molecules in the corresponding FRET oligonucleotides for each group of sets of binding agent-T-oligonucleotide can be detected in a channel that is specific to that group of sets and the step of observing the fluorescent kinetic profile of the sample comprises observing the fluorescent kinetic profile of the sample in a channel for each group of sets.

14. The method of claim 13 wherein, for each group of sets of binding agent-T-oligonucleotide conjugates, the corresponding first and subsequent FRET oligonucleotides contain the same emitter molecule.

15. (canceled)

16. The method of claim 12 wherein:
(i) step a comprises contacting the sample with multiple sets of binding agent-T-oligonucleotide conjugates wherein for each set of binding agent-T-oligonucleotide conjugates the corresponding FRET oligonucleotide contains an emitter molecule that is specific to each set; or
(ii) step a comprises contacting the sample with
(I) one or multiple sets of binding agent-T-oligonucleotide conjugates, wherein for each set of binding agent-T-oligonucleotide conjugates the corresponding FRET oligonucleotide contains an emitter molecule that is specific to each set; and
(II) one or multiple groups of sets of binding agent-T-oligonucleotide conjugates wherein, for each group of sets of binding agent-T-oligonucleotide conjugates the corresponding first and subsequent FRET oligonucleotides contain the same emitter molecule; and
the step of observing the fluorescent kinetic profile of the sample comprises observing the fluorescent kinetic profile of the sample in a channel for each set or for each group of sets.

17. The method of claim 11, wherein each set of binding agent-T-oligonucleotide conjugates comprises 2 to 25 different binding agent-T-oligonucleotide conjugates; or
wherein each group of sets of binding agent-T-oligonucleotide conjugates comprises 2 to 25 sets of binding agent-T-oligonucleotide conjugates.

18. (canceled)

19. The method of claim 1, wherein at least one set of binding agent-T-oligonucleotide conjugates comprises:
a. one or both of the T-oligonucleotides of sequence SEQ ID NO:1 and SEQ ID NO:2, and the corresponding FRET-oligonucleotide is a FRET-oligonucleotide of sequence SEQ ID NO:7; and/or
b. at least one set of binding agent-T-oligonucleotide conjugates comprises at least one of the T-oligonucleotides of sequence SEQ ID NO:3, SEQ ID NO:4, SEQ ID NO:5 and SEQ ID NO:6 and the corresponding FRET-oligonucleotide is a FRET-oligonucleotide of sequence SEQ ID NO:8.

20. A kit for multiplexed fluorescence microscopy comprising:
a. a set of binding agent-T-oligonucleotide conjugates, wherein the set comprises a plurality of binding agents having different specificities and the sequence of the T-oligonucleotide is unique to the binding agent to which it is conjugated; and
b. a FRET-oligonucleotide
wherein the FRET-oligonucleotide can hybridise to multiple T-oligonucleotides in the set to form multiple pairs, and wherein the dissociation and reassociation between each different pair generates a fluorescent kinetic profile that is unique within that set to that pair.

21. The kit of claim 20, further comprising one or more additional sets of binding agent-T-oligonucleotide conjugates, and one or more additional FRET oligonucleotide, wherein said each further FRET-oligonucleotide can hybridise to multiple T-oligonucleotides in its corresponding set, to form multiple pairs and wherein the dissociation and reassociation between each different pair generates a fluorescent kinetic profile that is unique within each set to that pair; or
wherein at least one set of binding agent-T-oligonucleotide conjugates comprises:
one or both of the T-oligonucleotides of sequence SEQ ID NO:1 and SEQ ID NO:2, and the corresponding FRET-oligonucleotide is a FRET-oligonucleotide of sequence SEQ ID NO:7; and/or
at least one set of binding agent-T-oligonucleotide conjugates comprises at least one of the T-oligonucleotides of sequence SEQ ID NO:3, SEQ ID NO:4, SEQ ID NO:5 and SEQ ID NO:6 and the corresponding FRET oligonucleotide is a FRET-oligonucleotide of sequence SEQ ID NO:8.

22. (canceled)

23. A set of binding agent-T-oligonucleotide conjugates and a corresponding FRET oligonucleotide for making a kit for multiplexed fluorescence microscopy comprising:
a. a set of T-oligonucleotides; and
b. a FRET-oligonucleotide
wherein the FRET-oligonucleotide can hybridise to multiple T-oligonucleotide in the set to form multiple pairs, and wherein the dissociation and reassociation between each different FRET-oligonucleotide and T-oligonucleotide generates a fluorescent kinetic profile that is unique within that set to that pair.

24. The set of binding agent-T-oligonucleotide conjugates and a corresponding FRET-oligonucleotide for making a kit for multiplexed fluorescence microscopy of claim 23, wherein:
the set of binding agent-T-oligonucleotide conjugates comprises
a. one or both of the T-oligonucleotides of sequence SEQ ID NO:1 and SEQ ID NO:2, and wherein the corresponding FRET-oligonucleotide is a FRET-oligonucleotide of sequence SEQ ID NO:7; or
b. The set of binding agent-T-oligonucleotide conjugates comprises at least one of the T-oligonucleotides of sequence SEQ ID NO:3, SEQ ID NO:4, SEQ ID NO:5 and SEQ ID NO:6, and wherein the FRET-oligonucleotide is a FRET-oligonucleotide of sequence SEQ ID NO:8.

25. A method of preparing the kit according to claim 20, said method comprising providing a set of T-oligonucleotides and a set of binding agents and a corresponding FRET oligonucleotide, and conjugating the set of T-oligonucleotides to the set of binding agents to form a set of binding agent-T-oligonucleotide conjugates, wherein the set of binding agent-T-oligonucleotide conjugates comprises a plurality of binding agents having different specificities and the sequence of the T-oligonucleotide is unique within that set to the binding agent to which it is conjugated.

26. A method of designing a set of T-oligonucleotides and a FRET-oligonucleotide for use in the method of claim 1, said method comprising:
a. selecting a FRET-oligonucleotide sequence of at least 12 nucleotides in length;
b. obtaining one or more sequences of at least 8 nucleotides in length that are complementary to the FRET-oligonucleotide sequence;
c. generating a plurality of potential T-oligonucleotide sequences each of which differs from the sequence that is complementary to the FRET-oligonucleotide by at least one nucleotide; and
d. selecting at least two T-oligonucleotide sequences on the basis of the ability of the at least two T-oligonucleotide sequences to generate fluorescent kinetic profiles that are different to each other.
